SEQUENCE LISTING (60 sequences)

SEQ ID NO: 1 - protein, 520 aa, Penicillium emersonii
MLRRALLLSSSAILAVKAQQAGTATAENHPPLTWQECTAPGSCTTQNGAVVLDANWRWVHDVNGYTNCYTGNTWNPTYCPDDETCAQNCALDGADYEGTYGVTSSGSSLKLNFVTGSNVGSRLYLLQDDSTYQIFKLLNREFTFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYVQNGNVIPQPNSDISGVTGNSITTEFCTAQKQAFGDTDDFSQHGGLAKMGAAMQQGMVLVMSLWDDYAAQMLWLDSDYPTDADPTTPGIARGTCPTDSGVPSDVESQSPNSYVTYSNIKFGPINSTFTASSNPGGGTTTTTTTQPTTTTTTAGNPGGTGVAQHYGQCGGIGWTGPTTCASPYTCQKLNDYYSQCL

SEQ ID NO: 2 - protein, 503 aa, Aspergillus nidulans
QKVGTQQAEVHPGLTWQTCTSSGSCTTVNGEVTIDANWRWLHTVNGYTNCYTGNEWDTSICTSNEVCAEQCAVDGANYASTYGITTSGSSLRLNFVTQSQQKNIGSRVYLMDDEDTYTMFYLLNKEFTFDVDVSELPCGLNGAVYFVSMDADGGKSRYATNEAGAKYGTGYCDSQCPRDLKFINGVANVEGWESSDTNPNGGVGNHGSCCAEMDIWEANSISTAFTPHPCDTPGQTLCTGDSCGGTYSNDRYGGTCDPDGCDFNSYRQGNKTFYGPGLTVDTNSPVTVVTQFLTDDNTDTGTLSEIKRFYVQNGVVIPNSESTYPANPGNSITTEFCESQKELFGDVDVFSAHGGMAGMGAALEQGMVLVLSLWDDNYSNMLWLDSNYPTDADPTQPGIARGTCPTDSGVPSEVEAQYPNAYVVYSNIKFGPIGSTFGNGGGSGPTTTVTTSTATSTTSSATSTATGQAQHWEQCGGNGWTGPTVCASPWACTVVNSWYSQCL

SEQ ID NO: 3 - protein, 533 aa, Penicillium swiecickii
MASTLSFNIYKNALLFAAFLGAAKAQQVGTNTAEVHPSLTWQKCTAGGTCTSQSGKVVVDANWRWLHSTSGSTNCYTGNTWDATLCPDDVTCATNCALDGADYTGTYGVTASGSSLRLNFVTTASQKNIGSRLYLMENDTKYQMFQLLNQEFTFDVDVSNLPCGLNGALYFVSMDSDGGMARYPANKAGAKYGTGYCDSQCPRDLKFINGQANVEGWVPSSNDVNAGTGNHGSCCAEMDIWEANSISNAVTPHPCDTPTQVQCTGDACGGTYSSDRYGGTCDPDGCDFNPYRQGNQSFYGPSKIVDTTSPFTVVTQFITNDGTSTGTLSEIKRFYVQNGKVIPQSVSTISAVSGNSITDSYCAAQKKAFGDTDVFTKHGGMAGMGAGLDEGMVLVMSLWDDHAANMLWLDSIYPTTATSTTPGAARGSCDISSGTPTDVEANHANAYVIYSNIKVGPLGSTFNSGSSGSGTTTTTTTKATTTTTTKATTTTTGSTTTGAAHYAQCGGSSWTGATTCASPYTCQKQNDFYSQCL

SEQ ID NO: 4 - protein, 456 aa, Talaromyces verruculosus
MLRYLSIVAATAILTGVEAQQSVWGQCGGQGWTGATSCAAGSTCSTLNPYYAQCIPATATSTTLATTTSSTSVGTTSPPTTTTTKATTTATTAAASGNPFSGYQLYANPYYSSEVHTLAIPSLTGTLAAAATKAAEIPSFVWLDTAAKVPTMGTYLANIQAANKAGASPPIAGIFVVYDLPDRDCAAAASNGEYTVANNGVANYKAYIDSIVAQLKAYPDVHTILIIEPDSLANMVTNLSTAKCAEAQSAYYECVNYALINLNLANVAMYIDAGHAGWLGWPANLTPAAQLFAAVYKNASAPAALRGLATNVANYNAWSISSPPSYTSGDSNYDEQLYINALSPLLTANGWPNAHFIMDTSRNGVQPTKQQAWGDWCNVIGTGFGVPFTTSTGDALEDAFVWVKPGGESDGTSNSSSTRYDYHCGYSDALQPAPEAGTWFQAYFAQLLTNANPALA

SEQ ID NO: 5 - protein, 410 aa, Cladosporium antareticum
MKSSFIAVAAASTVGSAYAQGQAYAQCGGVGWSGSTSCVSGYTCTKTNDYYSQCLPGGGSNPTTTTRPSTTTTSRTGGSPTGGSGSGRVSNAGVNIAGLDFGCTIDGTCKTANIVDPGQIGIDQMQHFVKDDGLNTFRIPVGWQFLLNNNLGGTLDSNNWNTYDKQIQGCLNTGAALCIVDIHNYARWNGAIVGQGGPTNAQFTSLWSQIAAKYKGNNKIAFGLMNEPHDLNINTWAQTVQEAVTAIRKAGATSNKILLPGTDFTSAGAFISNGSGGALIKVTNLDGSTTNLIFDVHKYLDSDNSGTHTTCTTNNAEAFTSLGNWLRTNKRQAILTETGGGPNDASCLKAVCEQLDVLNSYSDVYLGWTGWSAGRFDATYELTETPTRNGNGGWTDVPLVTQCIAGKFKR

SEQ ID NO: 6 - protein, 855 aa, Talaromyces pinophilus
MRNSLLISLAAAALAEGKAYSPPAYPAPWASGAGEWAQAHQRAVEFVSQLTLAEKINLTTGVGWEGGQCVGNTGSIPRLGFRSLCMQDSPLGVRDTDYNTAFPAGVNVAATWDLDLAYRRGIAMAEEHRGKGVDVQLGPVAGPLGRVPEGGRNWEGFAPDPVLTGQMMASTIQGMQDTGVIACAKHYIGNEQEHFRQGSQENFTVADAISSNIDDITLHELYLWPFADAVRAGVGSVMCSYNQINNSYSCGNSYTLNHILKGELDFQGFVMTDWSAQHSGVGDALAGADMDMPGDVAFDSGTAFWGTNLTIAVLNGTVPEWRVDDMAVRIMSAFYKVGRDRTQVPINFASWTLDTYGNEYYYAGEGYKEINQHVDVRGGHAKVVREIGSASIVLLKNVDGALPLTGSEKFVAVFGEDAGSNPDGVNGCSDRGCDNGTLAMGWGSGTANFPYLVTPEQAIQAEVVKNGGMFTAITDSGATDTAKTVAAQASACLVFANADSGEGYITVDGNVGDRKNLTLWQNGEAMISAVAGNCNNTIVVLHTVGPVLVEDWVHHPNITAVLWAGLPGEQSGNSLVDVLYGSVNPGGKTPFTWGKQRSDWGVDVIYEPNNGDGAPQQDFTEGIFIDYRHFDKYNITPTYEFGYGLSYSTFSFSDLKVTPIAASPYRPAKGQSGPAPVLGKVLNATAYLFPDYIKRIEAFIYPWLNSTDLKTSSGDPNYGWPTSKYVPDGAQDGSPQPVNPAGGAPGGNPALYDPVAEVTVTVKNTGKVAGVEVPQLYVSLGGPSDAPKVLRGFGRISLGAGEETQWTATLTRRDVSNWDTVRQNWVVTNYTKTVYVGNSSRNLPLQQTLALNIGH

SEQ ID NO: 7 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 8 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 9 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 10 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 11 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 12 - protein, 1020 aa, Talaromyces funiculosus
LPFNERVDQVLRSYEVTSKLDSRSTKPSKHGHTYQTQFLGVTWDQRNWRLQSTVLDQGHYESRGSIANGYIGLNVAGAGPLFELDSPVDGDVINGWPLFSRRQTFAGLAGFYDLQPRTNGTNFPWLSQYGDDSAISGVPHWGGMVLDLGDGEYLDATVDNSTISDYTTTYDYKAGVLSWDYKWTPKNANGSFGISYKIFANKLDVNQAVVQLSITPSTNGSASVVNVIDGYAAVRTDFVSSGNESDVVYTAVKPNGVTNVTAWIYTALDGDDAFDISSAALVNDKPYVHQNDSSIAQSVNVTFTAGTTITINKFVGAASTDAFPDPQSTAREAALSARRRGFDDLFRSHISEWAQVMPDDSVDDFTLANGTLPNDTFIIESAVMAVVNPYYLLQNTVGPNALRRVNNAPVNDWSIPVGGLTSDSYAGQIFWDADVWMQPGLVAAFPESAKRITNYRAAKYSQALENAKTAYTSSQNQTWFSPDAAIYSWTSGRVGNCTATGPCWDYEYHLNGDIGISLVNEWVVSGDNETFKNKHFPIYNSIATLYGDLLKKNGSYYTLTNMTDPDEYANNVDAGGYTMTLISQTLSNANAFRKQFGMNENTTWTEMADNILLIRENDVTLEYTTMNNSVAVKQADVILSTFPLDYTKNYTTSAALNDLDYYALKQSPDGPGMTYAIFSIVANDVSPSGCSAYTYAQYSYDPYIRGPFFQFSEQLLDDYTINGGTHPAFPFLTGHGGANQVVLYGYLGLRLLPDDMLHIDPNLPPQIPSIKYRTFYWRGWPIQAASNYTHTTIQRATTVAPLSTADPTYANKSIHVSVGHNTVNSTTYSLSANGSALVVPNRQIGSINTVAGNVVQCKSVLSTDAYQKGQYPISAVDGAASTKWQPEFAANISSLTVDLTGSNVSSVSGFYFDWAQAPPTNITVLLHNSSSAALASSGDKPGSSAVTLNITISNPYNASTYNANIIALPSSNSTNYTFPAPVPKPRYATLFVQGNQALDETDTKSGNGTGATVAEWAILS

SEQ ID NO: 13 - protein, 556 aa, Gloeophyllum sepiarium
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 14 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 15 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 16 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSS

SEQ ID NO: 17 - protein, 325 aa, Palaeococcus ferrophilus
GKPKPSQPPQEVPWGIERVKAPSVWSTTDGSSNGVIQVAILDTGIDYDHPDLAANLAWGVSTLRGRVSTKPKDYRDQNGHGTHVAGTIAALNNDIGVVGVAPGVQIYAIRVLDASGRGSYSDIAIGIEQAILGPDGVADKDGDGIIAGDPDDDAAEVISMSLGGSADDSYLHDMIIQAYNAGIVIVAASGNEGASSPSYPAAYPEVIAVGASDINDNIASFSNRQPEVSAPGVDVLSTYPDDTYKTLSGTSMATPHVSGVVALIQAAHFNKYGTILPVGTFDDMSKNTVRGILHITADDLGSPGWDVDYGYGIVRADLAVQAALG

SEQ ID NO: 18 - protein, 186 aa, Thermobifida cellulosytica
ADVIGGNPYYFGGYRCSIGFSVRKGSDTGFATAGHCGETGTLTRSPEGVVAGSYFPGRDMGWVRLTGADTVTPLVNRYDGGTVTVTGSQEAVTGSSVCRSGSTTGWRCGIIQSKNQTVRYAEGTVTGLTRTTACAEAGDSGGPWLTGSQAQGVTSGGSGNCRTGGITYFQPINPLLSYFGLELVTG

SEQ ID NO: 19 - protein, 186 aa, Thermobifida fusca
AAIIGGNPYYFGNYRCSIGFSVRQGSQTGFATAGHCGSTGTRVSSPSGTVAGSYFPGRDMGWVRITSADTVTPLVNRYNGGTVTVTGSQEAATGSSVCRSGATTGWRCGTIQSKNQTVRYAEGTVTGLTRTTACAEGGDSGGPWLTGSQAQGVTSGGTGDCRSGGITFFQPINPLLSYFGLQLVTG

SEQ ID NO: 20 - protein, 186 aa, Thermobifida halotolerans
TDIIGGNPYYFDGYRCSIGFSVRRGSESGFATAGHCGEEGTETSDPEGTVAGAYFPGRDMGWVRITDADTVTPLVNRYNGENVTVAGSREAATGSSVCRSGSTTGWRCGTIRSKNQTVRYIEGTVTGLTRTTACAEGGDSGGPWLTGSQGQGVTSGGSGNCTLGGVTYFQPLNPLLSHFDLDLVTG

SEQ ID NO: 21 - protein, 325 aa, Thermococcus nautili
LVPEPTQPAQEIPWGIERVKAPDTWSITTGASNGVIEVAILDTGIDWDHPDLKDNIAWGVSTIGGEVSTDPADWYDGNGHGTHVAGTIAALNNDIGVVGVAPNVEIYAIKVLDDRGSGAYTDIAIGIEQALLGPDGILDKDGDGIIVGDPDDDAAEVISMSLGGPSDDEYLHDMIIKAYNYGVVIVAASGNEAADQPSYPAIYPEVIAVGATDSSDAVAYFSNLQPEVSAPGVDVLSTYPDDTYESLSGTSMATPHVSGVVALIQAAYYNKYGKVLPVGTFDDMNTNTVRGILHVTADDLGDPGWDIYYGYGIVRADLAVQAALG

SEQ ID NO: 22 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 23 - DNA, 1729 bp, Talaromyces verruculosus
atgctgcgatatctttccattgttgccgctacggcaatcttgaccggagtggaagctcagcaatcggtctggggtcaatgtaagaaatctttcgagaagcttccagtccaaattaaattacagctattgacatatttcaggtggtggtcaaggctggactggcgcaacgtcatgcgccgccggttctacatgcagcactctaaacccttgtaagatgccagctgattccattgcacgttggctctgtttactgacagcaattggttcgcagactacgcacaatgtattcctgccacagctacttcaaccacattggcgacaaccacctcttccacaagtgttgggacgacatcgccgccgacaaccaccacgacgaaagctaccaccacagctaccactgccgctgcatctggaaaccccttctctggttatcagctttatgccaatccgtactactcttcagaagtacatactcttgccataccatctctgactggcacactcgccgctgctgcgaccaaggctgcagagattccgtcatttgtctggctgtgagtgtcctcaataatatacaattgagtgttataaatatatgtgtgaacattttctaaacctttatagtgacacagcagctaaagtgcctacaatgggtacctacttggctaacattcaagctgcaaacaaggctggagctagcccacctattgccggtatcttcgttgtctatgacttgcctgaccgtgactgtgcagctgcagcaagtaatggcgaatacacagtagcaaacaacggtgtcgcaaactataaggcttacatcgatagcatcgtggcacagttgaaagcttatcccgatgtgcacaccatcctcatcattggtacgttctccagtactggaccttgaaaaagtacttttcagagaaattcatgtctaacacatcaccgttctacagaacccgacagtctagccaatatggttaccaatttgtctacagccaaatgtgccgaagctcaatctgcatactacgagtgcgtcaactacgcattgatcaacctcaacttggccaacgtggccatgtacattgatgccggtcatgctggttggcttggatggcctgccaatctcacaccagctgctcaactttttgcagcagtctataagaatgcaagtgctcctgcagcacttcgtggactggccaccaacgttgccaactacaacgcttggtcgatcagcagcccaccctcctacacgtctggtgactccaactacgacgaacagctctacatcaatgctttgtctcctctcctaacagcaaacggctggcctaatgctcactttattatggatacttgtaagtgtgccgcaaatgactcatccaagctttcaacttactaatccgaatgtttgttttagcccgaaacggtgttcagccaaccaagcagcaggcatggggagactggtgcaatgtgatcggaaccggcttcggtgttcctttcacaaccagcaccggtgacgcactagaggatgcctttgtctgggtcaaaccaggtggtgaaagtgatggtacctcaaacagttcctctactcgttacgattatcattgcggctacagtgatgcacttcaacctgctcccgaggctggcacttggttccaagcttattttgcccagcttttgaccaatgccaatccagctttggcctagatcagctttgactgcaccaatatccttt

SEQ ID NO: 24 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 25 - protein, 555 aa, Pycnoporus sanguineus
QSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 26 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 27 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 28 - protein, 694 aa, Myceliophthora sepedonium
MALRHIAAAAIAGLASRTAALYINGSVTAPCDSPIYCQGELLKAVELARPFVDSKTFVDMPTIKPVDEVLAAFSKLSLPLSNNSELNAFLYENFAQAGHELEEVPDSELETDAKFLDKLEDRTIKEFVGKVIDIWPDLTRRYAGPSNCTECANSFIPVNRTFVVAGGRFREPYYWDSYWIVEGLLRTGGAFTHISKNIIENFLDFVDTIGFIPNGARIYYLNRSQPPLLTLMVKSYVDYTNDTSILDRALPLLIKEHEFFMNNRTVSITGSNGKEYTLNRYHVENNQPRPESFREDYITANNGSYYASSGIIYPVKTPLNETEKAALYSNLATGAESGWDYTSRWLGVPSDAARDVYFPLRSLNVRDIVPVDLNSILYQNEVIIAEYLEKAGNSSAAKRFATAAEQRSEAMYSLMWNATHWSYFDYNLTDNTQHIFVPADEDTAPQDRIEAPPGQQVFFHIAQLYPFWTGAAPASLKANPLAVQQAYARVARMLDIKKGAIPATNYRTGQQWDQPNVWPPLQHILMKGLLNTPATFGKSDPAYQSVQNLALRLAQRYLDSTFCTWYATGGSTSDFPQLEGVTPGATGVMFEKYADNATNVAGGGGEYEVVEGFGWTNGVLIWAADVFGNKLKRPDCGNITAAHTHSSAKRGLEENKLPRRAVELDPWDAAWTKMFGRSKLRRREAEDVRKRWMS

SEQ ID NO: 29 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 30 - DNA, 1644 bp, Penicillium swiecickii
atggcctccactctttctttcaacatttacaaaaacgccctgctctttgctgccttcctaggggctgcgaaggcacagcaagtcggcacaaatacggctgaggttcacccgtctttgacatggcagaagtgtaccgctggaggtacatgcacttcgcaatccggcaaggtcgtcgttgacgcaaactggcgctggcttcacagcaccagtggctccaccaattgctataccggcaatacgtgggatgctactctttgcccagatgatgtgacttgtgccacgaactgcgcgctcgacggtgccgactataccggcacttatggtgttacggctagcggcagctcattgcggttgaacttcgtcaccacagcctctcagaagaacattggctcgcgtctctatcttatggagaatgataccaagtaccaaatgttccagctgctcaaccaggaattcacctttgatgtcgatgtttccaaccttccttgcggcttgaacggcgcgctatacttcgtgtccatggactccgacggaggaatggccagatatcctgccaacaaagccggtgccaagtatggaactggctactgtgattcgcaatgcccgcgtgatctcaagttcatcaacggtcaagctaatgtggagggatgggtgccatcctccaacgatgtcaacgccggcactggcaaccacgggtcttgctgtgcagagatggatatctgggaggccaactcgatctccaatgcggtgactcctcatccatgtgacactccaactcaagtccagtgcaccggagatgcctgtggcggtacatacagctctgaccgctacggtggtacttgtgaccccgacggatgtgatttcaacccgtaccgccagggtaaccagtccttctacggcccgagcaagatcgtggataccacgtctccatttaccgtggtgactcaatttatcaccaatgacggtacctccacgggtactctgtcggagatcaagcgcttctacgtccagaatggcaaggtcatcccccagtcagtatctaccatcagcgctgtgagcggcaactctatcaccgactcctactgcgccgcccagaagaaagccttcggggataccgacgtcttcactaagcatggaggcatggcaggcatgggcgctggtcttgatgaaggcatggttctcgtgatgagtctctgggatgaccacgccgccaatatgctctggctggacagcatataccccacgaccgccacctcgactacgccaggtgctgctcgcggtagctgtgatatttcctctggcaccccgaccgatgttgaagctaaccacgccaacgcctacgtcatctactcgaacatcaaggtcggccctctcggttctaccttcaactcaggtagctctggctctggcactacaaccaccactaccaccaaggccactaccactaccactaccaaggctaccaccaccactactggatcaaccacgaccggtgctgcccactatgcgcaatgcggtggatctagctggactggtgccaccacttgtgccagcccgtacacttgccagaagcagaatgacttctactcccagtgtctgtaggagatgcatctatcagtagcttgatgttatgaccatgtggcg

SEQ ID NO: 31 - DNA, 1397 bp, Cladosporium antareticum
atgaagtcttctttcatcgctgttgcagcggcatcgaccgtcggatctgcgtatgcccaaggtcaggcatatgcccagtgtggaggtgtaggctggtcgggttcgacgagctgtgtctctggatatacctgcaccaagacaaacgactactacagccaatgcctgcccggtggcggcagcaacccaactacgacaactcgtccttctacgacaaccacatctcggactggaggtagccccactggaggctctggctctggccgcgtctcgaatgctggcgtcaacgtgagtaaaccacgaaatacctccaagaacacaactaaccaacaacttccatcagatcgccggcctcgactttggctgcaccatcgacggaacgtgcaagaccgccaacatcgtcgaccccggccagatcggcatcgaccaaatgcagcactttgtcaaagacgacggcttgaacaccttccgcatcccggtcggatggcagttcctgctcaacaacaacctcggcggcaccctcgactccaacaactggaacacgtacgacaagcagatccaaggctgcctgaacaccggcgcagcgctttgcattgttgacattcacaactacgcccgctggaacggtgcgatcgttggccagggtggtccgacgaacgcccagttcaccagcctttggtcgcagatcgcggccaagtataaggggaacaacaagatcgctttcggtctgatgaacgagcccgtaagcgaaacccgaacctcaaccttacgaagaaaacatactgacaagaatcccaccagcacgacctaaacatcaacacctgggcccaaaccgtccaagaagccgtcacggccatccgcaaagccggcgcaaccagcaacaagatcctgctaccgggcaccgacttcacctcggcgggcgccttcatctccaacgggtccggtggcgccctgatcaaagtgaccaacctcgacggcagcacgacgaacctgatcttcgacgtgcacaagtacctcgactcggataactcgggcacgcacacgacgtgcacgaccaacaacgccgaggccttcacgtcgctgggcaactggctgcgcacgaacaagcgccaggcgatcctcaccgagaccgggggtggaccgaatgatgcgagctgcttgaaagctgtgtgcgagcagttggatgtgctgaacagctactctgacgtgtacttggggtggactgggtggagcgcgggccggtttgacgcgacctatgagttgaccgagacgccgacgcgcaatggcaatggcggctggaccgatgtgccgcttgttactcagtgcattgctggcaagttcaagagatagaggggttggggaatcgccggtagatctttgctggatttgtagtccagtcg

SEQ ID NO: 32 - DNA, 2906 bp, Talaromyces pinophilus
atgcggaacagtttattgatttcgcttgctgcggcagcacttgccgagggcaaggtgagtctcttcctcaattgatcccatcatccaatattagaacatgaactaaccgttgcattctacttcaggcatactctcctcccgcataccctgctccctgggccagcggcgccggggaatgggctcaagctcatcaaagagctgtcgagttcgtctcgcaattgaccttggccgaaaaaataaacttgacgaccggtgttgggtatgttgaggaaccattgagcaaatattcactacactggactgacaattggcagctgggagggagggcaatgtgtcggcaacactggaagcattccccgtttgggcttccgcagcctctgcatgcaggattcaccactcggtgtgagagacagtatgtcgtaccccctttacctctttcttcttattttatttcttgaaactagggtccaggatatagctgactgttttactttctccagctgattataatactgccttccctgctggagtcaatgtcgccgctacttgggatctcgatcttgcctaccgacgcggtatagccatggccgaagaacaccgtggcaaaggtgtggacgtgcaacttggtcccgttgctggtccgctaggaagagtaccagagggtggccggaactgggaaggtttcgcgccggaccctgtgttgactggtcagatgatggcaagcactatccaaggcatgcaggataccggtgtgattgcttgcgcaaagcactatattggaaacgaacaagagcacttccgtcagggctctcaggagaacttcaccgttgctgatgctatcagctcgaacattgatgacattactttgcacgaattgtacctgtggccgtttgccgatgcggttagggcaggtgtcggttccgtcatgtgttcttacaatcaaataaataacagctactcctgcggcaacagctacaccctgaaccacatcctcaagggtgaactcgacttccaaggatttgtaatgaccgattggagtgctcaacactctggtgtcggcgatgctcttgccggagctgacatggatatgcctggtgatgtggctttcgacagtggaactgctttctggggtaccaacttgacaattgccgtgctcaatggcactgttcccgaatggcgtgttgacgacatggctgttcgtatcatgtctgctttctacaaggttggtcgtgatcgtacccaggtccccatcaacttcgctagctggaccttggatacctatggcaacgaatactactacgccggcgagggctacaaggaaatcaaccagcacgttgatgtgcgtggtggccacgccaaagtcgtccgtgaaatcggcagtgccagcattgttctcctcaagaacgttgatggtgctcttccattgactggctcggagaagtttgtcgcagtatttggagaggacgcaggctccaaccccgacggtgtcaatggctgctctgaccgtggctgtgataacggtaccttggccatgggatggggtagtggtactgccaacttcccttacttggtcacccctgaacaagctatccaggccgaagtcgtgaagaacggcggtatgtttactgctattaccgacagcggcgccaccgatacagccaagaccgtggctgctcaagcttcgtaagtattcctgctcatgattatacgaatttgctagcttcagatgactgatttgtcttgattagggcttgcctagtgtttgccaatgcagactccggagaaggatacatcaccgttgacggcaatgtgggagatcgtaagaacttgacattgtggcagaacggtgaagctatgatctcggccgtcgcaggtaactgcaacaataccattgtcgttcttcatactgttggacccgttcttgtcgaggactgggtgcaccaccctaacatcactgctgttttgtgggctggtctgcctggagagcagagcggtaactccttggttgatgttctctacggcagtgtcaacccgggaggcaagactcctttcacctggggcaagcaacgttctgactggggagttgatgtcatttacgaacccaacaatggagacggcgctcctcagcaggacttcactgagggtatcttcattgattaccgacactttgacaaatacaacattacccccacctacgaattcggttatggtctcagctacagcaccttctcattctcagacctcaaggtgactcctatcgctgcttctccttaccgaccagccaagggtcagagcggtccggcacctgtgctaggcaaggttttgaacgccacggcctatctgttccctgactacatcaaacgcattgaagcgttcatttacccatggctcaactccactgatcttaagacttcctctggtgatccaaactacggctggccaacttccaagtacgtgcctgacggtgctcaagacgggtctccacaacctgtcaaccccgccggtggtgctcctggtggtaaccctgctctgtatgaccctgttgccgaagtcactgtcaccgtcaagaacactggaaaggtcgctggtgtcgaagtgccccaactctatgtttcgctcggtggtccctccgatgcacccaaggttcttcgtggctttggccgtatttctctcggcgctggcgaggagactcaatggactgccactttgacccgacgtgacgtatctaactgggacactgtcagacaaaactgggttgtcacaaactacaccaagactgtttacgttggcaactcttctcgcaacttgccgctccagcagactttggctctcaacattggacactaataattcggtatggtgattcttttcttgtataaattttggatcggcttccacccttttgt

SEQ ID NO: 33 - DNA, 36 bp, Artificial Sequence
  Artificial Primer
acacaactggggatccaccatggcctccactctttc

SEQ ID NO: 34 - DNA, 39 bp, Artificial Sequence
  Artificial Primer
ccctctagatctcgagcgccacatggtcataacatcaag

SEQ ID NO: 35 - DNA, 43 bp, Artificial Sequence
  Artificial Primer
acacaactggggatccaccatgctgcgatatctttccattgtt

SEQ ID NO: 36 - DNA, 42 bp, Artificial Sequence
  Artificial Primer
ccctctagatctcgagaaaggatattggtgcagtcaaagctg

SEQ ID NO: 37 - DNA, 46 bp, Artificial Sequence
  Artificial Primer
acacaactggggatccaccatgaagtcttctttcatcgctgttgca

SEQ ID NO: 38 - DNA, 48 bp, Artificial Sequence
  Artificial Primer
gtcaccctctagatctcgagcgactggactacaaatccagcaaagatc

SEQ ID NO: 39 - DNA, 43 bp, Artificial Sequence
  Artificial Primer
acacaactggggatccaccatgcggaacagtttattgatttcg

SEQ ID NO: 40 - DNA, 37 bp, Artificial Sequence
  Artificial Primer
gtcaccctctagatctacaaaagggtggaagccgatc

SEQ ID NO: 41 - DNA, 19 bp, Artificial Sequence
  Artificial Primer
tcttttcgcgccctggaaa

SEQ ID NO: 42 - DNA, 37 bp, Artificial Sequence
  Artificial Primer
tttgtttgtttatgtgtgtttattcgaaactaagttc

SEQ ID NO: 43 - DNA, 22 bp, Artificial Sequence
  Artificial Primer
acagaagacgggagacactagc

SEQ ID NO: 44 - DNA, 32 bp, Artificial Sequence
  Artificial Primer
tcagtccaatgacagtattttctccttctcac

SEQ ID NO: 45 - DNA, 25 bp, Artificial Sequence
  Artificial Primer
aacgacagcacaaaggaactttcac

SEQ ID NO: 46 - DNA, 39 bp, Artificial Sequence
  Artificial Primer
ttttattgtatgtgtgtgtgtttgaaactaaagttcttg

SEQ ID NO: 47 - DNA, 31 bp, Artificial Sequence
  Artificial Primer
gccattagtagtgtactcaaacgaattattg

SEQ ID NO: 48 - DNA, 31 bp, Artificial Sequence
  Artificial Primer
ggctactgattttgttaagcaactcatcaag

SEQ ID NO: 49 - DNA, 31 bp, Artificial Sequence; identical to SEQ ID NO: 47
  Artificial Primer

SEQ ID NO: 50 - DNA, 36 bp, Artificial Sequence
  Artificial Primer
gtttagttaattatagttcgttgactgtgtttcttg

SEQ ID NO: 51 - DNA, 28 bp, Artificial Sequence
  Artificial Primer
ggtctcctttctttcaagaaacacagtc

SEQ ID NO: 52 - DNA, 39 bp, Artificial Sequence
  Artificial Primer
atgatgaaaaaataagcagaaaagactaataattcttag

SEQ ID NO: 53 - DNA, 35 bp, Artificial Sequence
  Artificial Primer
agtgcttttaactaagaattattagtcttttctgc

SEQ ID NO: 54 - DNA, 31 bp, Artificial Sequence; identical to SEQ ID NO: 48
  Artificial Primer

SEQ ID NO: 55 - DNA, 28 bp, Artificial Sequence
  Artificial Primer
ggtctcctttctttcaagaaacacagtc

SEQ ID NO: 56 - DNA, 39 bp, Artificial Sequence; identical to SEQ ID NO: 52
  Artificial Primer

SEQ ID NO: 57 - protein, 418 aa, Trichoderma reesei
MNKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 58 - protein, 844 aa, Aspergillus fumigatus
QELAFSPPFYPSPWADGQGEWADAHRRAVEIVSQMTLAEKVNLTTGTGWEMDRCVGQTGSVPRLGINWGLCGQDSPLGIRDSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWGAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTAEFPYLVTPEQAIQREVISNGGNVFAVTDNGALSQMADVASQSSVSLVFVNADSGEGYISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 59 - protein, 405 aa, Evansstolkia leycettana
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 60 - protein, 325 aa, Talaromyces pinophilus
MHFLAALLAVLPLVSGSPVPEKRSGCALPSTYKWTSTGPLASPKSGLVALRDYSHVIYNGQHLVYGSTANTAGSYGSMNFGLFSDWSEMSSASQNTMSTGAVAPTIFYFAPKSVWILAYQWGPYAFSYRTSTDPSNANGWSSPQPLFTGTISGSSTGVIDQTVIGDSENMYLFFAGDNGHIYRASMPIGDFPGSFGSASTIVLSDSTNNLFEAVEVYTVEGQNQYLMIVEAIGANGRYFRSFTASSLGGTWTAQASTESNPFAGKANSGATWTNDISSGDLVRTNPDQTQTIDACNLQFLYQGRSTSSGGDYNLLPYQPGLLTLA

(19) United States
(12) Patent Application Publication
(10) Pub. No.: US 2025/0129393 A1
(43) Pub. Date: Apr. 24, 2025

(54) PROCESSES FOR PRODUCING FERMENTATION PRODUCTS USING FIBER-DEGRADING ENZYMES IN FERMENTATION

(71) Applicant: Novozymes A/S, Bagsvaerd (DK)

(72) Inventors: Jiyin Liu, Raleigh, NC (US); Hui Xu, Wake Forest, NC (US); James Lavigne, Wake Forest, NC (US); Lan Tang, Beijing (CN); Kim Borch, Birkerod (DK); Jesper Frickmann, Raleigh, NC (US); Geoffrey Moxley, Wake Forest, NC (US); Qiming Jin, Sacramento, CA (US); Ye Liu, Beijing (CN)

(73) Assignee: Novozymes A/S, Bagsvard (DK)

(21) Appl. No.: 18/572,135

(22) PCT Filed: Jun. 29, 2022
(86) PCT No.: PCT/CN2022/102201

§ 371 Date: Dec. 19, 2023

(30) Foreign Application Priority Data
Jun. 29, 2021 (WO) PCT/CN2021/103075

(51) Int. Cl. C12P 7/06; C12N 9/26; C12N 9/42; C12N 15/81; C12P 7/10

(52) U.S. Cl. CPC C12P 7/06; C12N 9/2414; C12N 9/2437; C12N 9/2445; C12N 15/81; C12P 7/10; C12Y 302/01001; C12Y 302/01004; C12Y 302/01021; C12Y 302/01091; C12N 2800/102

(57) ABSTRACT

Provided is a subject matter relates to a process for producing fermentation products from starch-containing material comprising the steps of: i) saccharifying using a carbohydrate-source generating enzyme; ii) fermenting using a fermenting organism; wherein at least one polypeptide having cellobiohydrolase activity, endoglucanase activity, or beta-glucosidase activity are present or added during fermentation or simultaneous saccharification and fermentation. The subject matter also relates to an enzyme blend or composition comprising the polypeptides.

### FIELD OF THE INVENTION

The present invention relates to processes for producing fermentation products from starch-containing material. The invention also relates to an enzyme blend or composition, or a recombinant host cell or fermenting organism suitable for use in a process of the invention.

### REFERENCE TO A SEQUENCE LISTING

This application contains a Sequence Listing in computer readable form. The computer readable form is incorporated herein by reference.

### BACKGROUND OF THE INVENTION

Production of fermentation products, such as ethanol, from starch-containing material is well-known in the art. Industrially two different kinds of processes are used today. The most commonly used process, often referred to as a “conventional process”, and includes liquefying gelatinized starch at high temperature using typically a bacterial alpha-amylase, followed by simultaneous saccharification and fermentation carried out in the presence of a glucoamylase and a fermentation organism. Another well-known process, often referred to as a “raw starch hydrolysis”-process (RSH process), includes simultaneously saccharifying and fermenting granular starch below the initial gelatization temperature typically in the presence of at least a glucoamylase.

Despite significant improvement of fermentation product production processes over the past decade a significant amount of residual starch material is not converted into the desired fermentation product, such as ethanol. At least some of the unconverted residual starch material, e.g., sugars and dextrins, is in the form of non-fermentable Maillard products.

Beta-glucans are polysaccharides that only contain glucose as structural components, and in which the glucose units are linked by beta-glycosidic bonds. Cellulose is one type of beta-glucan in which all of the glucose units are linked by beta-1,4-glucosidic bonds. This feature results in the formation of insoluble cellulose micro-fibrils meaning that microbial hydrolysis of cellulose to glucose requires the use of endo-glucanases (EC 3.2.1.4), cellobiohydrolases (EC 3.2.1.91) and beta-glucosidases (EC 3.2.1.21).

Cellulases are well-known for use in the conversion of lignocellulosic feedstocks into ethanol. Once the lignocellulose is converted to fermentable sugars, e.g., glucose, the fermentable sugars are easily fermented by yeast into ethanol. However, there is still a desire and need for providing processes for producing fermentation products, such as ethanol, from starch-containing material that can provide a higher fermentation product yield, or other advantages, compared to a conventional process.

### SUMMARY OF THE INVENTION

Described herein are processes of producing fermentation products, such as ethanol, from starch-containing material using a fermenting organism.

A first aspect relates to a process for producing a fermentation product from starch-containing material, the process comprising the steps of:
i) saccharifying the starch-containing material at a temperature below the initial gelatination temperature; and
ii) fermenting the saccharified starch-containing material using a fermenting organism;
wherein at least one polypeptide having cellobiohydrolase activity, endoglucanase activity, or beta-glucosidase activity is present or added during fermentation.

A second aspect relates to a process for producing a fermentation product from starch-containing material comprising the steps of:
i) liquefying the starch-containing material at a temperature above the initial gelatinization temperature using an alpha-amylase;
ii) saccharifying the liquefied starch-containing;
iii) fermenting saccharified starch-containing material using a fermenting organism;
wherein at least one polypeptide having cellobiohydrolase activity, endoglucanase activity, or beta-glucosidase activity is present or added during fermentation.

A third aspect related to isolated polypeptides having cellobiohydrolase activity, endoglucanase activity, or beta-glucosidase activity.

A fourth aspect relates to an enzyme blend or composition comprising at least one polypeptide having cellobiohydrolase activity, endoglucanase activity, or beta-glucosidase activity.

A fifth aspect relates to isolated polynucleotides encoding the polypeptides having cellobiohydrolase activity, endoglucanase activity, or beta-glucosidase activity; nucleic acid constructs and recombinant expression vectors comprising the polynucleotides; recombinant host cells or fermenting organisms comprising the polynucleotides; and methods of producing the polypeptides having cellobiohydrolase activity, endoglucanase activity, or beta-glucosidase activity.

### BRIEF DESCRIPTION OF THE FIGURES

FIG. 1 shows the final ethanol yield following SSF for control strain MeJi797 and yeast strain S833-E04.

FIG. 2 shows the final ethanol yield following SSF for control strain MeJi797 and strains S1129-C08, S1130-D09 and S11130-H11.

FIG. 3 shows the ethanol yield from synergistic blends of GH10 xylanase and GH62 arabinofuranosidase in simultaneous saccharification and fermentation with a cellulolytic composition.

FIG. 4 shows the ethanol yield from an increasing dose of a hemicelluloytic composition comprising GH10 xylanase and GH62 arabinofuranosidase in simultaneous saccharification and fermentation with a cellulolytic composition.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-amylase: The term “alpha amylase” means an 1,4-alpha-D-glucan glucanohydrolase, EC. 3.2.1.1, which catalyze hydrolysis of starch and other linear and branched 1,4-glucosidic oligo- and polysaccharides. Alpha-amylase activity can be determined using methods known in the art (e.g., using an alpha amylase assay described WO2020/023411).

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinofuranosidase, alpha-arabinofuranosidase, alpha-L-arabinofuranosidase, alpha-arabinofuranosidase, polysaccharide alpha-L-arabinofuranosidase, alpha-L-arabinofuranoside hydrolase, L-arabinofuranosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 micro for 30 minutes at 40 degrees centigrade followed by arabinose analysis by AMINEX® HPX-87H column chromatography (Bio-Rad Laboratories, Inc., Hercules, CA, USA).

Auxiliary Activity 9: The term “Auxiliary Activity 9” or “AA9” means a polypeptide classified as a lytic polysaccharide monooxygenase (Quinlan et al., 2011, Proc. Natl. Acad. Sci. USA 208:15079-15084; Phillips et al., 2011, ACS Chem. Biol. 6:1399-1406; Lin et al., 2012, Structure 20:1051-1061). AA9 polypeptides were formerly classified into the glycoside hydrolase Family 61 (GH61) according to Henrissat, 1991, Biochem. J. 280:309-316, and Henrissat and Bairoch, 1996, Biochem. J. 316:695-696.

AA9 polypeptides enhance the hydrolysis of a cellulosic-containing material by an enzyme having cellulolytic activity. Cellulolytic enhancing activity can be determined by measuring the increase in reducing sugars or the increase of the total of cellobiose and glucose from the hydrolysis of a cellulosic-containing material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40 C-80° C., e.g., 50° C., 55° C., 60° C., 65° C., or 70° C., and a suitable pH, such as 4-9, e.g., 4.5, 5.0, 5.5, 6.0, 6.5, 7.0, 7.5, 8.0, or 8.5, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST® 1.5 L (Novozymes A/S, Bagsværd, Denmark) and beta-glucosidase as the source of the cellulolytic activity, wherein the beta-glucosidase is present at a weight of at least 2-5% protein of the cellulase protein loading. In one embodiment, the beta-glucosidase is an Aspergillus oryzae beta-glucosidase (e.g., recombinantly produced in Aspergillus oryzae according to WO 02/095014). In another embodiment, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic-containing material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42:55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Catalase: The term “catalase” means a hydrogen-peroxide:hydrogen-peroxide oxidoreductase (EC 1.11.1.6) that catalyzes the conversion of 2H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Catalytic domain: The term “catalytic domain” means the region of an enzyme containing the catalytic machinery of the enzyme.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15:160-167; Teeri et al., 1998, Biochem. Soc. Trans. 26:173-178). Cellobiohydrolase activity can be determined according to the procedures described by Lever et al., 1972, Anal. Biochem. 47:273-279; van Tilbeurgh et al., 1982, FEBS Letters 149:152-156; van Tilbeurgh and Claeyssens, 1985, FEBS Letters 187:283-288; and Tomme et al., 1988, Eur. J. Biochem. 170:575-581.

Cellulolytic composition, cellulolytic enzymes or cellulase means a preparation comprising one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24:452-481. Total cellulolytic activity is usually measured using insoluble substrates, including Whatman N^{o}1 filter paper, microcrystalline cellulose, bacterial cellulose, algal cellulose, cotton, pretreated lignocellulose, etc. The most common total cellulolytic activity assay is the filter paper assay using Whatman N^{o}1 filter paper as the substrate. The assay was established by the International Union of Pure and Applied Chemistry (IUPAC) (Ghose, 1987, Measurement of cellulase activities, Pure Appl. Chem. 59:257-68).

Cellulolytic enzyme activity is determined by measuring the increase in hydrolysis of a cellulosic material by cellulolytic enzyme(s) under the following conditions: 1-50 mg of cellulolytic enzyme protein/g of cellulose in Pretreated Corn Stover (“PCS”) (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, CA, USA).

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using from cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)).

Endogenous gene: The term “endogenous gene” means a gene that is native to the referenced host cell. “Endogenous gene expression” means expression of an endogenous gene.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24:452-481). Endoglucanase activity can also be determined using carboxymethyl cellulose (CMC) as substrate according to the procedure of Ghose, 1987, Pure and Appl. Chem. 59:257-268, at pH 5, 40° C.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences that provide for its expression.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. In some instances, the fermentation medium is derived from a natural source, such as sugar cane, starch, or cellulose, and may be the result of pretreating the source by enzymatic hydrolysis (saccharification). The term fermentation medium is understood herein to refer to a medium before the fermenting organism is added, such as, a medium resulting from a saccharification process, as well as a medium used in a simultaneous saccharification and fermentation process (SSF).

Glucoamylase: The term “glucoamylase” (1,4-alpha-D-glucan glucohydrolase, EC 3.2.1.3) is defined as an enzyme that catalyzes the release of D-glucose from the non-reducing ends of starch or related oligo- and polysaccharide molecules. For purposes of the present invention, glucoamylase activity may be determined according to the procedures known in the art, such as those described in WO2020/023411.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59:1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Host cell: The term “host cell” means any cell type that is susceptible to transformation, transfection, transduction, and the like with a nucleic acid construct or expression vector comprising a polynucleotide described herein. The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” is defined herein as a polypeptide having biological activity that is in its final form following translation and any post-translational modifications, such as N-terminal processing, C-terminal truncation, glycosylation, phosphorylation, etc. The mature polypeptide sequence lacks a signal sequence, which may be determined using techniques known in the art (See, e.g., Zhang and Henzel, 2004, Protein Science 13:2819-2824). The term “mature polypeptide coding sequence” means a polynucleotide that encodes a mature polypeptide. In one aspect, the mature polypeptide is amino acids 19 to 520 of SEQ ID NO: 1 based on the SignalP program (Nielsen et al., 1997, Protein Engineering 10:1-6) that predicts amino acids 1 to 18 of SEQ ID NO: 1 are a signal peptide. In another aspect, the mature polypeptide is amino acids 1 to 503 of SEQ ID NO: 2 based on the SignalP program that this sequence lacks a signal peptide. In another aspect, the mature polypeptide is amino acids 26 to 533 of SEQ ID NO: 3 based on the SignalP program that predicts amino acids 1 to 25 of SEQ ID NO: 3 are a signal peptide. In another aspect, the mature polypeptide is amino acids 20 to 456 of SEQ ID NO: 4 based on the SignalP program that predicts amino acids 1 to 19 of SEQ ID NO: 4 are a signal peptide. In another aspect, the mature polypeptide is amino acids 20 to 410 of SEQ ID NO: 5 based on the SignalP program that predicts amino acids 1 to 19 of SEQ ID NO: 5 are a signal peptide. In another aspect, the mature polypeptide is amino acids 20 to 855 of SEQ ID NO: 6 based on the SignalP program that predicts amino acids 1 to 19 of SEQ ID NO: 6 are a signal peptide. It is known in the art that a host cell may produce a mixture of two of more different mature polypeptides (i.e., with a different C-terminal and/or N-terminal amino acid) expressed by the same polynucleotide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, California, including supplements 1-5 published in Eur. J. Biochem. 223:1-5 (1994); Eur. J. Biochem. 232:1-6 (1995); Eur. J. Biochem. 237:1-5 (1996); Eur. J. Biochem. 250:1-6 (1997); and Eur. J. Biochem. 264:610-650 (1999); respectively. The term “subtilases” refer to a sub-group of serine protease according to Siezen et al., 1991, Protein Engng. 4:719-737 and Siezen et al., 1997, Protein Science 6:501-523. Serine proteases or serine peptidases is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Protease activity may be determined using methods described in the art (e.g., US 2015/0125925) or using commercially available assay kits (e.g., Sigma-Aldrich).

Pullulanase: The term “pullulanase” means a starch debranching enzyme having pullulan 6-glucano-hydrolase activity (EC 3.2.1.41) that catalyzes the hydrolysis the α-1,6-glycosidic bonds in pullulan, releasing maltotriose with reducing carbohydrate ends. For purposes of the present invention, pullulanase activity can be determined according to a PHADEBAS assay or the sweet potato starch assay described in WO2016/087237.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Referenced Sequence−Total Number of Gaps in Alignment)

Signal peptide: The term “signal peptide” is defined herein as a peptide linked (fused) in frame to the amino terminus of a polypeptide having biological activity and directs the polypeptide into the cell's secretory pathway. Signal sequences may be determined using techniques known in the art (See, e.g., Zhang and Henzel, 2004, Protein Science 13:2819-2824). The polypeptides described herein may comprise any suitable signal peptide known in the art, or any signal peptide described in WO2021/025872 (incorporated herein by reference).

Trehalase: The term “trehalase” means an enzyme which degrades trehalose into its unit monosaccharides (i.e., glucose). Trehalases are classified in EC 3.2.1.28 (alpha, alpha-trehalase) and EC. 3.2.1.93 (alpha, alpha-phosphotrehalase). The EC classes are based on recommendations of the Nomenclature Committee of the International Union of Biochemistry and Molecular Biology (IUBMB). Description of EC classes can be found on the internet, e.g., on “http://www.expasy.org/enzyme/”. Trehalases are enzymes that catalyze the following reactions:

Alpha,alpha-trehalose+H₂O⇔2 D-glucose; EC 3.2.1.28:

Alpha,alpha-trehalose 6-phosphate+H2O⇔D-glucose+D-glucose 6-phosphate. EC 3.2.1. 93:

Trehalase activity may be determined according to procedures known in the art.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### DETAILED DESCRIPTION OF THE INVENTION

The present invention relates to processes of producing fermentation products, such as ethanol from starch-containing material using a fermenting organism.

Processes for Producing Fermentation Products from Un-Gelatinized Starch-Containing Material.

Described herein are processes for producing fermentation products from starch-containing material without gelatinization (i.e., without cooking) of the starch-containing material (often referred to as a “raw starch hydrolysis” process). The fermentation product, such as ethanol, can be produced without liquefying the aqueous slurry containing the starch-containing material and water. In one embodiment a process of the invention includes saccharifying (e.g., milled) starch-containing material, e.g., granular starch, below the initial gelatinization temperature, preferably in the presence of an alpha-amylase and carbohydrate-source generating enzyme(s) to produce sugars that can be fermented into the fermentation product by a suitable fermenting organism. In this embodiment the desired fermentation product, e.g., ethanol, is produced from un-gelatinized (i.e., uncooked), preferably milled, cereal grains, such as corn.

Processes for producing a fermentation product from starch-containing material may comprise simultaneously saccharifying and fermenting starch-containing material using a carbohydrate-source generating enzymes and a fermenting organism at a temperature below the initial gelatinization temperature of said starch-containing material in the presence of an alpha-amylase of the invention. Saccharification and fermentation may also be separate.

One aspect relates to processes for producing fermentation products, such as ethanol, from starch-containing material, the pro comprising the steps of:
i) saccharifying the starch-containing material at a temperature below the initial gelatination temperature; and
ii) fermenting the saccharified starch-containing material using a fermenting organism;
wherein at least one polypeptide having cellobiohydrolase activity, endoglucanase activity, or beta-glucosidase activity is present or added during fermentation.

The at least one polypeptide having cellobiohydrolase activity, endoglucanase activity, or beta-glucosidase activity present or added in the above described processes for producing fermentation products from starch-containing material may be added exogenously during saccharification, fermentation or simultaneous saccharification and fermentation as mono-components, as enzyme blends or compositions comprising the polypeptide having cellobiohydrolyase activity, endoglucanase activity, or beta-glucosidase activity, and/or via in-situ expression and secretion of the polypeptide having cellobiohydrolyase activity, endoglucanase activity, or beta-glucosidase activity by the fermenting organism, e.g., a recombinant host cell or fermenting organism described herein (e.g., yeast, such as from the genus Saccharomyces, preferably Saccharomyces cerevisiae).

Processes for Producing Fermentation Products from Gelatinized Starch-Containing Material

Also described are processes for producing fermentation products, such as ethanol, from starch-containing material, which process includes a liquefaction step and sequentially or simultaneously performed saccharification and fermentation steps.

One aspect relates to processes for producing fermentation products from starch-containing material, the process comprising the steps of:
i) liquefying the starch-containing material at a temperature above the initial gelatinization temperature using an alpha-amylase;
ii) saccharifying the liquefied starch-containing;
iii) fermenting saccharified starch-containing material using a fermenting organism;
wherein at least one polypeptide having cellobiohydrolase activity, endoglucanase activity, or beta-glucosidase activity is present or added during fermentation.

The at least one polypeptide having cellobiohydrolase activity, endoglucanase activity, or beta-glucosidase activity present or added in the above described processes for producing fermentation products from starch-containing material may be added exogenously during saccharification, fermentation or simultaneous saccharification and fermentation as mono-components, as enzyme blends or compositions comprising the polypeptide having cellobiohydrolyase activity, endoglucanase activity, or beta-glucosidase activity, and/or via in-situ expression and secretion of the polypeptide having cellobiohydrolyase activity, endoglucanase activity, or beta-glucosidase activity by the fermenting organism, e.g., a recombinant host cell or fermenting organism described herein (e.g., yeast, such as from the genus Saccharomyces, preferably Saccharomyces cerevisiae).

Steps ii) and iii) may be carried out either sequentially or simultaneously. In a preferred embodiment steps ii) and iii) are carried out simultaneously. The alpha-amylase, an optional thermostable protease, and/or an optional thermostable xylanase, may be added before and/or during liquefaction step i).

A composition of the invention may suitably be used in a process of the invention. A recombinant host cell or fermenting organism of the invention may suitably be used in a process of the invention. However, the enzymes may also be added separately.

Whether the process includes a liquefaction step or not, the essential feature of the invention is that at least one polypeptide having cellobiohydrolase activity, endoglucanase activity, or beta-glucosidase activity are present or added during fermentation or simultaneous saccharification and fermentation. In one embodiment, at least one polypeptide having cellobiohydrolase activity, endoglucanase activity, or beta-glucosidase activity are present or added during fermentation or simultaneous saccharification and fermentation. In another embodiment, as noted above, the at least one polypeptide having cellobiohydrolase activity, endoglucanase activity, or beta-glucosidase activity may be added exogenously as a standalone enzyme or an enzyme blend or composition comprising at least one, at least two, at least three, at least four, or at least five polypeptides having cellobiohydrolase activity, endoglucanase activity, or beta-glucosidase activity, or expressed and secreted in situ by a recominbant host cell or fermenting organism. In an example embodiment, the at least one polypeptide having cellobiohydrolase activity, endoglucanase activity, or beta-glucosidase activity is added in the form of a cellulolytic composition that comprises the at least one polypeptide and the at least one additional cellulase. In an embodiment, the cellulolytic composition comprises at least two polypeptides having cellobiohydrolase activity, endoglucanase activity, or beta-glucosidase activity. In an embodiment, the cellulolytic composition comprises at least three polypeptides having cellobiohydrolase activity, endoglucanase activity, or beta-glucosidase activity. In an embodiment, the cellulolytic composition comprises a polypeptide having cellobiohydrolase activity, a polypeptide having endoglucanase activity, and a polypeptide having beta-glucosidase activity. Any cellullytic composition described herein can be present or added during fermentation or simultaneous saccharification and fermentation.

Any cellulase having cellobiohydrolase activity, endoglucanase activity, or beta-glucosidase activity can be present and/or added during saccharification, fermentation, or SSF in a process of the invention (e.g., any mature polypeptide of SEQ ID NO: 1, SEQ ID NO: 2, SEQ ID NO: 3, SEQ ID NO: 4, SEQ ID NO: 5, SEQ ID NO: 6, or variants thereof).

Exemplary cellulases that may be used with the processes described herein and/or expressed by the host cells or fermenting organisms described herein include, but are not limited to the cellulases shown in Table 1 (or derivatives thereof).

TABLE 1

| | | SEQ |
|---|---|---|
| | | ID |
| Donor Organism | Enzyme class | NO. |
| Penicillium emersonii | Cellobiohydrolase 1 | 1 |
| Aspergillus nidulans | Cellobiohydrolase 1 | 2 |
| Penicillium swiecickii | Cellobiohydrolase 1 | 3 |
| Talaromyces verruculosus | Cellobiohydrolase 2 | 4 |
| Cladosporium antareticum | Endoglucanase | 5 |
| Talaromyces pinophilus | beta-glucosidase | 6 |

The polypeptide having cellobiohydrolase activity, endoglucanase activity, or beta-glucosidase activity may be obtained from microorganisms of any genus. For purposes herein, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In one embodiment, the polypeptide having cellobiohydrolase activity is a Penicillium cellobiohydrolase I. In another embodiment, the polypeptide having cellobiohydrolase activity is a Penicillium emersonii cellobiohydrolase I. In one embodiment, the polypeptide having cellobiohydrolase activity is an Aspergillus cellobiohydrolase I. In another embodiment, the polypeptide having cellobiohydrolase activity is an Aspergillus nidulans cellobiohydrolase I. In one embodiment, the polypeptide having cellobiohydrolase activity is a Penicillium cellobiohydrolase I. In another embodiment, the polypeptide having cellobiohydrolase activity is a Penicillium swiecickii cellobiohydrolase I. In one embodiment, the polypeptide having cellobiohydrolase activity is a Talaromyces cellobiohydrolase II. In another embodiment, the polypeptide having cellobiohydrolase activity is a Talaromyces verruculosus cellobiohydrolase II. In one embodiment, the polypeptide having endoglucanase activity is a Cladosporium endoglucanase. In another embodiment, the polypeptide having endoglucanase activity is a Cladosporium antareticum endoglucanase. In one embodiment, the polypeptide having beta-glucosidase activity is a Talaromyces beta-glucosidase. In another embodiment, the polypeptide having beta-glucosidase activity is a Talaromyces pinophilus beta-glucosidase. It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide having cellobiohydrolase activity, endoglucanase activity, or beta-glucosidase activity coding sequences described or referenced herein, or a subsequence thereof, as well as the transporter described or referenced herein, or a fragment thereof, may be used to design nucleic acid probes to identify and clone DNA encoding a glycerol transporter from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a sugar transporter. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with a coding sequence, or a subsequence thereof, the carrier material is used in a Southern blot.

In one embodiment, the nucleic acid probe is a polynucleotide, or subsequence thereof, that encodes the mature cellobiohydrolase of SEQ ID NO: 1, 2, 3 or 4; the mature endoglucanase of SEQ ID NO: 5, or the mature beta-glucosidase of SEQ ID NO: 6, or a fragment thereof.

For purposes of the probes described above, hybridization indicates that the polynucleotide hybridizes to a labeled nucleic acid probe, or the full-length complementary strand thereof, or a subsequence of the foregoing; under very low to very high stringency conditions. Molecules to which the nucleic acid probe hybridizes under these conditions can be detected using, for example, X-ray film. Stringency and washing conditions are defined as described supra.

In one embodiment, the polypeptide having cellobiohydrolase activity, endoglucanase activity, or beta-glucosidase activity is encoded by a polynucleotide that hybridizes under at least low stringency conditions, e.g., medium stringency conditions, medium-high stringency conditions, high stringency conditions, or very high stringency conditions with the full-length complementary strand of the coding sequence for any one of the glycerol transporters described or referenced herein (e.g., SEQ ID NOs: 312-323). (Sambrook et al., 1989, Molecular Cloning, A Laboratory Manual, 2d edition, Cold Spring Harbor, New York).

The polypeptide having cellobiohydrolase activity, endoglucanase activity, or beta-glucosidase activity may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding a glycerol transporter may then be derived by similarly screening a genomic or cDNA library of another microorganism or mixed DNA sample.

Once a polynucleotide encoding a polypeptide having cellobiohydrolase activity, endoglucanase activity, or beta-glucosidase activity has been detected with a suitable probe as described herein, the sequence may be isolated or cloned by utilizing techniques that are known to those of ordinary skill in the art (See, e.g., Sambrook et al., 1989, supra). Techniques used to isolate or clone polynucleotides encoding polypeptides include isolation from genomic DNA, preparation from cDNA, or a combination thereof. The cloning of the polynucleotides from such genomic DNA can be affected, e.g., by using the well-known polymerase chain reaction (PCR) or antibody screening of expression libraries to detect cloned DNA fragments with shares structural features (See, e.g., Innis et al., 1990, PCR: A Guide to Methods and Application, Academic Press, New York). Other nucleic acid amplification procedures such as ligase chain reaction (LCR), ligated activated transcription (LAT) and nucleotide sequence-based amplification (NASBA) may be used.

In one embodiment, the polypeptide having cellobiohydrolase activity comprises or consists of the amino acid sequence of SEQ ID NO: 1, 2, 3, 4, or mature polypeptide thereof. In another embodiment, the polypeptide having cellobiohydrolase is a fragment of the cellobiohydrolase of SEQ ID NO: 1, 2, 3, or 4, or the mature polypeptide thereof, wherein, e.g., the fragment has cellobiohydrolase activity. In one embodiment, the number of amino acid residues in the fragment is at least 75%, e.g., at least 80%, 85%, 90%, or 95% of the number of amino acid residues in referenced full length cellobiohydrolase (e.g. any one of SEQ ID NOs: 1, 2, 3, or 4, or the mature polypeptide thereof). In other embodiments, the polypeptide having cellobiohydrolase activity may comprise the catalytic domain of any cellobiohydrolase described or referenced herein (e.g., the catalytic domain of SEQ ID NO: 1, 2, 3, or 4).

The polypeptide having cellobiohydrolase activity may be a variant of any one of the cellobiohydrolases described supra (e.g., any one of SEQ ID NOs: 1, 2, 3, or 4, or the mature polypeptide thereof). In one embodiment, the polypeptide having cellobiohydrolase activity has at least 60%, e.g., at least 65%, 70%, 75%, 80%, 85%, 90%, 95%, 97%, 98%, 99%, or 100% sequence identity to the amino acid sequence of SEQ ID NO: 1 or the mature polypeptide thereof. In one embodiment, the polypeptide having cellobiohydrolase activity has at least 60%, e.g., at least 65%, 70%, 75%, 80%, 85%, 90%, 95%, 97%, 98%, 99%, or 100% sequence identity to the amino acid sequence of SEQ ID NO: 2, or the mature polypeptide thereof. In one embodiment, the polypeptide having cellobiohydrolase activity has at least 60%, e.g., at least 65%, 70%, 75%, 80%, 85%, 90%, 95%, 97%, 98%, 99%, or 100% sequence identity to the amino acid sequence of SEQ ID NO: 3, or mature polypeptide thereof. In one embodiment, the polypeptide having cellobiohydrolase activity has at least 60%, e.g., at least 65%, 70%, 75%, 80%, 85%, 90%, 95%, 97%, 98%, 99%, or 100% sequence identity to the amino acid sequence of SEQ ID NO: 4, or mature polypeptide thereof.

In one embodiment, the sequence of the polypeptide having cellobiohydrolase activity differs by no more than ten amino acids, e.g., by no more than five amino acids, by no more than four amino acids, by no more than three amino acids, by no more than two amino acids, or by one amino acid from the amino acid sequence of any one of the cellobiohydrolases described supra (e.g., any one of SEQ ID NOs: 1, 2, 3, or 4, or mature polypeptide thereof). In one embodiment, the polypeptide having cellobiohydrolase activity has an amino acid substitution, deletion, and/or insertion of one or more (e.g., two, several) of the amino acid sequence of any one of the cellobiohydrolases described supra (e.g., any one of SEQ ID NOs: 1, 2, 3, or 4, or mature polypeptide thereof). In some embodiments, the total number of amino acid substitutions, deletions and/or insertions is not more than 10, e.g., not more than 9, 8, 7, 6, 5, 4, 3, 2, or 1.

In one embodiment, the polypeptide having endoglucanase activity comprises or consists of the amino acid sequence of SEQ ID NO: 5, or mature polypeptide thereof. In another embodiment, the polypeptide having endoglucanase is a fragment of the endoglucanase of SEQ ID NO: 5, or mature polypeptide thereof, wherein, e.g., the fragment has endoglucanase activity. In one embodiment, the number of amino acid residues in the fragment is at least 75%, e.g., at least 80%, 85%, 90%, or 95% of the number of amino acid residues in referenced full length endoglucanase (e.g. of SEQ ID NO: 5, or mature polypeptide thereof). In other embodiments, the polypeptide having endoglucanase activity may comprise the catalytic domain of any endoglucanase described or referenced herein (e.g., the catalytic domain of SEQ ID NO: 5).

The polypeptide having endoglucanase activity may be a variant of the endoglucanase of SEQ ID NO: 5. In one embodiment, the polypeptide having endoglucanase activity has at least 60%, e.g., at least 65%, 70%, 75%, 80%, 85%, 90%, 95%, 97%, 98%, 99%, or 100% sequence identity to the amino acid sequence of SEQ ID NO: 5, or mature polypeptide thereof.

In one embodiment, the sequence of the polypeptide having endoglucanase activity differs by no more than ten amino acids, e.g., by no more than five amino acids, by no more than four amino acids, by no more than three amino acids, by no more than two amino acids, or by one amino acid from the amino acid sequence SEQ ID NO: 5, or mature polypeptide thereof. In one embodiment, the polypeptide having endoglucanase activity has an amino acid substitution, deletion, and/or insertion of one or more (e.g., two, several) of the amino acid sequence of SEQ ID NO: 5, or mature polypeptide thereof. In some embodiments, the total number of amino acid substitutions, deletions and/or insertions is not more than 10, e.g., not more than 9, 8, 7, 6, 5, 4, 3, 2, or 1.

In one embodiment, the polypeptide having beta-glucosidase activity comprises or consists of the amino acid sequence of SEQ ID NO: 6, or mature polypeptide thereof. In another embodiment, the polypeptide having beta-glucosidase is a fragment of the beta-glucosidase of SEQ ID NO: 6, or mature polypeptide thereof, wherein, e.g., the fragment has beta-glucosidase activity. In one embodiment, the number of amino acid residues in the fragment is at least 75%, e.g., at least 80%, 85%, 90%, or 95% of the number of amino acid residues in referenced full length endoglucanase (e.g. of SEQ ID NO: 6, or mature polypeptide thereof). In other embodiments, the polypeptide having beta-glucosidase activity may comprise the catalytic domain of any beta-glucosidase described or referenced herein (e.g., the catalytic domain of SEQ ID NO: 6, or mature polypeptide thereof).

The polypeptide having beta-glucosidase activity may be a variant of the beta-glucosidase of SEQ ID NO: 6. In one embodiment, the polypeptide having beta-glucosidase activity has at least 60%, e.g., at least 65%, 70%, 75%, 80%, 85%, 90%, 95%, 97%, 98%, 99%, or 100% sequence identity to the amino acid sequence of SEQ ID NO: 6, or mature polypeptide thereof.

In one embodiment, the sequence of the polypeptide having beta-glucosidase activity differs by no more than ten amino acids, e.g., by no more than five amino acids, by no more than four amino acids, by no more than three amino acids, by no more than two amino acids, or by one amino acid from the amino acid sequence SEQ ID NO: 6, or mature polypeptide thereof. In one embodiment, the polypeptide having beta-glucosidase activity has an amino acid substitution, deletion, and/or insertion of one or more (e.g., two, several) of the amino acid sequence of SEQ ID NO: 6, or mature polypeptide thereof. In some embodiments, the total number of amino acid substitutions, deletions and/or insertions is not more than 10, e.g., not more than 9, 8, 7, 6, 5, 4, 3, 2, or 1.

The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the glycerol transporters, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271:4699-4708. The active site or other biological interaction can also be determined by physical analysis of structure, as determined by such techniques as nuclear magnetic resonance, crystallography, electron diffraction, or photoaffinity labeling, in conjunction with mutation of putative contact site amino acids (See, for example, de Vos et al., 1992, Science 255:306-312; Smith et al., 1992, J. Mol. Biol. 224:899-904; Wlodaver et al., 1992, FEBS Lett. 309:59-64). The identities of essential amino acids can also be inferred from analysis of identities with other cellulases that are related to the referenced glycerol transporter.

Additional guidance on the structure-activity relationship of the cellulases herein can be determined using multiple sequence alignment (MSA) techniques well-known in the art. Based on the teachings herein, the skilled artisan could make similar alignments with any number of cellulases described herein or known in the art. Such alignments aid the skilled artisan to determine potentially relevant domains (e.g., binding domains or catalytic domains), as well as which amino acid residues are conserved and not conserved among the different cellulase sequences. It is appreciated in the art that changing an amino acid that is conserved at a particular position between disclosed polypeptides will more likely result in a change in biological activity (Bowie et al., 1990, Science 247:1306-1310: “Residues that are directly involved in protein functions such as binding or catalysis will certainly be among the most conserved”). In contrast, substituting an amino acid that is not highly conserved among the polypeptides will not likely or significantly alter the biological activity.

Even further guidance on the structure-activity relationship for the skilled artisan can be found in published x-ray crystallography studies known in the art.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO95/17413; or WO95/22625. Other methods that can be used include error-prone PCR, phage display (e.g., Lowman et al., 1991, Biochemistry 30:10832-10837; U.S. Pat. No. 5,223,409; WO92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active glycerol transporters can be recovered from the host cells and rapidly sequenced using standard methods in the art. These methods allow the rapid determination of the importance of individual amino acid residues in a polypeptide.

In one embodiment, the heterologous polynucleotide encoding the polypeptide having cellobiohydrolase activity comprises or consists of a coding sequence of any one of the cellobiohydrolases described supra (e.g., any one of SEQ ID NOs: 1, 2, 3, or 4, or mature polypeptide thereof). In another embodiment, the heterologous polynucleotide encoding the polypeptide having cellobiohydrolase activity comprises a subsequence of a coding sequence of any one of the cellobiohydrolases described supra (e.g., any one of SEQ ID NOs: 1, 2, 3, or 4, or mature polypeptide thereof) wherein the subsequence encodes a polypeptide having cellobiohydrolase activity. In another embodiment, the number of nucleotides residues in the coding subsequence is at least 75%, e.g., at least 80%, 85%, 90%, or 95% of the number of the referenced coding sequence.

In another embodiment, the heterologous polynucleotide encoding the polypeptide having cellobiohydrolase activity comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to a coding sequence of any one of the cellobiohydrolases described supra (e.g., any one of SEQ ID NOs: 1, 2, 3, or 4, or mature polypeptide thereof).

In one embodiment, the heterologous polynucleotide encoding the polypeptide having endoglucanase activity comprises or consists of the coding sequence of the endoglucanase of SEQ ID NO: 5. In another embodiment, the heterologous polynucleotide encoding the polypeptide having endoglucanase activity comprises a subsequence of a coding sequence of the endoglucanase of SEQ ID NO: 5 wherein the subsequence encodes a polypeptide having endoglucanase activity. In another embodiment, the number of nucleotides residues in the coding subsequence is at least 75%, e.g., at least 80%, 85%, 90%, or 95% of the number of the referenced coding sequence.

In another embodiment, the heterologous polynucleotide encoding the polypeptide having endoglucanase activity comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to a coding sequence of the endoglucanase of SEQ ID NO: 5, or mature polypeptide thereof.

In one embodiment, the heterologous polynucleotide encoding the polypeptide having beta-glucosidase activity comprises or consists of a coding sequence of the beta-glucosidase of SEQ ID NO: 6, or mature polypeptide thereof. In another embodiment, the heterologous polynucleotide encoding the polypeptide having beta-glucosidase activity comprises a subsequence of a coding sequence of the beta-glucosidase of SEQ ID NO: 6, or mature polypeptide thereof wherein the subsequence encodes a polypeptide having beta-glucosidase activity. In another embodiment, the number of nucleotides residues in the coding subsequence is at least 75%, e.g., at least 80%, 85%, 90%, or 95% of the number of the referenced coding sequence.

In another embodiment, the heterologous polynucleotide encoding the polypeptide having beta-glucosidase activity comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to a coding sequence of the beta-glucosidase of SEQ ID NO: 6, or mature polypeptide thereof.

The referenced coding sequence of any related aspect or embodiment described herein can be the native coding sequence or a degenerate sequence, such as a codon-optimized coding sequence designed for use in a particular host cell (e.g., optimized for expression in Saccharomyces cerevisiae or any other host used for production). Codon-optimization for expression in yeast cells is known in the art (e.g., U.S. Pat. No. 8,326,547).

The polypeptide having cellobiohydrolase activity, endoglucanase activity, or beta-glucosidase activity may be a fused polypeptide or cleavable fusion polypeptide in which another polypeptide is fused at the N-terminus or the C-terminus of the glycerol transporter. A fused polypeptide may be produced by fusing a polynucleotide encoding another polypeptide to a polynucleotide encoding the glycerol transporter. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12:2575-2583; Dawson et al., 1994, Science 266:776-779).

In some embodiments, the polypeptide having cellobiohydrolase activity, endoglucanase activity, or beta-glucosidase activity is a fusion protein comprising a signal peptide linked to the N-terminus of polypeptide, such as any signal sequences described in WO2021/025872 “Fusion Proteins For Improved Enzyme Expression” (the content of which is hereby incorporated by reference).

In terms of dose ranges envisaged, in one embodiment, the polypeptide having cellobiohydrolase activity, endoglucanase activity, and/or beta-glucosidase activity are dosed in the range 0.1-1000 micro gram EP/g DS; 0.5-500 micro gram EP/g DS; 1-100 micro gram EP/g DS; such as 5-50 micro gram EP/g DS.

As described supra, the polypeptides having cellobiohydrolase activity, endoglucanase activity, or beta-glucosidase activity are present or added during fermentation or simultaneous saccharification and fermentation, however, preferred embodiments may also include the addition of other enzyme classes during fermentation/SSF. Examples of other enzymes that can be added during fermentation/SSF include, without limitation, alpha-amylases, glucoamylases, trehalases, cellulases/cellulolytic compositions, and hemicellulases/hemicellulolytic compositions. Particularly, saccharification and/or fermentation or simultaneous saccharification and fermentation, is performed in the presence of at least one cellulase/cellulolytic composition. More particularly the cellulases/cellulolytic composition are derived from a strain of Trichoderma, in particular Trichoderma reesei, or a strain of Humicola, in particular Humicola insolens, or a strain of Chrysosporium, in particular Chrysosporium lucknowense. The cellulases/cellulolytic composition should at least comprise a beta-glucosidase, a cellobiohydrolase and an endoglucanase. In one embodiment, the cellulases/cellulolytic composition comprises one or more polypeptides selected from the group consisting of:
GH61 polypeptide having cellulolytic enhancing activity,
beta-glucosidase;
Cellobiohydrolase I;
Cellobiohydrolase II;
   or a mixture of two, three, or four thereof.

Cellulases are well known in the art, and many are derived from filamentous fungi. Particularly, according to the invention, the cellulases/cellulolytic composition comprises one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

More specifically the cellulases/cellulolytic composition is in one embodiment a Trichoderma reesei cellulolytic enzyme composition further comprising Penicillium emersonii GH61A polypeptide having cellulolytic enhancing activity disclosed in SEQ ID NO: 7, or polypeptide having at least 90% identity, at least 95% identity, at least 96% identity, at least 97% identity, at least 99% identity to SEQ ID NO: 7 and an Aspergillus fumigatus beta-glucosidase disclosed in SEQ ID NO: 8 or a variant thereof with the following substitutions: F100D, S283G, N456E, F512Y having at least 90% identity, at least 95% identity, at least 96% identity, at least 97% identity, at least 99% identity to SEQ ID NO: 8.

In one embodiment, the cellulolytic composition comprises a cellobiohydrolase I (CBH I), such as one derived from a strain of the genus Aspergillus, such as a strain of Aspergillus fumigatus, such as the CBHI disclosed as SEQ ID NO: 9, or CBH I having at least 90% identity, at least 95% identity, at least 96% identity, at least 97% identity, at least 99% identity to SEQ ID NO: 9.

In one embodiment, the cellulolytic composition comprises a cellobiohydrolase II (CBH II), such as one derived from a strain of the genus Aspergillus, such as a strain of Aspergillus fumigatus; such as the CBH II disclosed as SEQ ID NO: 10, or a CBH II having at least 90% identity, at least 95% identity, at least 96% identity, at least 97% identity, at least 99% identity to SEQ ID NO: 10.

Examples of suitable cellulases can be found in “Cellulolytic Composition present and/or added during Saccharification and/or Fermentation”.

Saccharification and/or fermentation or simultaneous saccharification and fermentation can be performed in the presence of at least one hemicellulolytic composition. In an embodiment, the hemicellulolytic composition present or added during saccharification and/or fermentation or simultaneous saccharification and fermentation comprises: (i) a GH10 xylanase; and (ii) a GH62 arabinofuranosidase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of beta-glucosidase, cellobiohydrolase, and endoglucanase; and wherein the hemicellulolytic composition comprises an arabinofuranosidase and a xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises an arabinofuranosidase and a xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises a GH62 arabinofuranosidase and a GH10 xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises a Talaromyces GH62 arabinofuranosidase and a Talaromyces GH10 xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises a Talaromyces pinophilus GH62 arabinofuranosidase and a Talaromyces leycettanus GH10 xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises: (i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and (ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of an Aspergillus beta-glucosidase or a variant thereof; an Aspergillus cellobiohydrolase I; and a Trichoderma endoglucanase I; and wherein the hemicellulolytic composition comprises an arabinofuranosidase and a xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of an Aspergillus beta-glucosidase or a variant thereof; an Aspergillus cellobiohydrolase I; and a Trichoderma endoglucanase I; and wherein the hemicellulolytic composition comprises an arabinofuranosidase and a xylanase, wherein the cellulolytic composition is derived from a strain of Trichoderma, such as Trichoderma reesei, or a strain of Humicola, such as Humicola insolens, or a strain of Chrysosporium, such as Chrysosporium lucknowense.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of an Aspergillus fumigatus beta-glucosidase or a variant thereof; an Aspergillus fumigatus cellobiohydrolase I; and a Trichoderma reesei endoglucanase I; and wherein the hemicellulolytic composition comprises an arabinofuranosidase and a xylanase.

In an example preferred embodiment, saccharification and/or fermentation or simultaneous saccharification and fermentation is performed in the presence of a cellulolytic composition and a hemicellulolytic composition, wherein the cellulolytic composition comprises two or three polypeptides selected from the group consisting of an Aspergillus fumigatus beta-glucosidase or a variant thereof; an Aspergillus fumigatus cellobiohydrolase I; and a Trichoderma reesei endoglucanase I; and wherein the hemicellulolytic composition comprises an arabinofuranosidase and a xylanase, wherein the cellulolytic composition is derived from a strain of Trichoderma, such as Trichoderma reesei, or a strain of Humicola, such as Humicola insolens, or a strain of Chrysosporium, such as Chrysosporium lucknowense.

In an example preferred embodiment, saccharification and/or fermentation or simultaneous saccharification and fermentation is performed in the presence of a cellulolytic composition and a hemicellulolytic composition, wherein the cellulolytic composition comprises:
(i) an Aspergillus fumigatus beta-glucosidase disclosed in SEQ ID NO: 8 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 8;
(ii) a cellobiohydrolase I (CBH I), such as one derived from a strain of the genus Aspergillus, such as a strain of Aspergillus fumigatus, such as the CBHI disclosed as SEQ ID NO: 9, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 9; and
(iii) an endoglucanase I (EGI), such as one derived from a strain of the genus Trichoderma, such as a strain of Trichoderma reesei, such as the EGI disclosed as SEQ ID NO: 29, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 29; and
wherein the hemicellulolytic composition comprises an arabinofuranosidase and a xylanase.

In an example preferred embodiment, saccharification and/or fermentation or simultaneous saccharification and fermentation is performed in the presence of a cellulolytic composition and a hemicellulolytic composition, wherein the cellulolytic composition comprises an Aspergillus beta-glucosidase or a variant thereof; an Aspergillus cellobiohydrolase I; and a Trichoderma endoglucanase I; and wherein the hemicellulolytic composition comprises an GH10 arabinofuranosidase and a GH10 xylanase.

In an example preferred embodiment, saccharification and/or fermentation or simultaneous saccharification and fermentation is performed in the presence of a cellulolytic composition and a hemicellulolytic composition, wherein the cellulolytic composition comprises an Aspergillus beta-glucosidase or a variant thereof; an Aspergillus cellobiohydrolase I; and a Trichoderma endoglucanase I; and wherein the hemicellulolytic composition comprises a Talaromyces GH10 arabinofuranosidase and a Talaromyces GH10 xylanase.

In an example preferred embodiment, saccharification and/or fermentation or simultaneous saccharification and fermentation is performed in the presence of a cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises an GH10 arabinofuranosidase and a GH10 xylanase.

In an example preferred embodiment, saccharification and/or fermentation or simultaneous saccharification and fermentation is performed in the presence of a cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises a Talaromyces pinophilus GH10 arabinofuranosidase and a Talaromyces leycettanus GH10 xylanase.

In an example preferred embodiment, saccharification and/or fermentation or simultaneous saccharification and fermentation is performed in the presence of a Trichoderma cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises a Talaromyces pinophilus GH10 arabinofuranosidase and a Talaromyces leycettanus GH10 xylanase.

In an example preferred embodiment, saccharification and/or fermentation or simultaneous saccharification and fermentation is performed in the presence of a Trichoderma reesei cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises a Talaromyces pinophilus GH10 arabinofuranosidase and a Talaromyces leycettanus GH10 xylanase.

In an example preferred embodiment, saccharification and/or fermentation or simultaneous saccharification and fermentation is performed in the presence of a Trichoderma reesei cellulolytic composition and a hemicellulolytic composition, wherein the cellulolytic composition comprises:
(i) an Aspergillus fumigatus beta-glucosidase disclosed in SEQ ID NO: 8 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 8;
(ii) a cellobiohydrolase I (CBH I), such as one derived from a strain of the genus Aspergillus, such as a strain of Aspergillus fumigatus, such as the CBHI disclosed as SEQ ID NO: 9, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 9; and
(iii) an endoglucanase I (EGI), such as one derived from a strain of the genus Trichoderma, such as a strain of Trichoderma reesei, such as the EGI disclosed as SEQ ID NO: 29, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 29; and
wherein the hemicellulolytic composition comprises:
(i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and (ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

Examples of alpha-amylases can be found in the “Alpha-Amylase Present and/or Added During Liquefaction”-section below. Examples of thermostable proteases can be found in the “Protease Present and/or Added During Liquefaction”-section below. Examples of suitable optional carbohydrate-source generating enzymes, preferably thermostable carbohydrate-source generating enzymes, in particular, a thermostable glucoamylase, can be found in the “Carbohydrate-Source Generating Enzymes Present and/or Added During Liquefaction”-section below.

The pH during liquefaction may be between 4-7. In an embodiment the pH during liquefaction is from 4.5-5.0, such as between 4.5-4.8. In another embodiment liquefaction is carried out at a pH above 5.0-6.5, such as above 5.0-6.0, such as above 5.0-5.5, such as between 5.2-6.2, such as around 5.2, such as around 5.4, such as around 5.6, such as around 5.8.

The process temperature during liquefaction is above the initial gelatinization temperature. The term “initial gelatinization temperature” refers to the lowest temperature at which solubilization of starch, typically by heating, begins. The temperature can vary for different starches.

In one embodiment the temperature during liquefaction step i) is in the range from 70-100° C., such as between 75-95° C., such as between 75-90° C., preferably between 80-90° C., such as between 82-88° C., such as around 85° C.

In one embodiment, the process of the invention further comprises, prior to the step i), the steps of:
a) reducing the particle size of the starch-containing material, preferably by dry milling;
b) forming a slurry comprising the starch-containing material and water.

The starch-containing starting material, such as whole grains, may be reduced in particle size, e.g., by milling, in order to open up the structure, to increase surface area, and allowing for further processing. Generally, there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry and wet milling are well known in the art of starch processing. According to the present invention dry milling is preferred. In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In another embodiment at least 50%, preferably at least 70%, more preferably at least 80%, especially at least 90% of the starch-containing material fit through a sieve with #6 screen.

The aqueous slurry may contain from 10-55 w/w-% dry solids (DS), preferably 25-45 w/w-% dry solids (DS), more preferably 30-40 w/w-% dry solids (DS) of starch-containing material.

The slurry may be heated to above the initial gelatinization temperature, preferably to between 80-90° C., between pH 4-7, preferably between 4.5-5.0 or 5.0 and 6.0, for 30 minutes to 5 hours, such as around 2 hours.

The alpha-amylase, optional thermostable protease, optional carbohydrate-source generating enzyme, in particular thermostable glucoamylase, may initially be added to the aqueous slurry to initiate liquefaction (thinning). In an embodiment only a portion of the enzymes is added to the aqueous slurry, while the rest of the enzymes are added during liquefaction step i).

Liquefaction step i) is according to the invention carried out for 0.5-5 hours, such as 1-3 hours, such as typically around 2 hours.

The aqueous slurry may in an embodiment be jet-cooked to further gelatinize the slurry before being subjected to liquefaction in step i). The jet-cooking may be carried out at a temperature between 110-145° C., preferably 120-140° C., such as 125-135° C., preferably around 130° C. for about 1-15 minutes, preferably for about 3-10 minutes, especially around about 5 minutes.

### Saccharification and Fermentation

One or more carbohydrate-source generating enzymes, in particular glucoamylase, may be present and/or added during saccharification step ii) and/or fermentation step iii). The carbohydrate-source generating enzyme may preferably be a glucoamylase, but may also be an enzyme selected from the group consisting of: beta-amylase, maltogenic amylase and alpha-glucosidase. The carbohydrate-source generating enzyme added during saccharification step ii) and/or fermentation step iii) is typically different from the optional carbohydrate-source generating enzyme, in particular thermostable glucoamylase, optionally added during liquefaction step i). In a preferred embodiment the carbohydrate-source generating enzymes, in particular glucoamylase, is added together with a fungal alpha-amylase.

Examples of carbohydrate-source generating enzymes, including glucoamylases, can be found in the “Carbohydrate-Source Generating Enzyme Present and/or Added During Saccharification and/or Fermentation”-section below.

One or more alpha-amylases may be present and/or added during saccharification step ii) and/or fermentation step iii). In an embodiment, the alpha-amylase is the Rhizomucor pusillus alpha-amylase with Aspergillus niger glucoamylase linker and SBD disclosed as SEQ ID NO: 11 with the following substitutions: G128D+D143N (activity ratio AGU:AGU:FAU(F): approx. 30:7:1).

One or more trehalases may be present and/or added during saccharification step ii) and/or fermentation step iii). In an embodiment, the trehalase is the Talaromyces funiculosus trehalase disclosed herein as SEQ ID NO: 12, or one having at least 60%, at least 65%, at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 12.

In an embodiment, the trehalase is the Myceliophthora sepedonium trehalase disclosed herein as SEQ ID NO: 28, or one having at least 60%, at least 65%, at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 28.

In an embodiment, the trehalase is part of a blend comprising Gloeophyllum sepiarium glucoamylase disclosed in SEQ ID NO: 13 or one having at least 60%, at least 65%, at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 13, Talaromyces funiculosus trehalase disclosed herein as SEQ ID NO: 12, or one having at least 60%, at least 65%, at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 12, and Rhizomucor pusillus alpha-amylase with Aspergillus niger glucoamylase linker and SBD disclosed as SEQ ID NO: 11 with the following substitutions: G128D+D143N (activity ratio AGU:AGU:FAU(F): approx. 30:7:1) or one having at least 60%, at least 65%, at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 11.

When doing sequential saccharification and fermentation, saccharification step ii) may be carried out at conditions well-known in the art. For instance, the saccharification step ii) may last up to from about 24 to about 72 hours. In an embodiment pre-saccharification is done. Pre-saccharification is typically done for 40-90 minutes at a temperature between 30-65° C., typically about 60° C. Pre-saccharification is in an embodiment followed by saccharification during fermentation in simultaneous saccharification and fermentation (“SSF). Saccharification is typically carried out at temperatures from 20-75° C., preferably from 40-70° C., typically around 60° C., and at a pH between 4 and 5, normally at about pH 4.5.

Simultaneous saccharification and fermentation (“SSF”) is widely used in industrial scale fermentation product production processes, especially ethanol production processes. When doing SSF the saccharification step ii) and the fermentation step iii) are carried out simultaneously. There is no holding stage for the saccharification, meaning that a fermenting organism, such as yeast, and enzyme(s), may be added together. However, it is also contemplated to add the fermenting organism and enzyme(s) separately. SSF is according to the invention typically carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 3.5-5, in particular between 3.8 and 4.3.

### Fermentation Medium

“Fermentation media” or “fermentation medium” refers to the environment in which fermentation is carried out. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “Fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC-North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA. 10567) and NRRL YB-1952 (ARS Culture Collection). Still other S. cerevisiae strains suitable as host cells DBY746, [Alpha] [Eta] 22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The host cell or fermenting organism may be Saccharomyces strain, e.g., Saccharomyces cerevisiae strain produced using the method described and concerned in U.S. Pat. No. 8,257,959-BB. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The strain may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference), strain no. NRRL Y67342 (See, WO2018/098381 incorporated herein by reference), strain nos. NRRL Y67549 and NRRL Y67700 (See, PCT/US2019/018249 incorporated herein by reference), or any strain described in WO2017/087330 (incorporated herein by reference).

The fermenting organisms may be a host cell that expresses a heterologous polypeptide having cellobiohydrolase, endoglucanase, or beta-glucosidase activity (e.g., any polypeptide having cellobiohydrolase, endoglucanase, or beta-glucosidase activity described herein, such as the cellobiohydrolase of SEQ ID NO: 1, 2, 4 or 4; the endoglucanase of SEQ ID NO: 5, or the beta-glucosidase of SEQ ID NO: 6, or a derivative thereof). Any polypeptide having cellobiohydrolase, endoglucanase, or beta-glucosidase activity contemplated for a process, enzyme blend, or composition described herein is also contemplated for expression by a fermenting organism or host cell.

In one embodiment is a recombinant host cell comprising a heterologous polynucleotide encoding a polypeptide having cellobiohydrolase, endoglucanase, or beta-glucosidase (e.g., any polypeptide having cellobiohydrolase, endoglucanase, or beta-glucosidase activity described herein, such as the cellobiohydrolase of SEQ ID NO: 1, 2, 4 or 4; the endoglucanase of SEQ ID NO: 5, or the beta-glucosidase of SEQ ID NO: 6, or a derivative thereof).

In some embodiments, the host cells and/or fermenting organisms comprise one or more heterologous polynucleotides encoding an alpha-amylase, glucoamylase, protease and/or cellulase. Examples of alpha-amylase, glucoamylase, protease and cellulases suitable for expression in the host cells and/or fermenting organisms are described in more detail herein.

The host cells and fermenting organisms described herein may utilize expression vectors comprising the coding sequence of one or more (e.g., two, several) heterologous genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

A construct or vector (or multiple constructs or vectors) comprising the one or more (e.g., two, several) heterologous genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one embodiment, the nucleic acid construct encoding the fusion protein is operably linked to a promoter foreign to the polynucleotide. The promoters may be identical to or share a high degree of sequence identity (e.g., at least about 80%, at least about 85%, at least about 90%, at least about 95%, at least about 96%, at least about 97%, at least about 98%, or at least about 99%) with a selected native promoter.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, xylose reductase (XR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5′-phosphate decarboxylase (URA3) genes. Other suitable promoters may be obtained from S. cerevisiae TDH3, HXT7, PGK1, RPL18B and CCW12 genes. Additional useful promoters for yeast host cells are described by Romanos et al., 1992, Yeast 8:423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, at least about 96%, at least about 97%, at least about 98%, or at least about 99%) with the selected native terminator.

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL10 terminator). Other suitable terminators may be obtained from S. cerevisiae ENO2 or TEF1 genes. Additional useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177:3465-3471).

The control sequence may also be a suitable leader sequence, when transcribed is a non-translated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15:5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used. Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo. Examples of origins of replication for use in a yeast host cell are the 2 micron origin of replication, ARS1, ARS4, the combination of ARS1 and CEN3, and the combination of ARS4 and CEN6.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, Molecular Cloning, A Laboratory Manual, 2d edition, Cold Spring Harbor, New York).

Additional procedures and techniques known in the art for the preparation of recombinant cells for ethanol fermentation, are described in, e.g., WO 2016/045569, the content of which is hereby incorporated by reference.

The host cell or fermenting organism may be in the form of a composition comprising a host cell or fermenting organism (e.g., a yeast strain described herein) and a naturally occurring and/or a non-naturally occurring component.

The host cell or fermenting organism described herein may be in any viable form, including crumbled, dry, including active dry and instant, compressed, cream (liquid) form etc. In one embodiment, the host cell or fermenting organism (e.g., a Saccharomyces cerevisiae yeast strain) is dry yeast, such as active dry yeast or instant yeast. In one embodiment, the host cell or fermenting organism (e.g., a Saccharomyces cerevisiae yeast strain) is crumbled yeast. In one embodiment, the host cell or fermenting organism (e.g., a Saccharomyces cerevisiae yeast strain) is compressed yeast. In one embodiment, the host cell or fermenting organism (e.g., a Saccharomyces cerevisiae yeast strain) is cream yeast.

In one embodiment is a composition comprising a host cell or fermenting organism described herein (e.g., a Saccharomyces cerevisiae yeast strain), and one or more of the component selected from the group consisting of: surfactants, emulsifiers, gums, swelling agent, and antioxidants and other processing aids.

The compositions described herein may comprise a host cell or fermenting organism described herein (e.g., a Saccharomyces cerevisiae yeast strain) and any suitable surfactants. In one embodiment, the surfactant(s) is/are an anionic surfactant, cationic surfactant, and/or nonionic surfactant.

The compositions described herein may comprise a host cell or fermenting organism described herein (e.g., a Saccharomyces cerevisiae yeast strain) and any suitable emulsifier. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a host cell or fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a host cell or fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a host cell or fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a host cell or fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

The host cells and fermenting organisms described herein may also comprise one or more (e.g., two, several) gene disruptions, e.g., to divert sugar metabolism from undesired products to ethanol. In some embodiments, the recombinant host cells produce a greater amount of ethanol compared to the cell without the one or more disruptions when cultivated under identical conditions. In some embodiments, one or more of the disrupted endogenous genes is inactivated.

In certain embodiments, the host cell or fermenting organism provided herein comprises a disruption of one or more endogenous genes encoding enzymes involved in producing alternate fermentative products such as glycerol or other byproducts such as acetate or diols. For example, the cells provided herein may comprise a disruption of one or more of glycerol 3-phosphate dehydrogenase (GPD, catalyzes reaction of dihydroxyacetone phosphate to glycerol 3-phosphate), glycerol 3-phosphatase (GPP, catalyzes conversion of glycerol-3 phosphate to glycerol), glycerol kinase (catalyzes conversion of glycerol 3-phosphate to glycerol), dihydroxyacetone kinase (catalyzes conversion of dihydroxyacetone phosphate to dihydroxyacetone), glycerol dehydrogenase (catalyzes conversion of dihydroxyacetone to glycerol), and aldehyde dehydrogenase (ALD, e.g., converts acetaldehyde to acetate).

Modeling analysis can be used to design gene disruptions that additionally optimize utilization of the pathway. One exemplary computational method for identifying and designing metabolic alterations favoring biosynthesis of a desired product is the OptKnock computational framework, Burgard et al., 2003, Biotechnol. Bioeng. 84:647-657.

The host cells and fermenting organisms comprising a gene disruption may be constructed using methods well known in the art, including those methods described herein. A portion of the gene can be disrupted such as the coding region or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, but are not limited to, a leader, propeptide sequence, signal sequence, transcription terminator, and transcriptional activator.

The host cells and fermenting organisms comprising a gene disruption may be constructed by gene deletion techniques to eliminate or reduce expression of the gene. Gene deletion techniques enable the partial or complete removal of the gene thereby eliminating their expression. In such methods, deletion of the gene is accomplished by homologous recombination using a plasmid that has been constructed to contiguously contain the 5′ and 3′ regions flanking the gene.

The host cells and fermenting organisms comprising a gene disruption may also be constructed by introducing, substituting, and/or removing one or more (e.g., two, several) nucleotides in the gene or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, 1985, Science 229:4719; Lo et al., 1985, Proc. Natl. Acad. Sci. U.S.A. 81:2285; Higuchi et al., 1988, Nucleic Acids Res 16:7351; Shimada, 1996, Meth. Mol. Biol. 57:157; Ho et al., 1989, Gene 77:61; Horton et al., 1989, Gene 77:61; and Sarkar and Sommer, 1990, BioTechniques 8:404.

The host cells and fermenting organisms comprising a gene disruption may also be constructed by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The host cells and fermenting organisms comprising a gene disruption may also be constructed by the process of gene conversion (see, for example, Iglesias and Trautner, 1983, Molecular General Genetics 189:73-76). For example, in the gene conversion method, a nucleotide sequence corresponding to the gene is mutagenized in vitro to produce a defective nucleotide sequence, which is then transformed into the recombinant strain to produce a defective gene. By homologous recombination, the defective nucleotide sequence replaces the endogenous gene. It may be desirable that the defective nucleotide sequence also comprises a marker for selection of transformants containing the defective gene.

The host cells and fermenting organisms comprising a gene disruption may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the gene may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent strain to be mutagenized in the presence of the mutagenizing agent of choice under suitable conditions, and selecting for mutants exhibiting reduced or no expression of the gene.

A nucleotide sequence homologous or complementary to a gene described herein may be used from other microbial sources to disrupt the corresponding gene in a recombinant strain of choice.

In one embodiment, the modification of a gene in the recombinant cell is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived there from, or cereals. Contemplated are also waxy and non-waxy types of corn and barley. In a preferred embodiment the starch-containing material, used for ethanol production according to the invention, is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery

Subsequent to fermentation, or SSF, the fermentation product may be separated from the fermentation medium. The slurry may be distilled to extract the desired fermentation product (e.g., ethanol). Alternatively, the desired fermentation product may be extracted from the fermentation medium by micro or membrane filtration techniques. The fermentation product may also be recovered by stripping or other method well known in the art.

Alpha-Amylase Present and/or Added During Liquefaction

According to the invention an alpha-amylase is present and/or added during liquefaction together with an optional thermostable protease, optional carbohydrate-source generating enzyme, in particular a thermostable glucoamylase, and/or optional pullulanase.

The alpha-amylase added during liquefaction step i) may be any alpha-amylase. Preferred are bacterial alpha-amylases, which typically are stable at temperature used during liquefaction.

Any alpha-amylase herein contemplated as being present and/or added during liquefaction is also contemplated for expression by a fermenting organism or host cell.

The term “bacterial alpha-amylase” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase of SEQ ID NO: 3 in WO 99/19467, the Bacillus amyloliquefaciens alpha-amylase of SEQ ID NO: 5 in WO 99/19467, and the Bacillus licheniformis alpha-amylase of SEQ ID NO: 4 in WO 99/19467 (all sequences are hereby incorporated by reference). In an embodiment the alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467.

In an embodiment the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO 99/19467 or SEQ ID NO: 14 herein.

In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated so it has around 491 amino acids compared to SEQ ID NO: 3 in WO 99/19467.

The Bacillus alpha-amylase may also be a variant and/or hybrid. Examples of such a variant can be found in any of WO 96/23873, WO 96/23874, WO 97/41213, WO 99/19467, WO 00/60059, and WO 02/10355 (all documents are hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, I181 and/or G182, preferably a double deletion disclosed in WO 96/23873-see, e.g., page 20, lines 1-10 (hereby incorporated by reference), preferably corresponding to deletion of positions I181 and G182 compared to the amino acid sequence of Bacillus stearothermophilus alpha-amylase set forth in SEQ ID NO: 3 disclosed in WO 99/19467 or SEQ ID NO: 14 herein or the deletion of amino acids R179 and G180 using SEQ ID NO: 3 in WO 99/19467 or SEQ ID NO: 14 herein for numbering (which reference is hereby incorporated by reference). Even more preferred are Bacillus alpha-amylases, especially Bacillus stearothermophilus alpha-amylases, which have a double deletion corresponding to a deletion of positions 181 and 182 and further comprise a N193F substitution (also denoted I181*+G182*+N193F) compared to the wild-type BSG alpha-amylase amino acid sequence set forth in SEQ ID NO: 3 disclosed in WO 99/19467 or SEQ ID NO: 14 herein. The bacterial alpha-amylase may also have a substitution in a position corresponding to S239 in the Bacillus licheniformis alpha-amylase shown in SEQ ID NO: 4 in WO 99/19467, or a S242 variant of the Bacillus stearothermophilus alpha-amylase of SEQ ID NO: 3 in WO 99/19467 or SEQ ID NO: 14 herein.

In an embodiment the variant is a S242A, E or Q variant, preferably a S242Q variant, of the Bacillus stearothermophilus alpha-amylase (using SEQ ID NO: 14 herein for numbering).

In an embodiment the variant is a position E188 variant, preferably E188P variant of the Bacillus stearothermophilus alpha-amylase (using SEQ ID NO: 14 herein for numbering).

The bacterial alpha-amylase may in an embodiment be a truncated alpha-amylase. Especially the truncation is so that the Bacillus stearothermophilus alpha-amylase shown in SEQ ID NO: 3 in WO 99/19467 or SEQ ID NO: 14 herein, is around 491 amino acids long, such as from 480 to 495 amino acids long.

Most importantly, a suitable alpha-amylase for use in liquefaction must have sufficient therm-stability, and thus accordingly any alpha-amylase having a T½ (min) at pH 4.5, 85° C., 0.12 mM CaCl₂)) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70, may be used.

According to the invention the alpha-amylase may be a thermostable alpha-amylase, such as a thermostable bacterial alpha-amylase, preferably from Bacillus stearothermophilus. In an embodiment the alpha-amylase used according to the invention has a T½ (min) at pH 4.5, 85° C., 0.12 mM CaCl₂) of at least 10.

In an embodiment the thermostable alpha-amylase has a T½ (min) at pH 4.5, 85° C., 0.12 mM CaCl₂), of at least 15.

In an embodiment the thermostable alpha-amylase has a T½ (min) at pH 4.5, 85° C., 0.12 mM CaCl₂), of as at least 20.

In an embodiment the thermostable alpha-amylase has a T½ (min) at pH 4.5, 85° C., 0.12 mM CaCl₂), of as at least 25.

In an embodiment the thermostable alpha-amylase has a T½ (min) at pH 4.5, 85° C., 0.12 mM CaCl₂), of as at least 30.

In an embodiment the thermostable alpha-amylase has a T½ (min) at pH 4.5, 85° C., 0.12 mM CaCl₂), of as at least 40.

In an embodiment the thermostable alpha-amylase has a T½ (min) at pH 4.5, 85° C., 0.12 mM CaCl₂), of at least 50.

In an embodiment the thermostable alpha-amylase has a T½ (min) at pH 4.5, 85° C., 0.12 mM CaCl₂), of at least 60.

In an embodiment the thermostable alpha-amylase has a T½ (min) at pH 4.5, 85° C., 0.12 mM CaCl₂), between 10-70.

In an embodiment the thermostable alpha-amylase has a T½ (min) at pH 4.5, 85° C., 0.12 mM CaCl₂), between 15-70.

In an embodiment the thermostable alpha-amylase has a T½ (min) at pH 4.5, 85° C., 0.12 mM CaCl₂), between 20-70.

In an embodiment the thermostable alpha-amylase has a T½ (min) at pH 4.5, 85° C., 0.12 mM CaCl₂), between 25-70.

In an embodiment the thermostable alpha-amylase has a T½ (min) at pH 4.5, 85° C., 0.12 mM CaCl₂), between 30-70.

In an embodiment the thermostable alpha-amylase has a T½ (min) at pH 4.5, 85° C., 0.12 mM CaCl₂), between 40-70.

In an embodiment the thermostable alpha-amylase has a T½ (min) at pH 4.5, 85° C., 0.12 mM CaCl₂), between 50-70.

In an embodiment the thermostable alpha-amylase has a T½ (min) at pH 4.5, 85° C., 0.12 mM CaCl₂), between 60-70.

In an embodiment of the invention the alpha-amylase is an bacterial alpha-amylase, preferably derived from the genus Bacillus, especially a strain of Bacillus stearothermophilus, in particular the Bacillus stearothermophilus as disclosed in WO 99/019467 as SEQ ID NO: 3 (SEQ ID NO: 14 herein) with one or two amino acids deleted at positions R179, G180, I181 and/or G182, in particular with R179 and G180 deleted, or with I181 and G182 deleted, with mutations in below list of mutations.

In preferred embodiments the Bacillus stearothermophilus alpha-amylases have double deletion I181+G182, and optional substitution N193F, further comprising mutations selected from below list.

In a preferred embodiment the alpha-amylase is selected from the following group of Bacillus stearothermophilus alpha-amylase variants (using SEQ ID NO: 14 for numbering):
I181*+G182*+N193F+E129V+K177L+R179E;
I181*+G182*+N193F+V59A+Q89R+E129V+K177L+R179E+H208Y+K220P+N224L+Q254S
I181*+G182*+N193F+V59A+Q89R+E129V+K177L+R179E+Q254S+M284V;
I181*+G182*+N193F+V59A+E129V+K177L+R179E+Q254S+M284V;
I181*+G182*+N193F+E129V+K177L+R179E+K220P+N224L+S242Q+Q254S;
I181*+G182*+V59A+E129V+K177L+R179E+Q254S+M284V+V212T+Y268G+N293Y+T297N;
I181*+G182*+V59A+E129V+K177L+R179E+Q254S+M284V+V212T+Y268G+N293Y+T297N+S173N+E188P+H208Y+S242Y+K2791;
I181*+G182*+V59A+E129V+K177L+R179S+Q254S+M284V+V212T+Y268G+N293Y+T297N+A184Q+E188P+T191N
I181*+G182*+V59A+E129V+K177L+R179S+Q254S+M284V+V212T+Y268G+N293Y+T297N+A184Q+E188P+T191N+S242Y+K2791;
I181*+G182*+V59A+E129V+K177L+R179E+Q254S+M284V+V212T+Y268G+N293Y+T297N+E188P+K279W;
I181*+G182*+V59A+E129V+K177L+R179E+Q254S+M284V+V212T+Y268G+N293Y+T297N+W115D+D117Q+T133P; and
   wherein the variant has at least 85%, at least 90%, at least 95%, at least 96%, at least 97%, at least 98%, or at least 99% sequence identity to SEQ ID NO: 14.

It should be understood, that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 14 herein, or variants thereof, are truncated in the C-terminal and are typically around 491 amino acids long, such as from 480-495 amino acids long.

In a preferred embodiment the alpha-amylase variant may be an enzyme having a degree of identity of at least 60%, e.g., at least 70%, at least 80%, at least 90%, at least 95%, at least 91%, at least 92%, at least 93%, at least 94%, at least 95%, at least 96%, at least 97%, at least 98% or at least 99%, but less than 100% to the sequence shown in SEQ ID NO: 3 in WO 99/19467 or SEQ ID NO: 14 herein.

Protease Present and/or Added During Liquefaction

According to the invention a thermostable protease is optionally present and/or added during liquefaction together with an alpha-amylase, and optionally a carbohydrate-source generating enzyme, in particular a thermostable glucoamylase, and/or optionally a pullulanase.

Any protease herein contemplated as being present and/or added during liquefaction is also contemplated for expression by a fermenting organism or host cell.

Proteases are classified on the basis of their catalytic mechanism into the following groups: Serine proteases(S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section, of which the so-called “AZCL-Casein Assay” is the preferred assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu determined by the AZCL-casein assay described in the “Materials & Methods” section.

There are no limitations on the origin of the protease used in a process of the invention as long as it fulfills the thermostability properties defined below.

In one embodiment the protease is of fungal origin.

The protease may be a variant of, e.g., a wild-type protease as long as the protease has the thermostability properties defined herein.

In a particular embodiment the thermostable protease is a variant of a metallo protease as defined above. In an embodiment the thermostable protease used in a process of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of the mature part of the metallo protease shown in SEQ ID NO: 2 disclosed in WO 2003/048353 or the mature part of SEQ ID NO: 1 in WO 2010/008841 and shown as SEQ ID NO: 15 herein further with mutations selected from below list:
S5*+D79L+S87P+A112P+D142L;
D79L+S87P+A112P+T124V+D142L;
S5*+N26R+D79L+S87P+A112P+D142L;
N26R+T46R+D79L+S87P+A112P+D142L;
T46R+D79L+S87P+T116V+D142L;
D79L+P81R+S87P+A112P+D142L;
A27K+D79L+S87P+A112P+T124V+D142L;
D79L+Y82F+S87P+A112P+T124V+D142L;
D79L+Y82F+S87P+A112P+T124V+D142L;
D79L+S87P+A112P+T124V+A126V+D142L;
D79L+S87P+A112P+D142L;
D79L+Y82F+S87P+A112P+D142L;
S38T+D79L+S87P+A112P+A126V+D142L;
D79L+Y82F+S87P+A112P+A126V+D142L;
A27K+D79L+S87P+A112P+A126V+D142L;
D79L+S87P+N98C+A112P+G135C+D142L;
D79L+S87P+A112P+D142L+T141C+M161C;
S36P+D79L+S87P+A112P+D142L;
A37P+D79L+S87P+A112P+D142L;
S49P+D79L+S87P+A112P+D142L;
S50P+D79L+S87P+A112P+D142L;
D79L+S87P+D104P+A112P+D142L;
D79L+Y82F+S87G+A112P+D142L;
S70V+D79L+Y82F+S87G+Y97W+A112P+D142L;
D79L+Y82F+S87G+Y97W+D104P+A112P+D142L;
S70V+D79L+Y82F+S87G+A112P+D142L;
D79L+Y82F+S87G+D104P+A112P+D142L;
D79L+Y82F+S87G+A112P+A126V+D142L;
Y82F+S87G+S70V+D79L+D104P+A112P+D142L;
Y82F+S87G+D79L+D104P+A112P+A126V+D142L;
A27K+D79L+Y82F+S87G+D104P+A112P+A126V+D142L;
A27K+Y82F+S87G+D104P+A112P+A126V+D142L;
A27K+D79L+Y82F+D104P+A112P+A126V+D142L;
A27K+Y82F+D104P+A112P+A126V+D142L;
A27K+D79L+S87P+A112P+D142L;
D79L+S87P+D142L.

In a preferred embodiment the thermostable protease is a variant of the metallo protease disclosed as the mature part of SEQ ID NO: 2 disclosed in WO 2003/048353 or the mature part of SEQ ID NO: 1 in WO 2010/008841 or SEQ ID NO: 15 herein with the following mutations:
D79L+S87P+A112P+D142L;
D79L+S87P+D142L; or
A27K+D79L+Y82F+S87G+D104P+A112P+A126V+D142L.

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 15 herein.

The thermostable protease may also be derived from any bacterium as long as the protease has the thermostability properties defined according to the invention. In one embodiment the protease is a serine protease, particularly a S8 protease. Preferred proteases are, serine proteases, particularly an S8 serine protease derived from a strain of Pyrococcus, preferably a strain of Pyrococcus furiosus, or derived from a strain of Thermococcus, preferably Themococcus thioreducens, or derived from a strain of Palaeococcus, preferably Palaeococcus ferrophilus.

In an embodiment the thermostable protease is derived from a strain of the bacterium Pyrococcus, such as a strain of Pyrococcus furiosus (pfu protease).

In an embodiment the protease is one shown as SEQ ID NO: 1 in U.S. Pat. No. 6,358,726-B1 (Takara Shuzo Company), SEQ ID NO: 16 herein.

In another embodiment the thermostable protease is one disclosed in SEQ ID NO: 16 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 16 herein.

The Pyrococcus furiosus protease is a thermostable protease according to the invention. The Pyrococcus furiosus protease (PfuS) was found to have a thermostability of 110% (80° C./70° C.) and 103% (90° C./70° C.) at pH 4.5 determined as described in Example 2 herein.

In an embodiment the thermostable protease is derived from a strain of the bacterium Palaeococcus, such as a strain of Palaeococcus ferrophilus. In an embodiment the protease is the one shown as SEQ ID NO: 17 herein. In another embodiment the thermostable protease is one disclosed in SEQ ID NO: 17 herein or a protease having at least 80% identity, such as at least 85%, such as at least 90%, such as at least 95%, such as at least 96%, such as at least 97%, such as at least 98%, such as at least 99% identity to SEQ ID NO: 17.

In one embodiment a thermostable protease used in a process of the invention has a thermostability value of more than 20% determined as Relative Activity at 80° C./70° C.

In an embodiment the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In an embodiment protease has a thermostability of between 20 and 50%, such as between 20 and 40%, such as 20 and 30% determined as Relative Activity at 80° C./70° C.

In an embodiment the protease has a thermostability between 50 and 115%, such as between 50 and 70%, such as between 50 and 60%, such as between 100 and 120%, such as between 105 and 115% determined as Relative Activity at 80° C./70° C.

In an embodiment the protease has a thermostability value of more than 10% determined as Relative Activity at 85° C./70° C.

In an embodiment the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In an embodiment the protease has a thermostability of between 10 and 50%, such as between 10 and 30%, such as between 10 and 25% determined as Relative Activity at 85° C./70° C.

In an embodiment the protease has more than 20%, more than 30%, more than 40%, more than 50%, more than 60%, more than 70%, more than 80%, more than 90% determined as Remaining Activity at 80° C.; and/or In an embodiment the protease has more than 20%, more than 30%, more than 40%, more than 50%, more than 60%, more than 70%, more than 80%, more than 90% determined as Remaining Activity at 84° C.

In an embodiment the protease may have a themostability for above 90, such as above 100 at 85° C. as determined using the Zein-BCA assay.

In an embodiment the protease has a themostability above 60%, such as above 90%, such as above 100%, such as above 110% at 85° C. as determined using the Zein-BCA assay.

In an embodiment protease has a themostability between 60-120, such as between 70-120%, such as between 80-120%, such as between 90-120%, such as between 100-120%, such as 110-120% at 85° C. as determined using the Zein-BCA assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by the AZCL-casein assay.

In an embodiment the protease is derived from a strain of Thermobifida, such as the Thermobifida cellulosytica protease shown in SEQ ID NO: 18 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 18.

In an embodiment the protease is derived from a strain of Thermobifida, such as the Thermobifida fusca protease shown in SEQ ID NO: 19 herein (referred to as SEQ ID NO: 8 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 19.

In an embodiment the protease is derived from a strain of Thermobifida, such as the Thermobifida halotolerans protease shown in SEQ ID NO: 20 herein (referred to as SEQ ID NO: 10 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 20.

In an embodiment the protease is derived from a strain of Thermococcus, such as the Thermococcus nautili protease shown in SEQ ID NO: 21 herein (referred to as SEQ ID NO: 3 in WO2018/169780A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 21.

Carbohydrate-Source Generating Enzyme Present and/or Added During Liquefaction

According to the invention a carbohydrate-source generating enzyme, in particular a glucoamylase, preferably a thermostable glucoamylase, may optionally be present and/or added during liquefaction together with an alpha-amylase and an optional thermostable protease. As mentioned above, a pullulanase may also be optionally be present and/or added during liquefaction step i).

Any carbohydrate-source generating enzymes (e.g., glucoamylase) herein contemplated as being present and/or added during liquefaction is also contemplated for expression by a fermenting organism or host cell.

The term “carbohydrate-source generating enzyme” includes any enzymes generating fermentable sugars. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrates may be converted directly or indirectly to the desired fermentation product, preferably ethanol. According to the invention a mixture of carbohydrate-source generating enzymes may be used. Specific examples include glucoamylase (being glucose generators), beta-amylase and maltogenic amylase (being maltose generators).

In a preferred embodiment the carbohydrate-source generating enzyme is thermostable. The carbohydrate-source generating enzyme, in particular thermostable glucoamylase, may be added together with or separately from the alpha-amylase and the thermostable protease.

In a specific and preferred embodiment the carbohydrate-source generating enzyme is a thermostable glucoamylase, preferably of fungal origin, preferably a filamentous fungi, such as from a strain of the genus Penicillium, especially a strain of Penicillium oxalicum, in particular the Penicillium oxalicum glucoamylase disclosed as SEQ ID NO: 2 in PCT/CN10/071753 published as WO 2011/127802 (which is hereby incorporated by reference) and shown in SEQ ID NO: 22 herein.

In an embodiment the thermostable glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO2011/127802 or SEQ ID NOs: 23 herein.

In a preferred embodiment the carbohydrate-source generating enzyme is a variant of the Penicillium oxalicum glucoamylase disclosed as SEQ ID NO: 2 in WO2011/127802 and SEQ ID NO: 22 herein, having a K79V substitution (using the mature sequence shown in SEQ ID NO: 22 for numbering). The K79V glucoamylase variant has reduced sensitivity to protease degradation relative to the parent as disclosed in co-pending U.S. application No. 61/531,189 or PCT/US12/053779 (which are hereby incorporated by reference).

In an embodiment the carbohydrate-source generating enzyme, in particular thermostable glucoamylase, is derived from Penicillium oxalicum.

In an embodiment the thermostable glucoamylase is a variant of the Penicillium oxalicum glucoamylase disclosed as SEQ ID NO: 2 in WO2011/127802 and shown in SEQ ID NO: 22 herein. In a preferred embodiment the Penicillium oxalicum glucoamylase is the one disclosed as SEQ ID NO: 2 in WO2011/127802 and shown in SEQ ID NO: 22 herein having Val (V) in position 79 (using SEQ ID NO: 22 for numbering).

In an embodiment these variants have reduced sensitivity to protease degradation.

In an embodiment these variants have improved thermostability compared to the parent.

More specifically, in an embodiment the glucoamylase has a K79V substitution (using SEQ ID NO: 22 for numbering), corresponding to the PE001 variant, and further comprises at least one of the following substitutions or combination of substitutions:
P11F+T65A+Q327F; or
P2N+P4S+P11F+T65A+Q327F; or
P11F+D26C+K33C+T65A+Q327F; or
P2N+P4S+P11F+T65A+Q327W+E501V+Y504T; or
P2N+P4S+P11F+T65A+Q327F+E501V+Y504T; or
P11F+T65A+Q327W+E501V+Y504T.

The carbohydrate-source generating enzyme, in particular, may be added in amounts from 0.1-100 micrograms EP/g, such as 0.5-50 micrograms EP/g, such as 1-25 micrograms EP/g, such as 2-12 micrograms EP/g DS.

Carbohydrate-Source Generating Enzyme Present and/or Added During Saccharification and/or Fermentation

According to the invention a carbohydrate-source generating enzyme, preferably a glucoamylase, may be present and/or added during saccharification and/or fermentation.

In a preferred embodiment the carbohydrate-source generating enzyme is a glucoamylase, of fungal origin, preferably from a stain of Aspergillus, preferably A. niger, A. awamori, or A. oryzae; or a strain of Trichoderma, preferably T. reesei; or a strain of Talaromyces, preferably T. emersonii, or or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

Any glucoamylase contemplated as being present and/or added during saccharification and/or fermentation is also contemplated for expression by a fermenting organism or host cell.

### Glucoamylases

According to the invention the glucoamylase present and/or added during saccharification and/or fermentation may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). In a preferred embodiment the glucoamylase used during saccharification and/or fermentation is the Talaromyces emersonii glucoamylase disclosed in WO 99/28448.

Contemplated fungal glucoamylases include particularly glucoamylases derived from Talaromyces, preferably T. emersonii, or or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

In one embodiment the glucoamylase is derived from a strain of the genus Trametes, in particular a strain of Trametes cingulata, disclosed in WO 2006/069289 or in SEQ ID NO: 24 herein. In one embodiment the glucoamylase is derived from a strain of the genus Talaromyces, in particular a strain of Talaromyces emersonii disclosed in SEQ ID NO: 27 herein.

In another embodiment the glucoamylase is derived from a strain of the genus Pycnoporus, in particular a strain of Pycnoporus sanguineus as described in WO 2011/066576 (SEQ ID NOs 2, 4 or 6) or SEQ ID NO: 25 herein, or from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is SEQ ID NO: 13 herein. In another embodiment the glucoamylase is SEQ ID NO: 26 herein. In an embodiment the glucoamylase is derived from a strain of the genus Nigrofomes, in particular a strain of Nigrofomes sp. disclosed in WO 2012/064351 as SEQ ID NO: 2. Contemplated are also glucoamylases which exhibit a high identity to any of the above mentioned glucoamylases, i.e., at least 70%, at least 75%, at least 80%, at least 85%, at least 90%, at least 95%, at least 96%, at least 97%, at least 98%, at least 99% or even 100% identity to any one of the mature parts of the enzyme sequences mentioned above, such as any of SEQ ID NOS: 24, 25, 26, 27, or 28 herein.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

In an embodiment the glucoamylase is added as a blend further comprising an alpha-amylase. In a preferred embodiment the alpha-amylase is a fungal alpha-amylase, especially an acid fungal alpha-amylase. The alpha-amylase is typically a side activity.

In an embodiment the glucoamylase is a blend comprising Talaromyces emersonii glucoamylase disclosed in WO 99/28448 as SEQ ID NO: 7 or SEQ ID NO: 27 herein and Trametes cingulata glucoamylase disclosed in WO 06/069289 and SEQ ID NO: 24 herein.

In an embodiment the glucoamylase is a blend comprising Talaromyces emersonii glucoamylase disclosed SEQ ID NO: 27, Trametes cingulata glucoamylase disclosed as SEQ ID NO: 24, and Rhizomucor pusillus alpha-amylase with Aspergillus niger glucoamylase linker and SBD disclosed as V039 in Table 5 in WO 2006/069290 and as SEQ ID NO: 11 herein, preferably with the following substitutions: G128D+D143N.

In an embodiment the glucoamylase is a blend comprising Gloeophyllum sepiarium glucoamylase shown as SEQ ID NO: 2 in WO 2011/068803 (SEQ ID NO: 13 herein) and Rhizomucor pusillus with an Aspergillus niger glucoamylase linker and starch-binding domain (SBD), disclosed SEQ ID NO: 3 in WO 2013/006756 (SEQ ID NO: 11 herein) with the following substitutions: G128D+D143N.

In an embodiment the Rhizomucor pusillus alpha-amylase or the Rhizomucor pusillus alpha-amylase with an Aspergillus niger glucoamylase linker and starch-binding domain (SBD) has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R+V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 3 in WO 2013/006756 for numbering).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE and AMG™ E (from Novozymes A/S); OPTIDEX™ 300, GC480, GC417 (from DuPont-Genencor); AMIGASE™ and AMIGASE™ PLUS (from DSM); G-ZYME™ G900, G-ZYME™ and G990 ZR (from DuPont-Genencor).

Cellulolytic Composition Present and/or Added During Saccharification and/or Fermentation

According to the invention a cellulolytic composition is present during fermentation or simultaneous saccharification and fermentation (SSF).

The cellulolytic composition may be any cellulolytic composition, comprising a beta-glucosidase, a cellobiohydrolase and an endoglucanase.

Any cellulase described herein contemplated as being present and/or added during saccharification and/or fermentation is also contemplated for expression by a fermenting organism or host cell.

The compositions may comprise a polypeptide of the present invention as the major enzymatic component, e.g., a mono-component composition. Alternatively, the compositions may comprise multiple enzymatic activities, such as one or more (e.g., several) additional enzymes selected from the group consisting of a cellulase, a polypeptide having cellulolytic enhancing activity, a hemicellulase, an esterase, an expansin, a laccase, a ligninolytic enzyme, a pectinase, a peroxidase, a protease, and a swollenin.

The compositions may be prepared in accordance with methods known in the art and may be in the form of a liquid or a dry composition. The compositions may be stabilized in accordance with methods known in the art.

The compositions may be a fermentation broth formulation or a cell composition, as described herein. Consequently, the present invention also relates to fermentation broth formulations and cell compositions comprising a polypeptide having cellobiohydrolase, endoglucanase, or beta-glucosidase activity of the present invention. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compostions may further comprise a preservative and/or anti-microbial (e.g., bacteriostatic) agent, including, but not limited to, sorbitol, sodium chloride, potassium sorbate, and others known in the art.

In some embodiments, the cell-killed whole broth or composition includes cellulolytic enzymes including, but not limited to, (i) endoglucanases (EG) or 1,4-D-glucan-4-glucanohydrolases (EC 3.2.1.4), (ii) exoglucanases, including 1,4-D-glucan glucanohydrolases (also known as cellodextnnases) (EC 3.2.1.74) and 1,4-D-glucan cellobiohydrolases (exo-cellobiohydrolases, CBH) (EC 3.2.1.91), and (iii) beta-glucosidase (BG) or beta-glucoside glucohydrolases (EC 3.2.1.21).

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

Additional examples of suitable cellulolytic composition can be found in WO 2008/151079 and co-pending patent application PCT/US12/052163 published as WO2013/028928 which are incorporated by reference.

In preferred embodiments the cellulolytic composition is derived from a strain of Trichoderma, Humicola, or Chrysosporium.

In an embodiment the cellulolytic composition is derived from a strain of Trichoderma reesei, Humicola insolens and/or Chrysosporium lucknowense.

In an embodiment the cellulolytic composition comprises a beta-glucosidase, preferably one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigatus, such as one disclosed in WO2005/047499 or SEQ ID NO: 8 herein or an Aspergillus fumigatus beta-glucosidase variant disclosed in WO 2012/044915 (Novozymes), such as one with the following substitutions F100D, S283G, N456E, F512Y; or a strain of the genus a strain Penicillium, such as a strain of the Penicillium brasilianum disclosed in WO 2007/019442, or a strain of the genus Trichoderma, such as a strain of Trichoderma reesei.

In an embodiment the cellulolytic composition comprises a GH61 polypeptide having cellulolytic enhancing activity such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigatus, such as the one described in WO 2010/138754 as SEQ ID NO: 1 and SEQ ID NO: 2; or one derived from a strain derived from Penicillium, such as a strain of Penicillium emersonii, such as the one disclosed in WO 2011/041397 or SEQ ID NO: 7 herein.

In an embodiment the cellulolytic composition comprises a cellobiohydrolase I (CBH I), such as one derived from a strain of the genus Aspergillus, such as a strain of Aspergillus fumigatus, such as the Cel7a CBHI disclosed in SEQ ID NO: 2 in WO 2011/057140 or SEQ ID NO: 9 herein, or a strain of the genus Trichoderma, such as a strain of Trichoderma reesei.

In an embodiment the cellulolytic composition comprises a cellobiohydrolase II (CBH II, such as one derived from a strain of the genus Aspergillus, such as a strain of Aspergillus fumigatus or SEQ ID NO: 10 herein; or a strain of the genus Trichoderma, such as Trichoderma reesei, or a strain of the genus Thielavia, such as a strain of Thielavia terrestris, such as cellobiohydrolase II CEL6A from Thielavia terrestris.

In an embodiment the cellulolytic composition comprises a GH61 polypeptide having cellulolytic enhancing activity and a beta-glucosidase.

In an embodiment the cellulolytic composition comprises a GH61 polypeptide having cellulolytic enhancing activity, a beta-glucosidase, and a CBH I.

In an embodiment the cellulolytic composition comprises a GH61 polypeptide having cellulolytic enhancing activity, a beta-glucosidase, a CBH I, and a CBH II.

In an embodiment the cellulolytic composition is a Trichoderma reesei cellulolytic enzyme composition, further comprising Thermoascus aurantiacus GH61A polypeptide having cellulolytic enhancing activity (SEQ ID NO: 2 in WO 2005/074656), and Aspergillus oryzae beta-glucosidase fusion protein (WO 2008/057637).

In an embodiment the cellulolytic composition is a Trichoderma reesei cellulolytic enzyme composition, further comprising Thermoascus aurantiacus GH61A polypeptide having cellulolytic enhancing activity (SEQ ID NO: 2 in WO 2005/074656) and Aspergillus fumigatus beta-glucosidase (SEQ ID NO: 2 of WO 2005/047499) or SEQ ID NO: 8 herein.

In an embodiment the cellulolytic composition is a Trichoderma reesei cellulolytic enzyme composition further comprising Penicillium emersonii GH61A polypeptide having cellulolytic enhancing activity disclosed in WO 2011/041397 and Aspergillus fumigatus beta-glucosidase (SEQ ID NO: 2 of WO 2005/047499) or SEQ ID NO: 8 herein or a variant thereof with the following substitutions F100D, S283G, N456E, F512Y.

In an embodiment, the cellulolytic composition, for example a Trichoderma reesei cellulolytic enzyme composition, comprises one or more polypeptides selected from the group consisting of:
beta-glucosidase;
cellobiohydrolase I; and
endoglucanase I, or a mixture of two or three thereof.

In an embodiment, the cellulolytic composition, for example a Trichoderma reesei cellulolytic enzyme composition, comprises one or more of the following components:
(i) an Aspergillus fumigatus beta-glucosidase or a variant thereof;
(ii) an Aspergillus fumigatus cellobiohydrolase I; and
(iii) a Trichoderma reesei endoglucanase I.

In an embodiment, the cellulolytic composition is a Trichoderma reesei cellulolytic composition further comprising:
(i) an Aspergillus fumigatus beta-glucosidase disclosed in SEQ ID NO: 8 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 8; (ii) a cellobiohydrolase I (CBH I), such as one derived from a strain of the genus Aspergillus, such as a strain of Aspergillus fumigatus, such as the CBHI disclosed as SEQ ID NO: 9, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 9; and (iii) an endoglucanase I (EGI), such as one derived from a strain of the genus Trichoderma, such as a strain of Trichoderma reesei, such as the EGI disclosed as SEQ ID NO: 29, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 29.

In a preferred embodiment the cellulolytic composition comprising one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

In an preferred embodiment the cellulolytic composition is derived from Trichoderma reesei comprising GH61A polypeptide having cellulolytic enhancing activity derived from a strain of Penicillium emersonii (SEQ ID NO: 2 in WO 2011/041397 or SEQ ID NO: 7 herein), Aspergillus fumigatus beta-glucosidase (SEQ ID NO: 2 in WO 2005/047499 SEQ ID NO: 8 herein) variant F100D, S283G, N456E, F512Y) disclosed in WO 2012/044915; Aspergillus fumigatus Cel7A CBH1 disclosed as SEQ ID NO: 6 in WO2011/057140 (SEQ ID NO: 9 herein) and Aspergillus fumigatus CBH II disclosed as SEQ ID NO: 18 in WO 2011/057140 (SEQ ID NO: 10 herein).

In an embodiment the cellulolytic composition is dosed from 0.0001-3 mg EP/g DS, preferably, 0.0005-2 mg EP/g DS, preferably 0.001-1 mg/g DS, more preferably 0.005-0.5 mg EP/g DS, and even more preferably 0.01-0.1 mg EP/g DS.

Hemicellulolytic Composition Present and/or Added During Saccharification and/or Fermentation

According to the invention a hemicellulolytic composition is present during fermentation or simultaneous saccharification and fermentation (SSF).

The hemicellulolytic composition may be any hemicellulolytic composition, comprising a xylanase, and an arabinofuranosidase.

Any hemicellulase described herein contemplated as being present and/or added during saccharification and/or fermentation is also contemplated for expression by a fermenting organism or host cell.

The compositions may be prepared in accordance with methods known in the art and may be in the form of a liquid or a dry composition. The compositions may be stabilized in accordance with methods known in the art.

The compositions may be a fermentation broth formulation or a cell composition, as described herein.

In an embodiment, the hemicellulolytic composition comprises:
(i) a GH10 xylanase; and
(ii) a GH62 arabinofuranosidase.

In an embodiment, the hemicellulolytic composition comprises:
(i) a GH10 xylanase from Talaromyces leycettanus; and
(ii) a GH62 arabinofuranosidase from Talaromyces pinophilus.

In an embodiment, the hemicellulolytic composition comprises:
(i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and
(ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

In an embodiment the hemicellulolytic composition is dosed from 0.0001-3 mg EP/g DS, preferably, 0.0005-2 mg EP/g DS, preferably 0.001-1 mg/g DS, more preferably 0.005-0.5 mg EP/g DS, and even more preferably 0.01-0.1 mg EP/g DS.

### Fiber Degrading Enzyme Blend

Aspects of the present invention relate to a fiber degrading enzyme blend comprising at least one polypeptide of the present invention (e.g., a cellulolytic composition) and a hemicellulolytic composition.

Any cellulase/cellulolytic composition and hemicellulase/hemicellulolytic composition described herein can be used in the fiber degrading enzyme blend.

In an example embodiment, the fiber degrading enzyme blend comprises at least one polypeptide having cellobiohydrolase activity, endoglucanase activity, or beta-glucosidase activity, and a hemicellulolytic composition comprising a xylanase and an arabinofuranosidase, wherein the at least one polypeptide having cellobiohydrolase activity, endoglucanase activity, or beta-glucosidase activity comprises at least one, at least two, at least three, at least four, at least five polypeptides, or at least six polypeptides selected from the group consisting of: (i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1; (ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2; (iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3; (iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4; (v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and (vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

In an example embodiment, the fiber degrading enzyme blend comprises at least one polypeptide having cellobiohydrolase activity, endoglucanase activity, or beta-glucosidase activity, and a hemicellulolytic composition comprising a GH10 xylanase and a GH62 arabinofuranosidase, wherein the at least one polypeptide having cellobiohydrolase activity, endoglucanase activity, or beta-glucosidase activity comprises at least one, at least two, at least three, at least four, at least five polypeptides, or at least six polypeptides selected from the group consisting of: (i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1; (ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2; (iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3; (iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4; (v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and (vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

In an example embodiment, the fiber degrading enzyme blend comprises at least one polypeptide having cellobiohydrolase activity, endoglucanase activity, or beta-glucosidase activity, and a hemicellulolytic composition comprising a Talaromyces GH10 xylanase and a Talaromyces GH62 arabinofuranosidase, wherein the at least one polypeptide having cellobiohydrolase activity, endoglucanase activity, or beta-glucosidase activity comprises at least one, at least two, at least three, at least four, at least five polypeptides, or at least six polypeptides selected from the group consisting of: (i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1; (ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2; (iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3; (iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4; (v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and (vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

In an example embodiment, the fiber degrading enzyme blend comprises at least one polypeptide having cellobiohydrolase activity, endoglucanase activity, or beta-glucosidase activity, and a hemicellulolytic composition comprising a Talaromyces leycettanus GH10 xylanase and a Talaromyces pinophilus GH62 arabinofuranosidase, wherein the at least one polypeptide having cellobiohydrolase activity, endoglucanase activity, or beta-glucosidase activity comprises at least one, at least two, at least three, at least four, at least five polypeptides, or at least six polypeptides selected from the group consisting of: (i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1; (ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2; (iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3; (iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4; (v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and (vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

In an example embodiment, the fiber degrading enzyme blend comprises at least one polypeptide having cellobiohydrolase activity, endoglucanase activity, or beta-glucosidase activity, wherein the at least one polypeptide having cellobiohydrolase activity, endoglucanase activity, or beta-glucosidase activity comprises at least one, at least two, at least three, at least four, at least five polypeptides, or at least six polypeptides selected from the group consisting of: (i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1; (ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2; (iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3; (iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4; (v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and (vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6; and a hemicellulolytic composition comprising (i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and (ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

In an example embodiment, the fiber degrading enzyme blend comprises a cellulolytic composition and a hemicellulolytic composition, wherein the cellulolytic composition comprises two or three polypeptides selected from the group consisting of beta-glucosidase, cellobiohydrolase, and endoglucanase; and wherein the hemicellulolytic composition comprises an arabinofuranosidase and a xylanase.

In an example embodiment, the fiber degrading enzyme blend comprises a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises an arabinofuranosidase and a xylanase.

In an example embodiment, the fiber degrading enzyme blend comprises a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises a GH62 arabinofuranosidase and a GH10 xylanase.

In an example embodiment, the fiber degrading enzyme blend comprises a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises a Talaromyces GH62 arabinofuranosidase and a Talaromyces GH10 xylanase.

In an example embodiment, the fiber degrading enzyme blend comprises a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises a Talaromyces pinophilus GH62 arabinofuranosidase and a Talaromyces leycettanus GH10 xylanase.

In an example embodiment, the fiber degrading enzyme blend comprises a cellulolytic composition and a hemicellulolytic composition, wherein the cellulolytic composition comprises two or three polypeptides selected from the group consisting of beta-glucosidase, cellobiohydrolase I, and endoglucanase I; and wherein the hemicellulolytic composition comprises: (i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and (ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

In an example embodiment, the fiber degrading enzyme blend comprises a cellulolytic composition and a hemicellulolytic composition, wherein the cellulolytic composition comprises two or three polypeptides selected from the group consisting of an Aspergillus beta-glucosidase or a variant thereof; an Aspergillus cellobiohydrolase I; and a Trichoderma endoglucanase I; and wherein the hemicellulolytic composition comprises an arabinofuranosidase and a xylanase.

In an example embodiment, the fiber degrading enzyme blend comprises a cellulolytic composition and a hemicellulolytic composition, wherein the cellulolytic composition comprises two or three polypeptides selected from the group consisting of an Aspergillus beta-glucosidase or a variant thereof; an Aspergillus cellobiohydrolase I; and a Trichoderma endoglucanase I; and wherein the hemicellulolytic composition comprises an arabinofuranosidase and a xylanase, wherein the cellulolytic composition is derived from a strain of Trichoderma, such as Trichoderma reesei, or a strain of Humicola, such as Humicola insolens, or a strain of Chrysosporium, such as Chrysosporium lucknowense.

In an example embodiment, the fiber degrading enzyme blend comprises a cellulolytic composition and a hemicellulolytic composition, wherein the cellulolytic composition comprises two or three polypeptides selected from the group consisting of an Aspergillus fumigatus beta-glucosidase or a variant thereof; an Aspergillus fumigatus cellobiohydrolase I; and a Trichoderma reesei endoglucanase I; and wherein the hemicellulolytic composition comprises an arabinofuranosidase and a xylanase.

In an example embodiment, the fiber degrading enzyme blend comprises a cellulolytic composition and a hemicellulolytic composition, wherein the cellulolytic composition comprises two or three polypeptides selected from the group consisting of an Aspergillus fumigatus beta-glucosidase or a variant thereof; an Aspergillus fumigatus cellobiohydrolase I; and a Trichoderma reesei endoglucanase I; and wherein the hemicellulolytic composition comprises an arabinofuranosidase and a xylanase, wherein the cellulolytic composition is derived from a strain of Trichoderma, such as Trichoderma reesei, or a strain of Humicola, such as Humicola insolens, or a strain of Chrysosporium, such as Chrysosporium lucknowense.

In an example embodiment, the fiber degrading enzyme blend comprises a cellulolytic composition and a hemicellulolytic composition, wherein the cellulolytic composition comprises:
(i) an Aspergillus fumigatus beta-glucosidase disclosed in SEQ ID NO: 8 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 8;
(ii) a cellobiohydrolase I (CBH I), such as one derived from a strain of the genus Aspergillus, such as a strain of Aspergillus fumigatus, such as the CBHI disclosed as SEQ ID NO: 9, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 9; and
(iii) an endoglucanase I (EGI), such as one derived from a strain of the genus Trichoderma, such as a strain of Trichoderma reesei, such as the EGI disclosed as SEQ ID NO: 29, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 29; and
wherein the hemicellulolytic composition comprises an arabinofuranosidase and a xylanase.

In an example embodiment, the fiber degrading enzyme blend comprises a cellulolytic composition and a hemicellulolytic composition, wherein the cellulolytic composition comprises an Aspergillus beta-glucosidase or a variant thereof; an Aspergillus cellobiohydrolase I; and a Trichoderma endoglucanase I; and wherein the hemicellulolytic composition comprises an GH10 arabinofuranosidase and a GH10 xylanase.

In an example embodiment, the fiber degrading enzyme blend comprises a cellulolytic composition and a hemicellulolytic composition, wherein the cellulolytic composition comprises an Aspergillus beta-glucosidase or a variant thereof; an Aspergillus cellobiohydrolase I; and a Trichoderma endoglucanase I; and wherein the hemicellulolytic composition comprises a Talaromyces GH10 arabinofuranosidase and a Talaromyces GH10 xylanase.

In an example embodiment, the fiber degrading enzyme blend comprises a cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises an GH10 arabinofuranosidase and a GH10 xylanase.

In an example embodiment, the fiber degrading enzyme blend comprises a cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises a Talaromyces pinophilus GH10 arabinofuranosidase and a Talaromyces leycettanus GH10 xylanase.

In an example embodiment, the fiber degrading enzyme blend comprises a Trichoderma cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises a Talaromyces pinophilus GH10 arabinofuranosidase and a Talaromyces leycettanus GH10 xylanase.

In an example embodiment, the fiber degrading enzyme blend comprises a Trichoderma reesei cellulolytic composition and a hemicellulolytic composition, wherein the cellulolytic composition comprises an Aspergillus fumigatus beta-glucosidase or a variant thereof; an Aspergillus fumigatus cellobiohydrolase I; and a Trichoderma reesei endoglucanase I; and wherein the hemicellulolytic composition comprises a Talaromyces pinophilus GH10 arabinofuranosidase and a Talaromyces leycettanus GH10 xylanase.

In an example embodiment, the fiber degrading enzyme blend comprises a Trichoderma reesei cellulolytic composition and a hemicellulolytic composition, wherein the cellulolytic composition comprises:
(i) an Aspergillus fumigatus beta-glucosidase disclosed in SEQ ID NO: 8 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 8;
(ii) a cellobiohydrolase I (CBH I), such as one derived from a strain of the genus Aspergillus, such as a strain of Aspergillus fumigatus, such as the CBHI disclosed as SEQ ID NO: 9, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 9; and
(iii) an endoglucanase I (EGI), such as one derived from a strain of the genus Trichoderma, such as a strain of Trichoderma reesei, such as the EGI disclosed as SEQ ID NO: 29, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 29; and
wherein the hemicellulolytic composition comprises:
(i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and (ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

The fiber degrading enzyme blend can be used in saccharification, fermentation, or simultaneous saccharification and fermentation together with one or more additional enzymes. Any enzyme described herein for use in saccharification, fermentation, of SSF can be used in combination with the fiber degrading enzyme blend.

In an embodiment, any of the fiber degrading enzyme blends above further comprises a glucoamylase. Any glucoamylase described herein is contemplated for inclusion in the fiber degrading enzyme blends described herein.

In an embodiment, any of the fiber degrading enzyme blends above further comprises a trehalase. Any trehalase described herein is contemplated for inclusion in the fiber degrading enzyme blends described herein.

In an embodiment, any of the fiber degrading enzyme blends above further comprises an alpha-amylase. Any alpha-amylase described herein is contemplated for inclusion in the fiber degrading enzyme blends described herein.

In an embodiment, the fiber degrading enzyme blend further comprises a glucoamylase or glucoamylase blend. In an embodiment, the fiber degrading enzyme blend comprises the glucoamylase of SEQ ID NO: 13, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 13.

In an embodiment, the fiber degrading enzyme blend comprises a glucoamylase blend, the glucoamylase blend comprising the glucoamylase of SEQ ID NO: 13, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 13, and a trehalase wherein the trehalase is the trehalase of SEQ ID NO: 12, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 12, or the trehalase is SEQ ID NO: 28 or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 28.

In an embodiment, the fiber degrading enzyme blend comprises a glucoamylase blend, the glucoamylase blend comprising a glucoamylase, a trehalase and an alpha-amylase, wherein the glucoamylase is the glucoamylase of SEQ ID NO: 13, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 13, wherein the trehalase is the trehalase of SEQ ID NO: 12, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 12, or the trehalase is SEQ ID NO: 28 or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 28, and wherein the alpha-amylase is the Rhizomucor pusillus alpha-amylase with Aspergillus niger glucoamylase linker and SBD disclosed as SEQ ID NO: 11 with the following substitutions: G128D+D143N (activity ratio AGU:AGU:FAU(F): approx. 30:7:1) or one having at least 60%, at least 65%, at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 11.

### Polypeptides Having Cellulase Activity

Aspects described herein relate to polypeptide having cellobiohydrolase activity, endoglucanase activity, or beta-glucosidase activity. The present disclosure contemplates processes and enzyme blends or compositions comprising any polypeptides polypeptide having cellobiohydrolase activity, endoglucanase activity, or beta-glucosidase activity that when used alone, or in combination with each other or other enzymes or compositions described herein (e.g., cellulases/cellulolytic composition) result in an improvement in fermentation product yield (e.g., ethanol yield) compared to similar processes and/or enzyme blends or compositions lacking the polypeptides having cellobiohydrolase activity, endoglucanase activity, or beta-glucosidase activity.

Any polypeptide having polypeptide having cellobiohydrolase activity, endoglucanase activity, or beta-glucosidase activity described herein is also contemplated for expression by a fermenting organism or host cell.

In some embodiments, the present invention relates to isolated or purified polypeptides having a sequence identity of at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% or 100% identity to the mature polypeptide of SEQ ID NO: 1, which have cellobiohydrolase activity. In an aspect, the polypeptides differ by up to 10 amino acids, e.g., 1, 2, 3, 4, 5, 6, 7, 8, 9, or 10, from the mature polypeptide of SEQ ID NO: 1. The polypeptide preferably comprises, consists of, or consists essentially of the amino acid sequence of SEQ ID NO: 1 or the mature polypeptide thereof; or is a fragment thereof having cellobiohydrolase activity. In one aspect, the mature polypeptide is amino acids 19-520 of SEQ ID NO: 1.

In some embodiments, the present invention relates to isolated or purified polypeptides having a sequence identity of at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% or 100% identity to the mature polypeptide of SEQ ID NO: 2, which have cellobiohydrolase activity. In an aspect, the polypeptides differ by up to 10 amino acids, e.g., 1, 2, 3, 4, 5, 6, 7, 8, 9, or 10, from the mature polypeptide of SEQ ID NO: 2. The polypeptide preferably comprises, consists of, or consists essentially of the amino acid sequence of SEQ ID NO: 2 or the mature polypeptide thereof; or is a fragment thereof having cellobiohydrolase activity. In one aspect, the mature polypeptide is amino acids 1-503 of SEQ ID NO: 2.

In some embodiments, the present invention relates to isolated or purified polypeptides having a sequence identity of at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% or 100% identity to the mature polypeptide of SEQ ID NO: 3, which have cellobiohydrolase activity. In an aspect, the polypeptides differ by up to 10 amino acids, e.g., 1, 2, 3, 4, 5, 6, 7, 8, 9, or 10, from the mature polypeptide of SEQ ID NO: 3. The polypeptide preferably comprises, consists of, or consists essentially of the amino acid sequence of SEQ ID NO: 3 or the mature polypeptide thereof; or is a fragment thereof having cellobiohydrolase activity. In one aspect, the mature polypeptide is amino acids 26-533 of SEQ ID NO: 3.

In some embodiments, the present invention relates to isolated or purified polypeptides having a sequence identity of at least 70% identity, at least 71% identity, at least 72% identity, at identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% or 100% identity to the mature polypeptide of SEQ ID NO: 4, which have cellobiohydrolase activity. In an aspect, the polypeptides differ by up to 10 amino acids, e.g., 1, 2, 3, 4, 5, 6, 7, 8, 9, or 10, from the mature polypeptide of SEQ ID NO: 4. The polypeptide preferably comprises, consists of, or consists essentially of the amino acid sequence of SEQ ID NO: 4 or the mature polypeptide thereof; or is a fragment thereof having cellobiohydrolase activity. In one aspect, the mature polypeptide is amino acids 20-456 of SEQ ID NO: 4.

In some embodiments, the present invention relates to isolated or purified polypeptides having a sequence identity of at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% or 100% identity to the mature polypeptide of SEQ ID NO: 5, which have endoglucanase activity. In an aspect, the polypeptides differ by up to 10 amino acids, e.g., 1, 2, 3, 4, 5, 6, 7, 8, 9, or 10, from the mature polypeptide of SEQ ID NO: 5. The polypeptide preferably comprises, consists of, or consists essentially of the amino acid sequence of SEQ ID NO: 5 or the mature polypeptide thereof; or is a fragment thereof having endoglucanase activity. In one aspect, the mature polypeptide is amino acids 20-410 of SEQ ID NO: 5.

In some embodiments, the present invention relates to isolated or purified polypeptides having a sequence identity of at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% or 100% identity to the mature polypeptide of SEQ ID NO: 1, which have beta-glucosidase activity. In an aspect, the polypeptides differ by up to 10 amino acids, e.g., 1, 2, 3, 4, 5, 6, 7, 8, 9, or 10, from the mature polypeptide of SEQ ID NO: 6. The polypeptide preferably comprises, consists of, or consists essentially of the amino acid sequence of SEQ ID NO: 6 or the mature polypeptide thereof; or is a fragment thereof having beta-glucosidase activity. In one aspect, the mature polypeptide is amino acids 19-520 of SEQ ID NO: 6.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Trichoderma, Lecanicillium, Simplicillium, Aspergillus, Cornyascus, Acrophialophora, Rhinocladiella, Nemania, Talaromyces, Collariella, Rigidoporous, and/or Loramyces cell.

In another aspect, the cell is a Trichoderma harzianum, Trichoderma atroviride, Trichoderma reesei, Lecanicillium primulinum, Simplicillium lameillicola, Aspergillus nidulans, Aspergillus wentii, Cornyascus sepedonium, Acrophialophora fusispora, Rhinocladiella sp., Nemania serpens, Talaromyces leycettanus, Collariella virescens, Rigidoporous sp. 74222, and/or Loramyces macrosporus cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be polypeptide by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

The invention may further be described in the following numbered paragraphs:

Paragraph [1]. A process for producing a fermentation product, such as ethanol, from starch-containing material, the process comprising the steps of:
i) saccharifying the starch-containing material at a temperature below the initial gelatination temperature; and
ii) fermenting the saccharified starch-containing material using a fermenting organism;
wherein at least one polypeptide having cellobiohydrolase activity, endoglucanase activity, or beta-glucosidase activity is present or added during fermentation.

Paragraph [2]. A process for producing a fermentation product, such as ethanol, from starch-containing material, the process comprising the steps of:
i) liquefying the starch-containing material at a temperature above the initial gelatinization temperature using an alpha-amylase;
ii) saccharifying the liquefied starch-containing;
iii) fermenting saccharified starch-containing material using a fermenting organism;
wherein at least one polypeptide having cellobiohydrolase activity, endoglucanase activity, or beta-glucosidase activity is present or added during fermentation.

Paragraph [3]. The process of any of paragraphs [1] or [2], wherein at least one polypeptide having cellobiohydrolase activity is present or added during fermentation or simultaneous saccharification and fermentation.

Paragraph [4]. The process of any of paragraphs [1] or [2], wherein at least one polypeptide having endoglucanase activity is present or added during fermentation or simultaneous saccharification and fermentation.

Paragraph [5]. The process of any of paragraphs [1] or [2], wherein at least one polypeptide having beta-glucosidase activity is present or added during fermentation or simultaneous saccharification and fermentation.

Paragraph [6]. The process of any of paragraphs [1] to [5], wherein the at least one polypeptide having cellobiohydrolase activity, endoglucanase activity, or beta-glucosidase activity comprises at least one, at least two, at least three, at least four, at least five polypeptides, or at least six polypeptides selected from the group consisting of:
(i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1;
(ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2;
(iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3;
(iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4;
(v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and
(vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

Paragraph [7]. The process of any of paragraphs [1] to [6], wherein the at least one polypeptide having cellobiohydrolase activity, endoglucanase activity, or beta-glucosidase activity comprises or consists of SEQ ID NO: 1, SEQ ID NO: 2, SEQ ID NO: 3, SEQ ID NO: 4, SEQ ID NO: 5, SEQ ID NO: 6, or the mature polypeptide of SEQ ID NO: 1, SEQ ID NO: 2, SEQ ID NO: 3, SEQ ID NO: 4, SEQ ID NO: 5, SEQ ID NO: 6.

Paragraph [8]. The process of any one of paragraphs [1] to [7], wherein the mature polypeptide is amino acids 19 to 520 of SEQ ID NO: 1, amino acids 1 to 503 of SEQ ID NO: 2, amino acids 26 to 533 of SEQ ID NO: 3, amino acids 20 to 456 of SEQ ID NO: 4, amino acids 20 to 410 of SEQ ID NO: 5 and amino acids 20 to 855 of SEQ ID NO: 6.

Paragraph [9]. The process of any of paragraphs [1] to [8], wherein the at least one polypeptide having cellobiohydrolase activity, endoglucanase activity, or beta-glucosidase activity is a variant of the mature polypeptide of SEQ ID NO: 1, SEQ ID NO: 2, SEQ ID NO: 3, SEQ ID NO: 4, SEQ ID NO: 5, SEQ ID NO: 6 comprising a substitution, deletion, and/or insertion at one or more (e.g., several) positions.

Paragraph [10]. The process of any of paragraphs [1] to [8], wherein the at least one polypeptide having cellobiohydrolase activity, endoglucanase activity, or beta-glucosidase activity is a fragment of SEQ ID NO: 1, SEQ ID NO: 2, SEQ ID NO: 3, SEQ ID NO: 4, SEQ ID NO: 5, SEQ ID NO: 6, wherein the fragment has cellobiohydrolase activity, endoglucanase activity, or beta-glucosidase activity.

Paragraph [11]. The process of any of paragraphs [1] to [10], wherein the at least one polypeptide having cellobiohydrolase activity, endoglucanase activity, or beta-glucosidase activity are dosed in the range 0.1-1000 micro gram EP/g DS; 0.5-500 micro gram EP/g DS; 1-100 micro gram EP/g DS; such as 5-50 micro gram EP/g DS.

Paragraph [12]. The process of any of paragraphs [1] to [11], wherein saccharification and/or fermentation is performed in the presence of at least one additional cellulase in the form of a cellulolytic composition, for example, a cellulolytic composition that comprises the at least one polypeptide and the at least one additional cellulase.

Paragraph [13]. The process of paragraph [12], wherein the cellulolytic composition is derived from a strain of Trichoderma, such as Trichoderma reesei, or a strain of Humicola, such as Humicola insolens, or a strain of Chrysosporium, such as Chrysosporium lucknowense.

Paragraph [14]. The process of paragraph [12] or [13], wherein the cellulolytic composition comprises two or three polypeptides selected from the group consisting of:
beta-glucosidase;
cellobiohydrolase I; and
endoglucanase I.

Paragraph [15]. The process of any one of paragraphs [12] to [14], wherein the cellulolytic composition comprises two or three of the following components:
(i) an Aspergillus fumigatus beta-glucosidase or a variant thereof;
(ii) an Aspergillus fumigatus cellobiohydrolase I; and
(iii) a Trichoderma reesei endoglucanase I.

Paragraph [16]. The process of any one of paragraphs [12] to [15], wherein the cellulolytic composition is a Trichoderma reesei cellulolytic composition further comprising:
(i) an Aspergillus fumigatus beta-glucosidase disclosed in SEQ ID NO: 8 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 8;
(ii) a cellobiohydrolase I (CBH I), such as one derived from a strain of the genus Aspergillus, such as a strain of Aspergillus fumigatus, such as the CBHI disclosed as SEQ ID NO: 9, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 9; and
(iii) an endoglucanase I (EGI), such as one derived from a strain of the genus Trichoderma, such as a strain of Trichoderma reesei, such as the EGI disclosed as SEQ ID NO: 29, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 29.

Paragraph [17]. The process of any of paragraphs [12] to [16], wherein saccharification and/or fermentation is performed in the presence of a hemicellulolytic composition comprising a xylanase and an arabinofuranosidase.

Paragraph [18]. The process of any of paragraphs [12] to [17], wherein the hemicellulolytic composition comprises:
(i) a GH10 xylanase; and
(ii) a GH62 arabinofuranosidase.

Paragraph [19]. The process of any one of paragraphs [12] to [18], wherein the hemicellulolytic composition comprises:
(i) a GH10 xylanase from Talaromyces leycettanus; and
(ii) a GH62 arabinofuranosidase from Talaromyces pinophilus.

Paragraph [20]. The process of any one of paragraphs [12] to [19], wherein the hemicellulolytic composition comprises:
(i) a GH10 xylanase, such as one derived from a strain of the genus Talaromyces, such as a strain of Talaromyces leycettanus, such as the one disclosed as SEQ ID NO: 59, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 59; and
(ii) a GH62 arabinofuranosidase, such as one derived from a strain of the genus Talaromyces, such as a strain of Talaromyces pinophilus, such as the one disclosed as SEQ ID NO: 60, or one having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 60.

Paragraph [21]. The process of any one of paragraphs [12] to [20], wherein saccharification and/or fermentation is performed in the presence of the cellulolytic composition of any one of paragraphs to and the hemicellulolytic composition of any one of paragraphs [2] to [20].

Paragraph [22]. The process of any of paragraphs [2] to [21], wherein liquefaction is performed in the presence of a protease having a thermostability value of more than 20% determined as Relative Activity at 80° C./70° C.

Paragraph [23]. The process of any of paragraphs [2] to [22], wherein liquefaction is performed in the presence of a glucoamylase.

Paragraph [24]. The process of any of paragraphs [1] to [23], wherein the carbohydrate-source generating enzyme(s) is at least a glucoamylase and optionally in combination with a fungal acid alpha-amylase.

Paragraph [25]. The process of any of paragraphs [1] to [24], wherein the fermentation product is an alcohol, preferably ethanol, especially fuel ethanol, potable ethanol and/or industrial ethanol.

Paragraph [26]. The process of any of paragraphs [2] to [25], wherein the alpha-amylase is a bacterial or fungal alpha-amylase.

Paragraph [27]. The process of any of paragraphs [2] to [26], wherein the alpha-amylase is from the genus Bacillus, such as a strain of Bacillus stearothermophilus, in particular a variant of a Bacillus stearothermophilus alpha-amylase, such as the one shown in SEQ ID NO: 14, or alpha-amylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 30.

Paragraph [28]. The process of paragraph [27], wherein the Bacillus stearothermophilus alpha-amylase comprises a deletion of two amino acids in the region corresponding to positions 179-182 using SEQ ID NO: 14 for numbering.

Paragraph [29]. The process of paragraph [28], wherein the deletion is selected from the group consisting of 179*+180*, 179*+181*, 179*+182*, 180*+181*, 180*+182*, and 181*+182*, particularly I181*+G182*.

Paragraph [30]. The process according to any of paragraphs [2] to [29], wherein the alpha-amylase comprises a substitution N193F using SEQ ID NO: 14 for numbering.

Paragraph [31]. The process of any of paragraphs [2] to [29], wherein the Bacillus stearothermophilus alpha-amylase has a substitution in position S242, preferably S242Q substitution using SEQ ID NO: 14 for numbering.

Paragraph [32]. The process of any of paragraphs [27] to [31], wherein the Bacillus stearothermophilus alpha-amylase has a substitution in position E188, preferably E188P substitution using SEQ ID NO: 14 for numbering.

Paragraph [33]. The process of any of paragraphs [2] to [32], wherein the alpha-amylase has a T½ (min) at pH 4.5, 85° C., 0.12 mM CaCl₂)) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70.

Paragraph [34]. The process of any of paragraphs [2] to [33], wherein the alpha-amylase is selected from the following group of Bacillus stearothermophilus alpha-amylase variants (using SEQ ID NO: 14 for numbering):
I181*+G182*+N193F+E129V+K177L+R179E;
I181*+G182*+N193F+V59A+Q89R+E129V+K177L+R179E+H208Y+K220P+N224L+Q254S
I181*+G182*+N193F+V59A+Q89R+E129V+K177L+R179E+Q254S+M284V;
I181*+G182*+N193F+V59A+E129V+K177L+R179E+Q254S+M284V;
I181*+G182*+N193F+E129V+K177L+R179E+K220P+N224L+S242Q+Q254S;
I181*+G182*+V59A+E129V+K177L+R179E+Q254S+M284V+V212T+Y268G+N293Y+T297N;
I181*+G182*+V59A+E129V+K177L+R179E+Q254S+M284V+V212T+Y268G+N293Y+T297N+S173N+E188P+H208Y+S242Y+K2791;
   |181*+G182*+V59A+E129V+K177L+R179S+Q254S+M284V+V212T+Y268G+N293Y+T297N+A184Q+E188P+T191N
I181*+G182*+V59A+E129V+K177L+R179S+Q254S+M284V+V212T+Y268G+N293Y+T297N+A184Q+E188P+T191N+S242Y+K2791;
I181*+G182*+V59A+E129V+K177L+R179E+Q254S+M284V+V212T+Y268G+N293Y+T297N+E188P+K279W;
I181*+G182*+V59A+E129V+K177L+R179E+Q254S+M284V+V212T+Y268G+N293Y+T297N+W115D+D117Q+T133P; and
wherein the variant has at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 14.

Paragraph [35]. The process of any of paragraphs [2] to [34], wherein a protease with a thermostability value of more than 25% determined as Relative Activity at 80° C./70° C. is present in liquefaction step i).

Paragraph [36]. The process of paragraph [35], wherein the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

Paragraph [37]. The process of paragraph [35] or [36], wherein the protease has a thermostability of between 20% and 50%, such as between 20% and 40%, such as 20% and 30% determined as Relative Activity at 80° C./70° C.

Paragraph [38]. The process of any of paragraphs [35] to [37], wherein the protease has a thermostability between 50% and 115%, such as between 50% and 70%, such as between 50% and 60%, such as between 100% and 120%, such as between 105% and 115% determined as Relative Activity at 80° C./70° C.

Paragraph [39]. The process of any of paragraphs [35] to [38], wherein the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

Paragraph [40]. The process of any of paragraphs [35] to [39], wherein the protease has thermostability of between 10% and 50%, such as between 10% and 30%, such as between 10% and 25% determined as Relative Activity at 85° C./70° C.

Paragraph [41]. The process of any of paragraphs [35] to [40], wherein the protease has a thermostability above 60%, such as above 90%, such as above 100%, such as above 110% at 85° C. as determined using the Zein-BCA assay.

Paragraph [42]. The process of any of paragraphs [35] to [41], wherein the protease has a thermostability between 60-120, such as between 70-120%, such as between 80-120%, such as between 90-120%, such as between 100-120%, such as 110-120% at 85° C. as determined using the Zein-BCA assay.

Paragraph [43]. The process of any of paragraphs [35] to [42], wherein the protease is of fungal or bacterial origin.

Paragraph [44]. The process of any of paragraphs [35] to [43], wherein the protease is a metallo protease or a serine protease.

Paragraph [45]. The process of paragraph [44], wherein the protease is a variant of the metallo protease derived from a strain of the genus Thermoascus, preferably a strain of Thermoascus aurantiacus, especially Thermoascus aurantiacus CGMCC No. 0670.

Paragraph [46]. The process of paragraph [45], wherein the protease is a variant of the metallo protease disclosed as SEQ ID NO: 15 with the following mutations:
D79L+S87P+A112P+D142L;
D79L+S87P+D142L; or
A27K+D79L+Y82F+S87G+D104P+A112P+A126V+D142L; and
   wherein the protease has at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 15.

Paragraph [47]. The process of paragraph [45], wherein the protease is a serine protease, particularly an S8 serine protease derived from a strain of Pyrococcus, preferably a strain of Pyrococcus furiosus, or derived from a strain of Thermococcus, preferably Themococcus thioreducens or Thermococcus nautili, or derived from a strain of Palaeococcus, preferably Palaeococcus ferrophilus.

Paragraph [48]. The process of paragraph [47], wherein the protease is derived from a strain of Pyrococcus, preferably a strain of Pyrococcus furiosus.

Paragraph [49]. The process of paragraph [48], wherein the protease is the one shown in SEQ ID NO: 16, or a protease having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 16.

Paragraph [50]. The process of any of paragraphs [35] to [49], wherein the protease is derived from a strain of Thermobifida, preferably a strain of Thermobifida cellulosytica.

Paragraph [51]. The process of paragraph [50], wherein the protease is the one shown in SEQ ID NO: 18, or a protease having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 18.

Paragraph [52]. The process of any of paragraphs [1] to [51], wherein a glucoamylase is present and/or added during saccharification and/or fermentation.

Paragraph [53]. The process of paragraph [52], wherein the glucoamylase present and/or added during saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably Talaromyces emersonii, or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

Paragraph [54]. The process of paragraphs [1] or [53], wherein the glucoamylase present and/or added during saccharification and/or fermentation is a blend comprising Talaromyces emersonii glucoamylase of SEQ ID NO: 27, or glucoamylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 27, a Trametes cingulata glucoamylase of SEQ ID NO: 24, or glucoamylase having at least 70% identity, at least 71% identity, at least 72% identity, at identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 24, and a Rhizomucor pusillus alpha-amylase with an Aspergillus niger glucoamylase linker and starch-binding domain (SBD), of SEQ ID NO: 11, and comprising the following substitutions: G128D+D143N, and having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 40.

Paragraph [55]. The process of any of paragraphs [52] to [54], wherein the glucoamylase present and/or added during saccharification and/or fermentation is a blend comprising Gloeophyllum sepiarium glucoamylase shown as SEQ ID NO: 13, or a glucoamylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 13, and an alpha-amylase from Rhizomucor pusillus with an Aspergillus niger glucoamylase linker and starch-binding domain (SBD), disclosed SEQ ID NO: 11 with the following substitutions: G128D+D143N, and having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 40.

Paragraph [56]. The process of any one of paragraphs [1] to [55], wherein a trehalase is present and/or added during saccharification and/or fermentation.

Paragraph [57]. The process of paragraph [56], wherein the trehalase present and/or added during saccharification and/or fermentation is a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to the mature polypeptide of SEQ ID NO: 12 and having trehalase activity.

Paragraph [58]. The process of paragraph [56], wherein the trehalase present and/or added during saccharification and/or fermentation is a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to the mature polypeptide of SEQ ID NO: 28 and having trehalase activity.

Paragraph [59]. The process of any of paragraphs [1] to [58], wherein saccharification and fermentation are carried out sequentially or simultaneously.

Paragraph [60]. The process of any of paragraphs [1] to [59], wherein fermentation or simultaneous saccharification and fermentation (SSF) are carried out at a temperature from 25° C. to 40° C., such as from 28° C. to 35° C., such as from 30° C. to 34° C., preferably around about 32° C.

Paragraph [61]. The process of any of paragraphs [1] to [60], wherein the fermentation product is recovered after fermentation, such as by distillation.

Paragraph [62]. The process of any of paragraphs [1] to [61], wherein the starch-containing starting material is whole grains.

Paragraph [63]. The process of any of paragraphs [1] to [62], wherein the starch-containing material is derived from corn, wheat, barley, rye, milo, sago, cassava, manioc, tapioca, sorghum, rice or potatoes.

Paragraph [64]. The process of any of paragraphs [1] to [63], wherein the organism applied in fermentation is a yeast, particularly a Saccharomyces spp., more particular Saccharomyces cerevisiae.

Paragraph [65]. An isolated polypeptide having cellobiohydrolase activity, endoglucanase activity, or beta-glucosidase activity selected from the group consisting of:
(i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1;
(ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2;
(iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3;
(iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4;
(v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and
(vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

Paragraph [66]. The polypeptide of paragraph [65], wherein the amino acid sequence comprises or consists of SEQ ID NO: 1, SEQ ID NO: 2, SEQ ID NO: 3, SEQ ID NO: 4, SEQ ID NO: 5, SEQ ID NO: 6, or the mature polypeptide of SEQ ID NO: 1, SEQ ID NO: 2, SEQ ID NO: 3, SEQ ID NO: 4, SEQ ID NO: 5, SEQ ID NO: 6.

Paragraph [67]. The polypeptide of paragraph [65] or [66], wherein the mature polypeptide is amino acids 19 to 520 of SEQ ID NO: 1, amino acids 1 to 503 of SEQ ID NO: 2, amino acids 26 to 533 of SEQ ID NO: 3, amino acids 20 to 456 of SEQ ID NO: 4, amino acids 20 to 410 of SEQ ID NO: 5 and amino acids 20 to 855 of SEQ ID NO: 6.

Paragraph [68]. The polypeptide of paragraph [65], which is a variant of the mature polypeptide of SEQ ID NO: 1, SEQ ID NO: 2, SEQ ID NO: 3, SEQ ID NO: 4, SEQ ID NO: 5, SEQ ID NO: 6 comprising a substitution, deletion, and/or insertion at one or more (e.g., several) positions.

Paragraph [69]. The polypeptide of paragraph [65], which is a fragment of SEQ ID NO: 1, SEQ ID NO: 2, SEQ ID NO: 3, SEQ ID NO: 4, SEQ ID NO: 5, SEQ ID NO: 6, wherein the fragment has cellobiohydrolase activity, endoglucanase activity, or beta-glucosidase activity.

Paragraph [70]. An enzyme blend or enzyme composition comprising at least one polypeptide having cellobiohydrolase activity, endoglucanase activity, or beta-glucosidase activity of any one of paragraphs [65] to [70].

Paragraph [71]. The blend or composition of paragraph [70], further comprising a carbohydrate-source generating enzyme, particularly a glucoamylase.

Paragraph [72]. The blend or composition of paragraph [70] or [71], further comprising a cellulase/cellulolytic composition comprising a beta-glucosidase, a cellobiohydrolase and an endoglucanase.

Paragraph [73]. The blend or composition of paragraph [72], wherein the cellulases/cellulolytic composition comprises one or more polypeptides selected from the group consisting of:
GH61 polypeptide having cellulolytic enhancing activity,
beta-glucosidase;
Cellobiohydrolase I;
Cellobiohydrolase II;
or a mixture of two, three, or four thereof.

Paragraph [74]. The blend or composition of paragraph [72] or [73], wherein the cellulases/cellulolytic composition comprises one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

Paragraph [75]. The blend or composition of any one of paragraphs [70] to [74], further comprising a trehalase.

Paragraph [76]. An isolated polynucleotide encoding the polypeptide of any of paragraphs [65] to [69].

Paragraph [77]. A nucleic acid construct or expression vector comprising the polynucleotide of paragraph [76] operably linked to one or more control sequences that direct the production of the polypeptide in an expression host.

Paragraph [78]. A recombinant host cell comprising the polynucleotide of paragraph operably linked to one or more control sequences that direct the production of the polypeptide.

Paragraph [79]. A method of producing a polypeptide having cellobiohydrolase activity, endoglucanase activity, or beta-glucosidase activity, comprising:
(a) cultivating the host cell of paragraph under conditions conducive for production of the polypeptide; and
(b) recovering the polypeptide.

Paragraph [80]. A transgenic plant, plant part or plant cell transformed with a polynucleotide encoding the polypeptide of any of paragraphs [65] to [69].

Paragraph [81]. A method of producing a polypeptide having cellobiohydrolase activity, endoglucanase activity, or beta-glucosidase activity, comprising:
(a) cultivating the transgenic plant or plant cell of paragraph under conditions conducive for production of the polypeptide; and
(b) recovering the polypeptide.

Paragraph [82]. A method of producing a mutant of a parent cell, comprising inactivating a polynucleotide encoding the polypeptide of any of paragraphs [65] to [69], which results in the mutant producing less of the polypeptide than the parent cell.

Paragraph [83]. A mutant cell produced by the method of paragraph [82].

Paragraph [84]. A method of producing a protein, comprising:
(a) cultivating the mutant cell of paragraph under conditions conducive for production of the protein; and
(b) recovering the protein.

Paragraph [85]. A recombinant host cell comprising at least one heterologous polynucleotide encoding a polypeptide having cellobiohydrolase activity, endoglucanase activity, or beta-glucosidase activity selected from the group consisting of:
(i) the mature polypeptide of SEQ ID NO: 1, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, or at least 99% identity to SEQ ID NO: 1;
(ii) the mature polypeptide of SEQ ID NO: 2, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 2;
(iii) the mature polypeptide of SEQ ID NO: 3, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 3;
(iv) the mature polypeptide of SEQ ID NO: 4, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity or at least 99% identity to SEQ ID NO: 4;
(v) the mature polypeptide of SEQ ID NO: 5, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 5; and
(vi) the mature polypeptide of SEQ ID NO: 6, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 6.

Paragraph [86]. The recombinant host cell of paragraph [85], wherein the at least one heterologous polynucleotide encoding a polypeptide having cellobiohydrolase activity, endoglucanase activity, or beta-glucosidase activity comprises or consists of SEQ ID NO: 1, SEQ ID NO: 2, SEQ ID NO: 3, SEQ ID NO: 4, SEQ ID NO: 5, SEQ ID NO: 6, or the mature polypeptide of SEQ ID NO: 1, SEQ ID NO: 2, SEQ ID NO: 3, SEQ ID NO: 4, SEQ ID NO: 5, SEQ ID NO: 6.

Paragraph [87]. The recombinant host cell of paragraphs [85] or [86], wherein the mature polypeptide is amino acids 19 to 520 of SEQ ID NO: 1, amino acids 1 to 503 of SEQ ID NO: 2, amino acids 26 to 533 of SEQ ID NO: 3, amino acids 20 to 456 of SEQ ID NO: 4, amino acids 20 to 410 of SEQ ID NO: 5 and amino acids 20 to 855 of SEQ ID NO: 6.

Paragraph [88]. The recombinant host cell of paragraph [85], wherein the at least one heterologous polynucleotide encoding a polypeptide having cellobiohydrolase activity, endoglucanase activity, or beta-glucosidase activity is a variant of the mature polypeptide of SEQ ID NO: 1, SEQ ID NO: 2, SEQ ID NO: 3, SEQ ID NO: 4, SEQ ID NO: 5, SEQ ID NO: 6 comprising a substitution, deletion, and/or insertion at one or more (e.g., several) positions.

Paragraph [89]. The recombinant host cell of paragraph [85], wherein the at least one heterologous polynucleotide encoding a polypeptide having cellobiohydrolase activity, endoglucanase activity, or beta-glucosidase activity is a fragment of SEQ ID NO: 1, SEQ ID NO: 2, SEQ ID NO: 3, SEQ ID NO: 4, SEQ ID NO: 5, SEQ ID NO: 6, wherein the fragment has cellobiohydrolase activity, endoglucanase activity, or beta-glucosidase activity.

Paragraph [90]. The recombinant host cell of any of paragraphs [85] to [89], wherein the cell further comprises a heterologous polynucleotide encoding a glucoamylase.

Paragraph [91]. The recombinant host cell of paragraph [90], wherein the heterologous polynucleotide encoding the glucoamylase is operably linked to a promoter that is foreign to the polynucleotide.

Paragraph [92]. The recombinant host cell of any of paragraphs [85] to [91], wherein the cell further comprises a heterologous polynucleotide encoding an alpha-amylase.

Paragraph [93]. The recombinant host cell of paragraph [92], wherein the heterologous polynucleotide encoding the alpha-amylase is operably linked to a promoter that is foreign to the polynucleotide.

Paragraph [94]. The recombinant host cell of any of paragraphs [85] to [93], wherein the cell further comprises a heterologous polynucleotide encoding a protease.

Paragraph [95]. The recombinant host cell of paragraph [94], wherein the heterologous polynucleotide encoding the protease is operably linked to a promoter that is foreign to the polynucleotide.

Paragraph [96]. The recombinant host cell of any of paragraphs [85] to [95], wherein the cell further comprises a disruption to an endogenous gene encoding a glycerol 3-phosphate dehydrogenase (GPD).

Paragraph [97]. The recombinant host cell of any of paragraphs [85] to [96], wherein the cell further comprises a disruption to an endogenous gene encoding a glycerol 3-phosphatase (GPP).

Paragraph [98]. The recombinant host cell of any of paragraphs [85] to [97], wherein the cell is a yeast cell.

Paragraph [99]. The recombinant host cell of any of paragraphs [85] to [98], wherein the cell is a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell.

Paragraph [100]. The recombinant host cell of any of paragraphs [85] to [99], wherein the cell is a Saccharomyces cerevisiae cell.

Paragraph [101]. A composition comprising the recombinant host cell of any of paragraphs [85] to [100] and one or more naturally occurring and/or non-naturally occurring components, such as components are selected from the group consisting of: surfactants, emulsifiers, gums, swelling agents, and antioxidants.

Paragraph [102]. Use of a recombinant host cell of any of paragraphs [85] to [101] in the production of ethanol.

The invention described and claimed herein is not to be limited in scope by the specific aspects or embodiments herein disclosed, since these aspects/embodiments are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. All references are specifically incorporated by reference for that which is described.

The following examples are offered to illustrate certain aspects/embodiments of the present invention, but not in any way intended to limit the scope of the invention as claimed.

### EXAMPLES

### Enzymes Used in the Examples:

Alpha-Amylase 369 (AA369): Bacillus stearothermophilus alpha-amylase of SEQ ID NO: 14 with the mutations: I181*+G182*+N193F+V59A+Q89R+E129V+K177L+R179E+Q254S+M284V truncated to 491 amino acids.

Protease PfuS: Protease derived from Pyrococcus furiosus shown in SEQ ID NO: 16 herein

Alpha-amylase blend X: Blend of Amylase AA369 and Protease PfuS.

Trehalase Ms: Myceliophthora sepedonium trehalase disclosed herein as SEQ ID NO:28.

Trehalase Tf: Talaromyces funiculosus trehalase disclosed herein as SEQ ID NO: 12.

Glucoamylase Blend A: Blend comprising Gloeophyllum sepiarium glucoamylase disclosed in SEQ ID NO: 13 and Trehalase Ms.

Glucoamylase Blend B: Blend comprising Gloeophyllum sepiarium glucoamylase disclosed in SEQ ID NO: 13, Trehalase Tf and Rhizomucor pusillus alpha-amylase with Aspergillus niger glucoamylase linker and SBD disclosed as SEQ ID NO: 11 with the following substitutions: G128D+D143N (activity ratio AGU:AGU:FAU(F): approx. 30:7:1).

Cellulolytic Composition: Cellulolytic composition derived from Trichoderma reesei comprising Aspergillus fumigatus beta-glucosidase (SEQ ID NO: 2 in WO 2005/047499 SEQ ID NO: 8 herein) variant F100D, S283G, N456E, F512Y) disclosed in WO 2012/044915; Aspergillus fumigatus Cel7A CBH1 disclosed as SEQ ID NO: 6 in WO2011/057140 (SEQ ID NO: 9 herein) and Trichoderma reesei endoglucanase 1 (Tr EG1) disclosed as SEQ ID NO: 29 herein.

GH10 xylanase: Talaromyces leycettanus xylanase disclosed herein as SEQ ID NO: 59.

GH62 arabinofuranosidase: Talaromyces pinophilus arabinofuranosidase disclosed herein as SEQ ID NO: 60.

Hemicellulolytic Composition: composition comprising GH10 xylanase and GH62 arabinofuranosidase.

### Media and Solutions:

The following media or solutions were autoclaved at 121° C. for 20 minutes unless otherwise specified. Chemicals used as buffers and substrates were commercial products of at least reagent grade.

PDA plate was composed of 39 g of potato dextrose agar in a final volume of 1 L with deionized water.

YG plate was composed of 5 g of yeast extract, 10 g of glucose and 20 g of agar, in a final volume 1 L with deionized water.

YPG medium was composed of 0.4% yeast extract, 0.1% KH₂PO₄, 0.05% MgSO₄·7H₂O and 1.5% glucose in deionized water.

LB medium was composed of 10 g of Bacto-tryptone, 5 g of yeast extract and 5 g of sodium chloride, in a final volume of 1 L with deionized water.

LBA medium (LB+Ampicillin) was prepared by adding 100 mg/ml Ampicillin to LB medium at 1:1000 at room temperature.

LBA plate was prepared by adding 15 g of agar to LB medium in a final volume of 1 L prior autoclaving, and adding 100 mg/ml Ampicillin at 1:1000 when cooling to 60° C.

Minimal medium plate was composed of 342 g of sucrose, 20 g of agar powder and 20 ml of COVE salt solution, in a final volume of 1 L with deionized water. After autoclaving, the medium was then supplemented with 10 ml of 1M Acetamide (filter sterilized) when cooling to 60° C.

COVE salt solution was composed of 26 g of MgSO₄·7H₂O, 26 g of KCL, 76 g of KH₂PO₄ and 50 ml of COVE trace metal solution, in a final volume of 1 L with deionized water.

COVE trace metal solution was composed of 0.04 g of Na₂B₄O₇·10H₂O, 0.4 g of CuSO₄·5H₂O, 0.8 g of FeSO₄·7H₂O, 0.8 g of MnSO₄·H₂O, 0.8 g of Na₂MoO₄·2H₂O and 8 g of ZnSO₄·7H₂O, in a final volume of 1 L with deionized water.

TOP agar medium was composed of 6 g SeaKem GTG agarose, 20 ml of COVE salt solution and 342 g sucrose, in a final volume of 1 L with deionized water. After autoclaving, the medium was then supplemented with 10 ml of 1M Acetamide when cooling to 60° C.

COVE medium reisolation plate was composed of 30 g of sucrose, 20 ml of COVE salt solution and 20 g of agar, in a final volume of 1 L with deionized water. After autoclaving, the medium was then supplemented with 100 μl of Triton X-100 and 10 ml of 1M Acetamide when cooling to 60° C.

COVE medium slant was composed of 30 g of sucrose, 20 ml of COVE salt solution and 20 g of agar, in a final volume up to 1 L with deionized water. After autoclaving, the medium was supplemented with 10 ml of 1M Acetamide when cooling to 60° C.

60% PEG4000 (W/V) was prepared by dissolving 60 g of PEG4000 in 40 ml of deionized water and then adding CaCl₂ and Tris/HCl pH7.5 to a final concentration of 10 mM each. The final volume was adjusted to 1 L with deionized water.

DAP4C medium was composed of 0.5 g yeast extract, 10 g maltose, 20 g glucose, 11 g MgSO₄·7H₂O, 1 g KH₂PO₄, 2.2 g Citric acid·H₂O, 5.2 g K₃PO₄H₂O and 0.5 ml of AMG Trace element solution, in a final volume of up to 1 L with deionized water, dissolving by stirring. Then 400 ml was aliquoted to a shake flask of 2 L. One tablet of 0.5 g Calcium carbonate was added to each flask. After autoclaving, 3.3 ml of 20% lactic acid (autoclaved) and 9.3 ml of 50% (NH₄)₂HPO₄ (filter sterilized) were added to each flask.

AMG Trace element solution was composed of 6.8 g ZnCl₂, 2.5 g CuSO₄·5H₂O, 0.24 g NiCl₂·5H₂O, 13.9 g FeSO₄·7H₂O, 13.6 g MnSO₄·5H₂O and 3 g Citric acid·H₂O, in a final volume of 1 L with deionized water.

YPM medium was composed of 10 g of yeast extract, 20 g of peptone and 20 g of maltose, in a final volume 1 L with deionized water.

### Strains:

The fungal strain NN053878 was obtained from the Institute of Microbiology, Chinese Academy of Sciences, and identified as Penicillium swiecickii, based on both morphological characteristics and ITS rDNA sequence.

The fungal strain NN046799 was isolated from a soil sample collected in China, by dilution plate method with YG medium, pH7, 37° C. The strain was then purified by transferring a single conidium onto a PDA agar plate, and identified as Talaromyces verruculosus, based on both morphological characteristics and ITS rDNA sequence.

The fungal strain NN046877 was isolated from a soil sample collected in China, by dilution plate method with YG medium, pH7, 37° C. The strain was then purified by transferring a single conidium onto a PDA agar plate, and identified as Talaromyces pinophilus, based on both morphological characteristics and ITS rDNA sequence.

The fungal strain NN058608 was purchased from Centraalbureau voor Schimmelcultures (CBS, Utrecht, Netherlands) with access number of CBS690.92. The strain NN058608 was identified as Cladosporium antarcticum, based on both morphological characteristics and ITS rDNA sequence.

Escherichia coli Top10 strain was purchased from TIANGEN (TIANGEN Biotech Co. Ltd., Beijing, China) and used to propagate the expression vectors.

Aspergillus oryzae strain MT3568 was described in WO2014026630A1.

Aspergillus oryzae strain DAu785 was described in WO2018113745.

Aspergillus oryzae strain HowB101 was described in WO9535385.

Yeast strain MEJI797 is MBG5012 of WO2019/161227 further expressing a Pycnopous sanguineus glucoamylase (SEQ ID NO: 4 of WO2011/066576) and a hybrid Rhizomucor pusillus alpha amylase expression cassette (as described in WO2013/006756).

### Example 1: Cloning and Identification of Penicillium swiecickii CBHI

### Genomic DNA Extraction

The Penicillium swiecickii strain NN053878 was inoculated onto a PDA plate and incubated for 7 days at 20° C. in darkness. Several mycelia-PDA plugs were inoculated into 500 ml shake flasks containing 100 ml of YPG medium. The flasks were incubated for 4 days at 20° C. with shaking at 160 rpm.

The mycelia were collected by filtration through MIRACLOTH® and frozen under liquid nitrogen. Frozen mycelia were ground, by a mortar and a pestle, to a fine powder, and genomic DNA was isolated using DNeasy® Plant Maxi Kit (24) (QIAGEN GmbH, Hilden, Germany) following the manufacturer's instruction.

### Genome Sequencing, Assembly and Annotation

The extracted genomic DNA sample of NN053878 was delivered to Fasteris (Switzerland) for genome sequencing using an ILLUMINA® HiSeq 2000 System (Illumina, Inc., San Diego, CA, USA). The raw reads were assembled at Novozymes Denmark using program Idba (Peng, Yu et al., 2010, Research in Computational Molecular Biology, 6044:426-440. Springer Berlin Heidelberg). The assembled sequences were analyzed using standard bioinformatics methods for gene identification and function prediction. GeneMark-ES fungal version (Ter-Hovhannisyan V et al., 2008, Genome Research 18(12): 1979-1990) was used for gene prediction. Blastall version 2.2.25 (Altschul et al., 1990, Journal of Molecular Biology, 215(3): 403-410) and HMMER version 2.1.1 (National Center for Biotechnology Information (NCBI), Bethesda, MD, USA) were used to predict function based on structural homology. The Penicillium swiecickii GH7 cellobiohydrolase I (CBH1) gene was identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16(6): 276-277) was used to predict isoelectric points and molecular weights.

Cloning of the Penicillium swiecickii GH7 CBH1 Gene from Genomic DNA

The above identified Penicillium swiecickii GH7 CBH1 gene was cloned to the expression vector pCaHj505 (EP2748189B1) and heterologously expressed in Aspergillus oryzae strain MT3568

| | DNA sequence | Protein sequence |
|---|---|---|
| Penicillium swiecickii | SEQ ID NO: 30 | SEQ ID NO: 3 |
| GH7 CBH1 | | |

Based on the DNA information obtained from genome sequencing, oligonucleotide primers, shown below, were designed to amplify the coding sequences of the Penicillium swiecickii GH7 CBH1 gene from the genomic DNA of NN053878. The primers were synthesized by Invitrogen, Beijing, China.

Forward Primer:
(SEQ ID NO: 33)
5′-ACACAACTGGGGATCCACCatggcctccactctttc-3′
Reverse Primer:
(SEQ ID NO: 34)
5′-CCCTCTAGATCTCGAGcgccacatggtcataacatcaag-3′

Lowercase characters of forward primers represented the coding regions of the genes while lowercase characters of reverse primers represented the complementary downstream flanking regions of the coding sequences of both genes. Bold characters represented a region homologous to insertion sites of pCaHj505. The four letters underlined in the forward primers represented the Kozark sequence as the initiation of translation process.

For the amplification of the Penicillium swiecickii GH7 CBH1 gene, 10 pmol of the forward and reverse primers (SEQ ID NOs: 33 and 34) were used in a PCR reaction composed of 2 μl of genomic DNA of NN053878, 10 μl of 5× Phusion® GC Buffer (Finnzymes Oy, Espoo, Finland), 1.5 μl of DMSO, 1 μl of 2.5 mM each of dATP, dTTP, dGTP, and dCTP, and 0.6 unit of PHUSION™ High-Fidelity DNA Polymerase (Finnzymes Oy, Espoo, Finland) and double distilled water to a final volume of 50 μl. The amplification was performed using a Peltier Thermal Cycler (MJ Research Inc., South San Francisco, CA, USA) programmed for denaturing at 94° C. for 3 minutes; 10 cycles of each denaturing at 94° C. for 40 seconds, annealing at 69° C. for 30 seconds, with a 1° C. decrease per cycle and elongation at 72° C. for 2 minutes; then 25 cycles of each at 94° C. for 40 seconds, 59° C. for 40 seconds, and 72° C. for 2 minutes; and a final extension at 72° C. for 7 minutes.

The PCR products were isolated by 1.0% agarose gel electrophoresis using 90 mM Tris-borate and 1 mM EDTA (TBE) buffer where a single product band of ˜1.7 kb for the Penicillium swiecickii GH7 CBH1 gene were visualized under UV light. The PCR product was then purified from solutions by using the Illustra™ GFX™ PCR DNA and Gel Band Purification Kit (GE Healthcare, Buckinghamshire, UK) according to the manufacturers' instructions.

The plasmid of pCaHj505 was digested with BamHI and XhoI. The linearized vector product was isolated by 1.0% agarose gel electrophoresis using TBE buffer and purified using an ILLUSTRA™ GFX™ PCR DNA and Gel Band Purification Kit.

For cloning, the In-Fusion® HD Cloning Kit (Clontech Laboratories, Inc., Mountain View, CA, USA) was used. Briefly, 0.8 μl of linearized pCaHj505, 3.2 μl of the Penicillium swiecickii GH7 CBH1 PCR product were added to 1 μl of 5× In-Fusion mix. Then the ligation reaction solution was incubated at 50° C. for 15 minutes, and kept on ice till E. coli transformation.

The ligation reaction solution was used to transform E. coli TOP10 competent cells (TIANGEN Biotech Co. Ltd., Beijing, China) following the manufacturers' instructions. 20-30 transformants were obtained. Several E. coli transformants were selected and sent for sequencing using 3730XL DNA Analyzers (Applied Biosystems Inc, Foster City, CA, USA) in SinoGenoMax (SinoGenoMax Company Limited, Beijing, China). The one that was confirmed with the right gene insertion and the correct sequence was selected for plasmid DNA preparation. The selected transformants were inoculated to LBA medium and grown at 37° C., 200 rpm, overnight. Plasmid DNA was prepared using a QIAPREP® Spin Miniprep Kit (QIAGEN GmbH, Hilden, Germany). This resulted in plasmid p505-GH7_Pesw, in which transcription of the coding sequence of the Penicillium swiecickii GH7 CBH1 gene was under the control of a TAKA-amylase promoter derived from Aspergillus oryzae.

Expression of the Penicillium swiecickii GH7 CBH1 Gene in Aspergillus oryzae

Aspergillus oryzae strain MT3568 was used for the heterologous expression of the gene encoding the Penicillium swiecickii GH7 CBH1.

Protoplasts of were prepared and transformed according to the method of Christensen et al., 1988, Bio/Technology 6:1419-1422.

Plasmid p505-GH7_Pesw was used to transform MT3568. In brief, 3 μg plasmid DNA was added to 100 μl of protoplasts and after incubation at room temperature for 10 minutes, 300 μl of 60% PEG was added and mixed with protoplasts. Then after another 30 minutes incubation at room temperature, protoplasts were resuspended in ˜6 ml of top agar medium and spread onto the Minimal medium plate. The plate was incubated at 37° C. until transformants were visible and started to sporulate.

The transformation yielded about 20 transformants. Four transformants were isolated on COVE medium reisolation plates and were then inoculated separately into 3 ml of YPM medium in 24-well deep well plate and incubated at 30° C., 150 rpm. After 3 days incubation, 20 μl of supernatant from the culture was analyzed on NUPAGE® NOVEX® 4-12% Bis-Tris Gel with 50 mM MES (Invitrogen Corporation, Carlsbad, CA, USA) according to the manufacturer's instructions. The resulting gel was stained with INSTANTBLUE™ (Expedeon Ltd., Babraham Cambridge, UK). SDS-PAGE profiles of the cultures showed that the majority of the transformants had a major band of approximately ˜56 kDa for the Penicillium swiecickii GH7 CBH1. The expression strain was then designated O62JNC.

Fermentation of Aspergillus oryzae Expression Strains O62JNC

Two fully sporulated slants of the expression strain O62JNC were each washed with 5 ml of DAP4C and inoculated into 14 2-liter flasks each containing 400 ml of DAP4C medium, shaking at 30° C., 80 rpm. The culture broths were harvested on day 3 and filtered by using a 1000 ml Rapid-Flow Bottle Top Filter 0.2 μm aPES membrane (ThermoFisher Scientific, Waltham, Massachusetts, USA). The filtered broth sample was purified as described below.

Purification of Recombinant Penicillium swiecickii CBHI from Aspergillus oryzae O62JNC

A 5600 ml volume of filtered supernatant of Aspergillus oryzae O62JNC was precipitated with ammonium sulfate (80% saturation), the protein was re-dissolved in ddH₂O, followed by adjusting conductivity to 145 ms/cm with (NH₄)₂SO₄, and filtered through a 0.45 μm filter. The final volume was 130 ml. The solution was applied to a 40 ml Phenyl Sepharose high performance column (GE Healthcare, Buckinghamshire, UK), protein was washed with a linear 1.2-0 M (NH₄)₂SO₄ gradient. Fractions were analyzed by SDS-PAGE using a NUPAGE® NOVEX® 4-12% Bis-Tris Gel with 50 mM MES. The resulting gel was stained with INSTANTBLUE™. Fractions containing a band at approximately 56 kDa were pooled. Then the pooled solution was concentrated by ultrafiltration.

### Example 2: Cloning and Identification of Talaromyces verruculosus CBH2

### Genomic DNA Extraction

The Talaromyces verruculosus strain NN046799 was inoculated onto a PDA plate and incubated for 7 days at 37° C. in the darkness. Several mycelia-PDA plugs were inoculated into 500 ml shake flasks containing 100 ml of YPG medium. The flasks were incubated for 4 days at 37° C. with shaking at 160 rpm.

The mycelia were collected by filtration through MIRACLOTH® and frozen under liquid nitrogen. Frozen mycelia were ground, by a mortar and a pestle, to a fine powder, and genomic DNA was isolated using DNeasy® Plant Maxi Kit (24) (QIAGEN GmbH, Hilden, Germany) following the manufacturer's instruction.

### Genome Sequencing, Assembly and Annotation

The extracted genomic DNA sample of NN046799 was delivered to Novozymes A/S for genome sequencing using an ILLUMINA® MiSeq System (Illumina, Inc., San Diego, CA, USA). The raw reads were assembled at Novozymes Denmark using program Spades (Anton Bankevich et al., 2012, Journal of Computational Biology, 19(5): 455-477). The assembled sequences were analyzed using standard bioinformatics methods for gene identification and function prediction. GeneMark-ES fungal version was used for gene prediction. Blastall version 2.2.10 (Altschul et al., 1990, Journal of Molecular Biology, 215(3): 403-410) and HMMER version 2.1.1 were used to predict function based on structural homology. The Talaromyces verruculosus GH6 cellobiohydrolase II (CBH2) gene was identified directly by analysis of the Blast results. The Agene program and SignalP program were used to identify start codons. SignalP program was further used to predict signal peptides. Pepstats was used to predict isoelectric points and molecular weights.

Cloning of the Talaromyces verruculosus GH6 CBH2 Gene from Genomic DNA

The above identified Talaromyces verruculosus GH6 CBH2 gene was cloned to the expression vector pCaHj505 (EP2748189B1) and heterologously expressed in Aspergillus oryzae strain MT3568.

| | DNA sequence | Protein sequence |
|---|---|---|
| Talaromyces verruculosus | SEQ ID NO: 23 | SEQ ID NO: 4 |
| GH6 CBH2 | | |

Based on the DNA information obtained from genome sequencing, oligonucleotide primers, shown below, were designed to amplify the coding sequences of the Talaromyces verruculosus GH6 CBH2 gene from NN046799. The primers were synthesized by Invitrogen, Beijing, China.

Forward Primer:
(SEQ ID NO: 35)
5′-ACACAACTGGGGATCCACCatgctgcgatatctttccattgtt-3′
Reverse Primer:
(SEQ ID NO: 36)
5′-CCCTCTAGATCTCGAGaaaggatattggtgcagtcaaagctg-3′

Lowercase characters of forward primers represented the coding regions of the genes while lowercase characters of reverse primers represented the complementary downstream flanking regions of the coding sequences of both genes. Bold characters represented a region homologous to insertion sites of pCaHj505. The 4 letters underlined in the forward primers represented the Kozark sequence as the initiation of translation process.

For the amplification of the Talaromyces verruculosus GH6 CBH2 gene, 10 pmol of the forward and reverse primers (SEQ ID NO:35 & 36) were used in a PCR reaction composed of 2 μl of genomic DNA of NN046799, 10 μl of 5× Phusion® GC Buffer, 1.5 μl of DMSO, 1.5 μl of 2.5 mM each of dATP, dTTP, dGTP, and dCTP, and 0.6 unit of PHUSION™ High-Fidelity DNA Polymerase and ddH2O to a final volume of 50 μl. The amplification was performed using a Peltier Thermal programmed for denaturing at 98° C. for 1 minute; 10 cycles of each denaturing at 98° C. for 30 seconds, annealing at 70° C. for 30 seconds, with a 1° C. decrease per cycle and elongation at 72° C. for 2 minutes and 30 seconds; then 25 cycles each at 98° C. for 30 seconds, 60° C. for 30 seconds, and 72° C. for 2 minutes and 30 seconds; and a final extension at 72° C. for 7 minutes.

The PCR products were isolated by 1.0% agarose gel electrophoresis using 90 mM Tris-borate and 1 mM EDTA (TBE) buffer where a single product band of ˜1.8 kb band for the Talaromyces verruculosus GH6 CBH2 gene was visualized under UV light. The PCR product was then purified from solutions by using the Illustra™ GFX™ PCR DNA and Gel Band Purification Kit (GE Healthcare, Buckinghamshire, UK) according to the manufacturers' instructions.

The plasmid of pCaHj505 was digested with BamHI and XhoI. The linearized vector product was isolated by 1.0% agarose gel electrophoresis using TBE buffer and purified using an ILLUSTRA™ GFX™ PCR DNA and Gel Band Purification Kit.

For cloning, the In-Fusion® HD Cloning Kit (Clontech Laboratories, Inc., Mountain View, CA, USA) was used. Briefly, 1 μl of linearized pCaHj505, 3 μl of the Talaromyces verruculosus GH6 CBH2 PCR product were added to 1 μl of the 5× In-fusion mix. Then the ligation reaction solution was incubated at 50° C. for 15 minutes, and kept on ice till E. coli transformation.

The ligation reaction solution was used to transform E. coli TOP10 competent cells (TIANGEN Biotech Co. Ltd., Beijing, China) following the manufacturers' instructions. 20-30 transformants were obtained in each transformation. Several E. coli transformants from each transformation were selected and sent for sequencing using 3730XL DNA Analyzers (Applied Biosystems Inc, Foster City, CA, USA) in SinoGenoMax (SinoGenoMax Company Limited, Beijing, China). The one that was confirmed with the right gene insertion and the correct sequence was selected for plasmid DNA preparation. The selected transformants were inoculated to LBA medium and grown at 37° C., 200 rpm, overnight. Plasmid DNA was prepared using a QIAPREP® Spin Miniprep Kit (QIAGEN GmbH, Hilden, Germany). This resulted in plasmid p505-GH6_Tave, in which the transcriptions of the coding sequences of the Talaromyces verruculosus GH6 CBH2 gene was under the control of a TAKA-amylase promoter derived from Aspergillus oryzae.

Expression of the Talaromyces verruculosus GH6 CBH2 Gene in Aspergillus oryzae

Aspergillus oryzae strain MT3568 was used for the heterologous expression of the gene encoding the Talaromyces verruculosus GH6 CBH2.

Protoplasts were prepared and transformed according to the method of Christensen et al., 1988, Bio/Technology 6:1419-1422.

Plasmid p505-GH6_Tave was used to transform MT3568. In brief, 3 μg plasmid DNA was added to 100 μl of protoplasts and after incubation at room temperature for 10 minutes, 300 μl of 60% PEG was added and mixed with protoplasts. Then after another 30 minutes incubation at room temperature, protoplasts were resuspended in ˜6 ml of top agar medium and spread onto the Minimal medium plate. The plate was incubated at 37° C. until transformants were visible and started to sporulate.

The transformation yielded about 20 transformants. Four transformants were isolated on COVE medium reisolation plates and were then inoculated separately into 3 ml of YPM medium in 24-well deep well plate and incubated at 30° C., 150 rpm. After 3 days incubation, 20 μl of supernatant from the culture was analyzed on NUPAGE® NOVEX® 4-12% Bis-Tris Gel with 50 mM MES (Invitrogen Corporation, Carlsbad, CA, USA) according to the manufacturer's instructions. The resulting gel was stained with INSTANTBLUE™ (Expedeon Ltd., Babraham Cambridge, UK). SDS-PAGE profiles of the cultures showed that the majority of the transformants had a major band of approximately ˜65 kDa for the Talaromyces verruculosus GH6 CBH2. The expression strain was then designated as O13UA4 accordingly.

Fermentation of Aspergillus oryzae Expression Strain O13UA4

Five fully sporulated slants of the expression strain O13UA4 was each washed with 5 ml of DAP4C and inoculated into 28 2-liter flasks each containing 400 ml of DAP4C medium, shaking at 30° C., 80 rpm. The culture was harvested on day 3 and filtered by using a 0.2 μm aPES Membrane. The filtered broth sample was purified as described below.

Purification of Recombinant Talaromyces verruculosus CBHII from Aspergillus oryzae O13UA4

A 11200 ml volume of filtered supernatant of Aspergillus oryzae O13UA4 was precipitated with ammonium sulfate (80% saturation), the protein was re-dissolved in ddH₂O, followed by adjusting conductivity to 145 ms/cm with (NH₄)₂SO₄, and filtered through a 0.45 μm filter. The final volume was 130 ml. The solution was applied to a 40 ml Phenyl Sepharose high performance column, proteins were washed with a linear 1.2-0 M (NH₄)₂SO₄ gradient. Fractions were analyzed by SDS-PAGE using a NUPAGE® NOVEX® 4-12% Bis-Tris Gel with 50 mM MES. The resulting gel was stained with INSTANTBLUE™. Fractions containing a band at approximately 65 kDa were pooled. Then the pooled solution was concentrated by ultrafiltration.

### Example 3: Cloning and Identification of Cladosporium antarcticum EG2

### Genomic DNA Extraction

The fungal strain NN058608 was inoculated onto a PDA plate and incubated for 7 days at 25° C. in the darkness. Several mycelia-PDA plugs were inoculated into 500 ml shake flasks containing 100 ml of YPG medium. The flasks were incubated for 4 days at 25° C. with shaking at 160 rpm. The mycelia were collected by filtration through MIRACLOTH® (Calbiochem, La Jolla, CA, USA) and frozen under liquid nitrogen. Frozen mycelia were ground, by a mortar and a pestle, to a fine powder, and genomic DNA was isolated using MP Fast DNA spin kit for soil (MP Biomedicals, Santa Ana, California, USA) following the manufacturer's instruction.

### Genome Sequencing, Assembly and Annotation

The extracted genomic DNA sample of NN058608 was delivered to Exiqon A/S (Denmark) for genome sequencing using an ILLUMINA® MiSeq System (Illumina, Inc., San Diego, CA, USA). The raw reads were assembled at Novozymes Denmark using program Spades (Anton Bankevich et al., 2012, Journal of Computational Biology, 19(5): 455-477). The assembled sequences were analyzed using standard bioinformatics methods for gene identification and function prediction. GeneMark-ES fungal version (Ter-Hovhannisyan V et al., 2008, Genome Research 18(12): 1979-1990) was used for gene prediction. Blastall version 2.2.10 (Altschul et al., 1990, Journal of Molecular Biology, 215(3): 403-410) and HMMER version 2.1.1 (National Center for Biotechnology Information (NCBI), Bethesda, MD, USA) were used to predict function based on structural homology. The Cladosporium antarcticum family GH5 endo-beta-1,4-glucanase (SEQ ID NO: 31 for the DNA sequence and SEQ ID NO: 5 for the deduced amino acid sequence) was identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16(6): 276-277) was used to predict isoelectric points and molecular weights.

Cloning of the Cladosporium antarcticum GH5 Endo-Beta-1,4-Glucanase Gene from Genomic DNA

The above identified Cladosporium antarcticum GH5 endo-beta-1,4-glucanase gene was cloned to expression vectors and recombinantly expressed in Aspergillus oryzae. Details were listed in Table 2 below.

TABLE 2

| | DNA | Protein | | Host |
|---|---|---|---|---|
| | sequence | sequence | Vector | strain |
| Cladosporium antarcticum | SEQ ID | SEQ ID | pDAU724 | Dau785 |
| GH5 endo-beta-1,4-glucanase | NO: 31 | NO: 5 | | |

The expression vector pDAU724 was described in WO2016/026938.

Based on the DNA information obtained from genome sequencing, oligonucleotide primers, shown below, were designed to amplify the coding sequence of the Cladosporium antarcticum GH5 endo-beta-1,4-glucanase from the genomic DNA of NN058608.

Forward Primer:
(SEQ ID NO: 37)
5′-ACACAACTGGGGATCCACCatgaagtcttctttcatcgctgttgc
a-3′
Reverse Primer:
(SEQ ID NO: 38)
5′-GTCACCCTCTAGATCTcgagcgactggactacaaatccagcaaa
gatc-3′

Lowercase characters of forward primers represent the coding regions of the genes while lowercase characters of reverse primers represent the downstream flanking regions of the coding sequence. Bold characters represent a region homologous to insertion sites of pPFJO355 or pDAU724. The 4 letters underlined in the forward primers represent the Kozark sequence as the initiation of translation process.

For the amplification of the Cladosporium antarcticum GH5 endo-beta-1,4-glucanase gene, 10 pmol of the forward and reverse primers (SEQ ID NO: 37 & 38) were used in a PCR reaction composed of 2 μl of genomic DNA of Cladosporium antarcticum strain NN058608, 10 μl of 5× Phusion® GC Buffer (Finnzymes Oy, Espoo, Finland), 1 μl of 2.5 mM each of dATP, dTTP, dGTP, and dCTP, and 0.6 unit of PHUSION™ High-Fidelity DNA Polymerase (Finnzymes Oy, Espoo, Finland) and double distilled water to a final volume of 50 μl. The amplification was performed using a Peltier Thermal Cycler (MJ Research Inc., South San Francisco, CA, USA) programmed for denaturing at 98° C. for 1 minute; 10 cycles of denaturing each at 98° C. for 30 seconds, annealing at 68° C. for 30 seconds, with a 1° C. decrease per cycle and elongation at 72° C. for 1 minute; then 26 cycles each at 98° C. for 30 seconds, 58° C. for 30 seconds, and 72° C. for 1 minute; and a final extension at 72° C. for 10 minutes.

The PCR product was isolated by 1.0% agarose gel electrophoresis using 90 mM Tris-borate and 1 mM EDTA (TBE) buffer where a single product band of approximately 1.4 kb for Cladosporium antarcticum GH5 endo-beta-1,4-glucanase gene was visualized under UV light. The PCR product was then purified from solution by using the Illustra™ GFX™ PCR DNA and Gel Band Purification Kit (GE Healthcare, Buckinghamshire, UK) according to the manufacturers' instructions.

The expression vector was linearized by restriction digestion. The plasmid of pDAU724 was digested with BamHI and XhoI. The linearized vector product was isolated by 1.0% agarose gel electrophoresis using TBE buffer and purified using an ILLUSTRA™ GFX™ PCR DNA and Gel Band Purification Kit.

For cloning of the Cladosporium antarcticum GH5 endo-beta-1,4-glucanase gene, the In-Fusion® HD Cloning Kit (Clontech Laboratories, Inc., Mountain View, CA, USA) was used. Briefly, 0.3 μl of linearized pDAU724 vector and 3.7 μl of GH5 endo-beta-1,4-glucanase PCR product were added to 1 μl of the 5× in-fusion mix. Then the ligation reaction solution was incubated at 50° C. for 15 minutes. It was kept on ice till E. coli transformation.

The ligation reaction solution was used to transform E. coli TOP10 competent cells (TIANGEN Biotech Co. Ltd., Beijing, China) following the manufacturers' instructions. 20-30 transformants were obtained in each transformation. Two E. coli transformants from the transformation were selected and sent for sequencing using 3730XL DNA Analyzers (Applied Biosystems Inc, Foster City, CA, USA) in SinoGenoMax (SinoGenoMax Company Limited, Beijing, China). The one that was confirmed with the right gene insertion and the correct sequence was selected for plasmid DNA preparation. The selected transformants were inoculated to LBA medium and grown at 37° C., 200 rpm, overnight. Plasmid DNA was prepared using a QIAPREP® Spin Miniprep Kit (QIAGEN GmbH, Hilden, Germany). This resulted in plasmids pDau724-GH5_Clan1, in which the transcription of the Cladosporium antarcticum GH5 endo-beta-1,4-glucanase gene coding sequence was under the control of a NA2TPI promoter.

Expression of Cladosporium antarcticum GH5 Endo-Beta-1,4-Glucanase Gene in Aspergillus oryzae

Aspergillus oryzae strain DAu785 was used for the heterologous expression of the gene encoding the Cladosporium antarcticum GH5 endo-beta-1,4-glucanase. Protoplasts were prepared and transformed according to the method of Christensen et al., 1988, Bio/Technology 6:1419-1422.

Plasmids pDau724-GH5_Clan1 was used to transform Dau785. In brief, 3 μg DNA was added to 100 μl of protoplasts and after incubation at room temperature for 10 minutes, 300 μl of 60% PEG was added and mixed with protoplasts. After another 30 minutes incubation at room temperature, protoplasts were resuspended in ˜6 ml of top agar medium and spread onto the Minimal medium plate. The plate was incubated at 37° C. until transformants were visible and started to sporulate.

The transformations yielded about 20 transformants. Four transformants were isolated on COVE medium reisolation plates and were then inoculated separately into 3 ml of YPM medium in 24-well deep well plate and incubated at 30° C., 150 rpm. After 3 days incubation, 20 μl of supernatant from each culture were analyzed on NUPAGE® NOVEX® 4-12% Bis-Tris Gel with 50 mM MES (Invitrogen Corporation, Carlsbad, CA, USA) according to the manufacturer's instructions. The resulting gel was stained with INSTANTBLUE™ (Expedeon Ltd., Babraham Cambridge, UK). SDS-PAGE profiles of the cultures showed that the majority of the transformants had a major band of approximately 43 kDa for the Cladosporium antarcticum GH5 endo-beta-1,4-glucanase. The resulting expression strain was designated as O54U3N.

Fermentation of Aspergillus oryzae Expression Strain O54U3N

Two fully sporulated slants of the expression strain O54U3N were washed with 10 ml of DAP4C and inoculated into 10 2-liter flasks each containing 400 ml of DAP4C medium, shaking at 30° C., 80 rpm. The culture broth was harvested by using a 1000 ml Rapid-Flow Bottle Top Filter 0.2 μm aPES membrane (ThermoFisher Scientific, Waltham, Massachusetts, USA). The filtered broth sample was purified as described below.

Purification of Recombinant Cladosporium antarcticum GH5 Endo-Beta-1,4-Glucanase from Aspergillus oryzae O54U3N

4000 ml supernatant of the recombinant strain O54U3N was precipitated with ammonium sulfate (80% saturation), the protein was re-dissolved in ddH2O, followed by adjusting conductivity to 145 ms/cm with (NH₄)₂SO₄, and filtered through a 0.45 μm filter. The final volume was 90 ml. The solution was applied to a 40 ml Phenyl hydrophobic column (GE Healthcare, Buckinghamshire, UK), proteins were washed with a linear 1.8-0.0 M (NH₄)₂SO₄ gradient. Fractions were analyzed by SDS-PAGE using a NUPAGE® NOVEX® 4-12% Bis-Tris Gel with 50 mM MES. The resulting gel was stained with INSTANTBLUE™. Fractions containing a band at approximately 43 kDa were pooled. The pooled solution was adjusted conductivity to 145 ms/cm with (NH₄)₂SO₄ and applied to a 20 ml Phenyl Sepharose High Performance column (GE Healthcare, Buckinghamshire, UK), proteins were washed with a linear 1.5-0.0 M (NH₄)₂SO₄ gradient. Fractions were analyzed by SDS-PAGE using a NUPAGE® NOVEX® 4-12% Bis-Tris Gel with 50 mM MES. The resulting gel was stained with INSTANTBLUE™. Fractions containing a band at approximately 43 kDa were pooled. Then the pooled solution was concentrated by ultrafiltration.

### Example 4: Cloning and Identification of Talaromyces pinophilus GH3 Beta-Glucosidase

### Genomic DNA Extraction

The fungal strain NN046877 was inoculated onto a PDA plate and incubated for 7 days at 37° C. in the darkness. Several mycelia-PDA plugs were inoculated into 500 ml shake flasks containing 100 ml of YPG medium. The flasks were incubated for 3 days at 37° C. with shaking at 160 rpm. The mycelia were collected by filtration through MIRACLOTH® and frozen under liquid nitrogen. Frozen mycelia were ground, by a mortar and a pestle, to a fine powder, and genomic DNA was isolated using DNeasy® Plant Maxi Kit (24) (QIAGEN GmbH, Hilden, Germany) following the manufacturer's instruction.

### Genome Sequencing, Assembly and Annotation

The extracted genomic DNA sample of NN046877 was delivered to Beijing Genome Institute (BGI, Shenzhen, China) for genome sequencing using an ILLUMINA® GA2 System (Illumina, Inc., San Diego, CA, USA). The raw reads were assembled at BGI using program SOAPdenovo (Li et al., 2010, Genome Research, 20:265-72). The assembled sequences were analyzed using standard bioinformatics methods for gene identification and functional prediction. GeneMark-ES fungal version (Ter-Hovhannisyan V et al., 2008, Genome Research, 18(12): 1979-1990) was used for gene prediction. Blastall version 2.2.25 (Altschul et al., 1990, Journal of Molecular Biology, 215(3): 403-410) and HMMER version 2.1.1 (National Center for Biotechnology Information (NCBI), Bethesda, MD, USA) were used to predict function based on structural homology. The Talaromyces pinophilus family GH3 beta-glucosidase (SEQ ID NO: 32 for the DNA sequence and SEQ ID NO: 6 for the deduced amino acid sequence) was identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16(6): 276-277) was used to predict isoelectric points and molecular weights.

Cloning of the Cladosporium antarcticum GH5 Endo-Beta-1,4-Glucanase and the Talaromyces pinophilus GH3 Beta-Glucosidase Genes from Genomic DNA

The above identified Talaromyces pinophilus GH3 beta-glucosidase gene was cloned to expression vectors and recombinantly expressed in Aspergillus oryzae. Details were listed in Table 3 below.

TABLE 3

| | | | |
|---|---|---|---|
| DNA | Protein | | Host |
| sequence | sequence | Vector | strain |

| | | | | |
|---|---|---|---|---|
| Talaromyces pinophilus | SEQ ID | SEQ ID | pPFJO355 | HowB101 |
| GH3 beta-glucosidase | NO: 32 | NO: 6 | | |

The expression vector pPFJO355 was described in WO 2011/005867.

Based on the DNA information obtained from genome sequencing, oligonucleotide primers, shown below, were designed to amplify the coding sequence of the Talaromyces pinophilus GH3 beta-glucosidase from NN046877.

Forward Primer:
(SEQ ID NO: 39)
5′-ACACAACTGGGGATCCACCatgcggaacagtttattgatttcg-3′
Reverse Primer:
(SEQ ID NO: 40)
5′-GTCACCCTCTAGATCTacaaaagggtggaagccgatc-3′

Lowercase characters of forward primers represent the coding regions of the genes while lowercase characters of reverse primers represent the downstream flanking regions of the coding sequence. Bold characters represent a region homologous to insertion sites of pPFJO355. The four letters underlined in the forward primers represent the Kozark sequence as the initiation of translation process.

For the amplification of the Talaromyces pinophilus GH3 beta-glucosidase gene, 20 pmol of the forward and reverse primers (SEQ ID NO: 39 & 40) were used in a PCR reaction composed of 2 μl of genomic DNA of Talaromyces pinophilus strain NN046877, 10 μl of 5× Phusion® GC Buffer, 1.5 μl of DMSO, 1.5 μl of 2.5 mM each of dATP, dTTP, dGTP, and dCTP, and 0.6 unit of PHUSION™ High-Fidelity DNA Polymerase and ddH₂O to a final volume of 50 μl. The amplification was performed using a Peltier Thermal programmed for denaturing at 98° C. for 1 minute; 6 cycles of denaturing each at 98° C. for 15 seconds, annealing at 66° C. for 30 seconds, with a 1° C. decrease per cycle and elongation at 72° C. for 1 minute and 45 seconds; then 25 cycles each at 98° C. for 15 seconds, 62° C. for 30 seconds, and 72° C. for 1 minute and 45 seconds; and a final extension at 72° C. for 5 minutes.

The PCR product was isolated by 1.0% agarose gel electrophoresis using 90 mM Tris-borate and 1 mM EDTA (TBE) buffer where a single product band of approximately 3.0 kb band for Talaromyces pinophilus GH3 beta-glucosidase gene were visualized under UV light. The PCR product was then purified from solution by using the Illustra™ GFX™ PCR DNA and Montage™ PCR96 Clean-Up (Millipore Corporation, Billerica, MA 01821, USA) according to the manufacturers' instructions.

The expression vector was linearized by restriction digestion. The plasmid of pPFJO355 was digested with BamHI and BgIII. The linearized vector product was isolated by 1.0% agarose gel electrophoresis using TBE buffer and purified using an ILLUSTRA™ GFX™ PCR DNA and Gel Band Purification Kit.

For cloning of the Talaromyces pinophilus GH3 beta-glucosidase gene, the In-Fusion 2.0 CF dry down Cloning kit (Clontech Laboratories, Inc., Mountain View, CA, USA) was used. The In-Fusion Dry Down pellet was suspended in 3 μl of ddH₂O and 1 μl of the linearized pPFJO355 vector. Then 2 μl of the suspension solution was added to 3 μl of GH3 beta-glucosidase gene PCR product. The ligation reaction solution was incubated at 37° C. for 15 minutes followed by 50° C. for 15 minutes. It was kept at room temperature till the E. coli transformation.

The ligation reaction solution was used to transform E. coli TOP10 competent cells (TIANGEN Biotech Co. Ltd., Beijing, China) following the manufacturers' instructions. 20-30 transformants were obtained in each transformation. Two E. coli transformants were selected and sent for sequencing using 3730XL DNA Analyzers (Applied Biosystems Inc, Foster City, CA, USA) in SinoGenoMax (SinoGenoMax Company Limited, Beijing, China). The one that was confirmed with the right gene insertion and the correct sequence was selected for plasmid DNA preparation. The selected transformants were inoculated to LBA medium and grown at 37° C., 200 rpm, overnight. Plasmid DNA was prepared using a QIAPREP® Spin Miniprep Kit (QIAGEN GmbH, Hilden, Germany). This resulted in plasmid pGH3_ZY503522_84, in which the transcription of the Talaromyces pinophilus GH3 beta-glucosidase gene coding sequence was under the control of an Aspergillus oryzae alpha-amylase gene promoter.

Expression of Talaromyces pinophilus GH3 Beta-Glucosidase Gene in Aspergillus oryzae

Aspergillus oryzae strain HowB101 was used for the heterologous expression of the gene encoding the Talaromyces pinophilus GH3 beta-glucosidase. Protoplasts were prepared and transformed according to the method of Christensen et al., 1988, Bio/Technology 6:1419-1422.

Plasmids pGH3_ZY503522_84 was used to transform HowB101. In brief, 3 μg DNA was added to 100 μl of protoplasts and after incubation at room temperature for 10 minutes, 300 μl of 60% PEG was added and mixed with protoplasts. After another 30 minutes incubation at room temperature, protoplasts were resuspended in ˜6 ml of top agar medium and spread onto the Minimal medium plate. The plate was incubated at 37° C. until transformants were visible and started to sporulate.

The transformations yielded about 20 transformants each. Four transformants were isolated on COVE medium reisolation plates and were then inoculated separately into 3 ml of YPM medium in 24-well deep well plate and incubated at 30° C., 150 rpm. After 3 days incubation, 20 μl of supernatant from each culture were analyzed on NUPAGE® NOVEX® 4-12% Bis-Tris Gel with 50 mM MES (Invitrogen Corporation, Carlsbad, CA, USA) according to the manufacturer's instructions. The resulting gel was stained with INSTANTBLUE™ (Expedeon Ltd., Babraham Cambridge, UK). SDS-PAGE profiles of the cultures showed that the majority of the transformants had a major band of approximately 90 kDa for the Talaromyces pinophilus GH3 beta-glucosidase. The resulting expression strain was designated O3XUX.

Fermentation of Aspergillus oryzae Expression Strain O3XUX

One fully sporulated slant of the expression strain O3XUX was washed with 10 ml of YPM and inoculated into 4 2-liter flasks each containing 400 ml of YPM medium, shaking at 30° C., 80 rpm. The culture was harvested on day 3 and filtered using a 0.45 μm DURAPORE Membrane (Millipore, Bedford, MA, USA). The filtered broth sample was purified as described below.

Purification of Recombinant Penicillium pinophilum GH3 Beta-Glucosidase from Aspergillus oryzae O3XUX

1600 ml supernatant of the recombinant strain O3XUX was precipitated with ammonium sulfate (80% saturation) and re-dissolved in 80 ml of 20 mM Tris-HCl buffer, pH7.0, then dialyzed against the same buffer and filtered through a 0.45 mm filter, the final volume was 95 ml. The solution was applied to a 40 ml Q FF column (GE) equilibrated in 20 mM Tris-HCl buffer, pH7.0, and the proteins was eluted with a linear NaCl gradient (0-0.4M). Fractions eluted with 0.1-0.3M NaCl were collected. The collected sample was dialyzed against 20 mM Tris-HCl buffer, pH7.0, and applied to the same column again, the proteins was eluted with a linear NaCl gradient (0.1-0.3M). Fractions were evaluated by SDS-PAGE (NP0336BOX, NUPAGE 4-12% BT GEL 1.5 MM15 W). Fractions containing a band of approximately 90 kDa were pooled. Then the pooled solution was concentrated by ultrafiltration.

### Example 5: Construction of Yeast Strains Expressing a Talaromyces pinophilus GH3 Beta-Glucosidase

This example describes the construction of yeast cells expressing the Talaromyces pinophilus GH3 beta-glucosidase of SEQ ID NO: 6 under the control of one S. cerevisiae promoter: pTDH3, which is a strong constitutive promoter. Three pieces of DNA containing promoters, genes and terminators were designed to allow for homologous recombination between the 3 DNA fragments and into the XII-2 locus of the yeast MeJi797. The resulting strain would have one 5′ homology containing fragment with a promoter, 1 promoter and gene containing fragment, and one 3′ homology fragment with a terminator integrated into the S. cerevisiae genome at the XII-2 locus.

Construction of the 5′ XII-2 Homology Containing Fragment with pTDH3 (Left Fragment 1)

The linear DNA containing 500 bp homology to the XII-2 site and the S. cerevisiae pTDH3 promoter was PCR amplified from HP30 plasmid DNA with primers 1230183 (5′-TCTTT TCGCG CCCTG GAAA-3′; SEQ ID NO: 41) and 1230194 (5′-TTTGT TTGTT TATGT GTGTT TATTC GAAAC TAAGT TC-3′; SEQ ID NO: 42). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Construction of the 3′ XII-2 Homology Containing Fragment with tPRM9 (Right Fragment 1)

The linear DNA containing 500 bp homology to the XII-2 site and the S. cerevisiae tPRM9 terminator was PCR amplified from TH12 plasmid DNA with primers 1230177 (5′-ACAGA AGACG GGAGA CACTA GC-3′; SEQ ID NO: 43) and 1230216 (5′-TCAGT CCAAT GACAG TATTT TCTCC TTCTC AC-3′; SEQ ID NO: 44). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

### Integration of the Left, Middle and Right-Hand Fragments

The yeast MeJi797 was transformed with the left, middle and right integration fragments described above. In each transformation pool, the left fragment and right fragment with 50 ng of each fragment was used. There was 1 set of the left and right fragment pools used. There is also an additional middle fragment consisted of a beta-glucosidase gene and a signal peptide with ˜50 ng of each fragment (200 ng total). The synthetic beta-glucosidase DNA fragment contains genes for: P23WFT. To aid homologous recombination of the left, middle and right fragments at the genomic XII-2 sites a plasmid containing MAD7 and guide RNA specific to XII-2 (pMIBa638) was also used in the transformation. The one synthetic middle component was combined with the left and right fragments and transformed into the into S. cerevisiae strain MeJi797 following a yeast electroporation protocol. Transformants were selected on YPD+cloNAT to select for transformants that contain the Mad7 plasmid pMIBa638. Transformants were either picked manually by hand onto YPD plates or by using a Q-pix Colony Picking System (Molecular Devices) to inoculate 1 well of 96-well plate containing YPD media. The plates were grown for 2 days then glycerol was added to 20% final concentration and the plates were stored at −80° C. until needed. Integration of the beta-glucosidase construct was verified by PCR with locus specific primers and subsequent sequencing. The resulting strains generated in MeJi797 in this example are shown in Table 4.

TABLE 4
Strains with beta-glucoside gene P23WFT to MeJi797

| Strain ID | Promoter1 | BG gene |
|---|---|---|
| MeJi797 | | |
| S833-E04 | pTDH3 | P23WFT |
| S833-G04 | pTDH3 | P23WFT |

### Example 6: Construction of Yeast Strains Expressing a Talaromyces verruculosus CBH2

This example describes the construction of yeast cells expressing a Talaromyces verruculosus CBH2 of SEQ ID NO: 4 under the control of one S. cerevisiae promoter: pSeTDH3, which is a strong constitutive promoter. Three pieces of DNA containing promoters, genes and terminators were designed to allow for homologous recombination between the 3 DNA fragments and into the X-3 locus of the yeast S709-A06 (MeJi797 with A. fumigatus beta-glucosidase). The resulting strain would have one 5′ homology containing fragment with a promoter, 1 promoter and gene containing fragment, and one 3′ homology fragment with a terminator integrated into the S. cerevisiae genome at the X-3 locus.

Construction of the 5′ X-3 Homology Containing Fragment with pSeTDH3 (Left Fragment 2)

The linear DNA containing 500 bp homology to the X-3 site and the S. cerevisiae pSeTDH3 promoter was PCR amplified from HP6 plasmid DNA with primers 1230181 (5′-AACGA CAGCA CAAAG GAACT TTCAC-3′; SEQ ID NO: 45) and 1230196 (5′-TTTTA TTGTA TGTGT GTGTG TTTGA AACTA AAGTT CTTG-3′; SEQ ID NO: 46). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Construction of the 3′ X-3 Homology Containing Fragment with tPDC6 (Right Fragment 2)

The linear DNA containing 500 bp homology to the X-3 site and the S. cerevisiae tPDC6 terminator was PCR amplified from TH39 plasmid DNA with primers 1230179 (5′-GCCAT TAGTA GTGTA CTCAA ACGAA TTATT G-3′; SEQ ID NO: 47) and 1230745 (5′-GGCTA CTGAT TTTGT TAAGC AACTC ATCAA G-3′; SEQ ID NO: 48. Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Integration of the Left, Middle and Right-Hand Fragments to Generate Yeast Strains with P43VY6

The yeast S709-A06 (MeJi797 expressing the A. fumigatus beta-glucosidase of SEQ ID NO: 8) was transformed with the left, middle and right integration fragments described above. In each transformation pool, the left fragment and right fragment with 50 ng of each fragment was used. There was 1 set of the left and right fragment pools used. There is also an additional middle fragment consisted of a CBH2 gene and a signal peptide with ˜50 ng of each fragment (200 ng total). The synthetic CBH2 DNA fragment contains genes for: P43VY6. To aid homologous recombination of the left, middle and right fragments at the genomic X-3 sites a plasmid containing MAD7 and guide RNA specific to X-3 (pMIBa647) was also used in the transformation. The one synthetic middle component was combined with the left and right fragments and transformed into the into S. cerevisiae strain S709-A06 following a yeast electroporation protocol. Transformants were selected on YPD+cloNAT to select for transformants that contain the Mad7 plasmid pMIBa647. Transformants were either picked manually by hand onto YPD plates or by using a Q-pix Colony Picking System (Molecular Devices) to inoculate 1 well of 96-well plate containing YPD media. The plates were grown for 2 days then glycerol was added to 20% final concentration and the plates were stored at −80° C. until needed. Integration of the CBH2 construct was verified by PCR with locus specific primers and subsequent sequencing. The strains generated in S709-A06 of this example are were designated S1131-A05 and S1131-B05.

### Example 7: Construction of Yeast Strains Expressing a Penicillium emersonii CBH1 and a Trichoderma reesei Endo-Glucanase

This example describes the construction of yeast cells expressing the Penicillium emersonii CBH1 of SEQ ID NO: 1 under the control of one S. cerevisiae promoter: pSeTDH3, which is a strong constitutive promoter, while also expressing the Trichoderma reesei EG1 of SEQ ID NO: 29 under the control of one S. cerevisiae promoter: pSKTDH3. Five pieces of DNA containing promoters, genes and terminators were designed to allow for homologous recombination between the 5 DNA fragments and into the X-3 locus of the yeast S709-A06 (MeJi797 expressing the A. fumigatus beta-glucosidase of SEQ ID NO: 8). The resulting strain would have one 5′ homology containing fragment with a promoter, 2 promoter and gene containing fragments, 1 terminator and promoter containing fragment, and one 3′ homology fragment with a terminator integrated into the S. cerevisiae genome at the X-3 locus.

Construction of the pSKTDH3 Containing Fragment with tPDC6 (Middle Fragment 1)

The linear DNA containing 143 bp homology to the S. cerevisiae tPDC6 terminator and the pSkTDH3 promoter was PCR amplified from TP46 plasmid DNA with primers 1230179 (5′-GCCAT TAGTA GTGTA CTCAA ACGAA TTATT G-3′; SEQ ID NO: 49) and 1230197 (5′-GTTTA GTTAA TTATA GTTCG TTGAC TGTGT TTCTT G-3′; SEQ ID NO: 50). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Construction of the P33517 Containing Fragment with tENO2 (Middle Fragment 2)

The linear DNA containing 143 bp homology to the S. cerevisiae tENO2 terminator and the pSkTDH3 promoter was PCR amplified from S709-H₀₈ genomic DNA with primers 1230248 (5′-GGTCT CCTTT CTTTC AAGAA ACACA GTC-3′; SEQ ID NO: 51) and 1230232 (5′-ATGAT GAAAA AATAA GCAGA AAAGA CTAAT AATTC TTAG-3′; SEQ ID NO: 52). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Construction of the 3′ X-3 Homology Containing Fragment with tENO2 (Right Fragment 3)

The linear DNA containing 500 bp homology to the X-3 site and the S. cerevisiae tENO2 terminator was PCR amplified from TH36 plasmid DNA with primers 1230176 (5′-AGTGC TTTTA ACTAA GAATT ATTAG TCTTT TCTGC-3′; SEQ ID NO: 53) and 1230745 (5′-GGCTA CTGAT TTTGT TAAGC AACTC ATCAA G-3′; SEQ ID NO: 54). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Integration of the Left, Middle and Right-Hand Fragments to Generate Yeast Strains with CBH1 and EG1

The yeast S709-A06 was transformed with the left, two middle and right integration fragments described above. In each transformation pool, the left fragment and right fragment with 50 ng of each fragment was used. There was 1 set of the left, two middle, and one right fragment pools used: left fragment 2 (supra), middle fragment 1 (supra), middle fragment 2 (supra), and right fragment 3 (supra), designated Blend 1 master mix. There is also an additional middle fragment consisted of a signal peptide and CBH1 gene and a signal peptide with ˜50 ng of each fragment (200 ng total). The synthetic CBH1 DNA fragment contains genes for: P244YG. To aid homologous recombination of the left, two middle and right fragments at the genomic X-3 sites a plasmid containing MAD7 and guide RNA specific to X-3 (pMIBa647) was also used in the transformation. The one synthetic middle component was combined with the Blend 1 master mix and transformed into the into S. cerevisiae strain S709-A06 following a yeast electroporation protocol. Transformants were selected on YPD+cloNAT to select for transformants that contain the Mad7 plasmid pMIBa647. Transformants were either picked manually by hand onto YPD plates or by using a Q-pix Colony Picking System (Molecular Devices) to inoculate 1 well of 96-well plate containing YPD media. The plates were grown for 2 days then glycerol was added to 20% final concentration and the plates were stored at −80° C. until needed. Integration of the CBH1 and EG1 construct was verified by PCR with locus specific primers and subsequent sequencing. The strains generated in S709-A06 in this example resulted in strains.

### Example 8: Construction of Yeast Strains Expressing an Aspergillus fumigatus CBH1 and a Cladosporium antareticum Endo-Glucanase

This example describes the construction of yeast cells expressing an Aspergillus fumigatus CBH1 of SEQ ID NO: 9 under the control of one S. cerevisiae promoter: pSeTDH3, which is a strong constitutive promoter, while also expressing the Cladosporium antareticum EG2 of SEQ ID NO: 5 under the control of one S. cerevisiae promoter: pSKTDH3. Five pieces of DNA containing promoters, genes and terminators were designed to allow for homologous recombination between the 5 DNA fragments and into the X-3 locus of the yeast S709-A06 (MeJi797 expressing the A. fumigatus beta-glucosidase of SEQ ID NO: 8). The resulting strain would have one 5′ homology containing fragment with a promoter, 2 promoter and gene containing fragments, 1 terminator and promoter containing fragment, and one 3′ homology fragment with a terminator integrated into the S. cerevisiae genome at the X-3 locus.

Construction of the P64DAT Containing Fragment with tENO2 (Middle Fragment 3)

The linear DNA containing 143 bp homology to the S. cerevisiae tENO2 terminator and the pSkTDH3 promoter was PCR amplified from P64DAT synthetic TWIST DNA with primers 1230248 (5′-GGTCT CCTTT CTTTC AAGAA ACACA GTC-3′; SEQ ID NO: 55) and 1230232 (5′-ATGAT GAAAA AATAA GCAGA AAAGA CTAAT AATTC TTAG-3′; SEQ ID NO: 56). Fifty pmoles each of forward and reverse primer was used in a PCR reaction containing 5 ng of plasmid DNA as template, 1× Platinum SuperFi HF Buffer (Thermo Fisher Scienctific), and 2 units SuperFi DNA polymerase in a final volume of 50 μL. The PCR was performed in a T100™ Thermal Cycler (Bio-Rad Laboratories, Inc.) programmed for one cycle at 98° C. for 30 seconds followed by 35 cycles each at 98° C. for 10 seconds, 60° C. for 10 seconds, and 72° C. for 1.5 minutes with a final extension at 72° C. for 5 minutes. Following thermocycling, the PCR reaction products gel isolated and cleaned up using the QIAquick Gel Extraction kit (Qiagen).

Integration of the Left, Middle and Right-Hand Fragments to Generate Yeast Strains with CBH1 and EG2

The yeast S709-A06 was transformed with the left, two middle and right integration fragments described above. In each transformation pool, the left fragment and right fragment with 50 ng of each fragment was used. There was 1 set of the left, two middle, and one right fragment pools used: left fragment 2 (Example 6), middle fragment 1 (Example 7), middle fragment 3 (Example 7), and right fragment 3 (supra), designated Blend 2 master mix. There is also an additional middle fragment consisted of a signal peptide and CBH1 gene and a signal peptide with ˜50 ng of each fragment (200 ng total). The synthetic CBH1 DNA fragment contains genes for: P54TVX. To aid homologous recombination of the left, two middle and right fragments at the genomic X-3 sites a plasmid containing MAD7 and guide RNA specific to X-3 (pMIBa647) was also used in the transformation. The one synthetic middle component was combined with the Blend 2 master mix and transformed into the into S. cerevisiae strain S709-A06 following a yeast electroporation protocol. Transformants were selected on YPD+cloNAT to select for transformants that contain the Mad7 plasmid pMIBa647. Transformants were either picked manually by hand onto YPD plates or by using a Q-pix Colony Picking System (Molecular Devices) to inoculate 1 well of 96-well plate containing YPD media. The plates were grown for 2 days then glycerol was added to 20% final concentration and the plates were stored at −80° C. until needed. Integration of the CBH1 and EG2 construct was verified by PCR with locus specific primers and subsequent sequencing.

### Example 9: Construction of Yeast Strains Expressing an Aspergillus fumigatus CBH1 and a Cladosporium antareticum Endo-Glucanase

This example describes the construction of yeast cells containing a CBH2 under the control of one S. cerevisiae promoter: pSeTDH3, which is a strong constitutive promoter. It also contains an EG2 under the control of one S. cerevisiae promoter: pSKTDH3. Five pieces of DNA containing promoters, genes and terminators were designed to allow for homologous recombination between the 5 DNA fragments and into the X-3 locus of the yeast S709-A06 (MeJi797 expressing the A. fumigatus beta-glucosidase of SEQ ID NO: 8). The resulting strain would have one 5′ homology containing fragment with a promoter, 2 promoter and gene containing fragments, 1 terminator and promoter containing fragment, and one 3′ homology fragment with a terminator integrated into the S. cerevisiae genome at the X-3 locus.

Integration of the Left, Middle and Right-Hand Fragments to Generate Yeast Strains with CBH2 and EG2

The yeast S709-A06 was transformed with the left, two middle and right integration fragments described above. In each transformation pool, the left fragment and right fragment with 50 ng of each fragment was used. There was 1 set of the left, two middle, and one right fragment pools used: left fragment 2 (supra), middle fragment 1 (supra), middle fragment 3 (supra), and right fragment 3 (supra), designated Blend 2 master mix. There is also an additional middle fragment consisted of a signal peptide and CBH2 gene and a signal peptide with ˜50 ng of each fragment (200 ng total). The synthetic CBH1 DNA fragment contains genes for: P43VY6. To aid homologous recombination of the left, two middle and right fragments at the genomic X-3 sites a plasmid containing MAD7 and guide RNA specific to X-3 (pMIBa647) was also used in the transformation. The one synthetic middle component was combined with the Blend 2 master mix and transformed into the into S. cerevisiae strain S709-A06 following a yeast electroporation protocol. Transformants were selected on YPD+cloNAT to select for transformants that contain the Mad7 plasmid pMIBa647. Transformants were either picked manually by hand onto YPD plates or by using a Q-pix Colony Picking System (Molecular Devices) to inoculate 1 well of 96-well plate containing YPD media. The plates were grown for 2 days then glycerol was added to 20% final concentration and the plates were stored at −80° C. until needed. Integration of the CBH2 and EG2 construct was verified by PCR with locus specific primers and subsequent sequencing. The strains generated in S709-A06 in this example were designated S1130-B11 and S1130-H11.

### Example 10: Evaluation of Yeast Strains Expressing Polypeptides Having Cellobiohydrolase Activity, Endoglucanase Activity, or Beta-Glucosidase Activity

### Corn Mash Fermentation Procedure

Yeast strains described supra were incubated overnight in 50 mL YPD media (6% w/v D-glucose, 2% peptone, 1% yeast extract) in 125 ml baffled shake flasks at 32° C. at 150 rpm at 32° C. Cells were harvested after 24 hours incubation. Cells were collected by centrifugation and washed in DI water prior to resuspending in 10 ml DI water for dosing. Industrially obtained liquefied corn mash, where liquefaction was carried out using the Fortiva product from Novozymes, was supplemented with 3 ppm penicillin and 500 ppm of urea. Simultaneous saccharification and fermentation (SSF) was performed via mini-scale fermentations. Approximately 4 g of corn mash was added to 15 ml conical tubes. Each tube was dosed with 1×10⁷ cells/g of mash with one of the yeast strains shown in Table X followed by the addition of 0.42 AGU/g of dry solids of an exogenous glucoamylase enzyme product (Innova Ultra F). Eight replicate tube fermentations were conducted for each yeast strain. Glucoamylase and yeast dosages were administered based on the exact weight of corn slurry in each vial. Tubes were incubated at 32° C. and mixed two to three times per day via brief vortex. After 70 hours fermentation time, tubes were centrifuged @3500 rpm for 5 min. Supernatant samples were filtered with 0.2 μm syringe filters into vials for analysis of final ethanol level via HPLC.

### Results

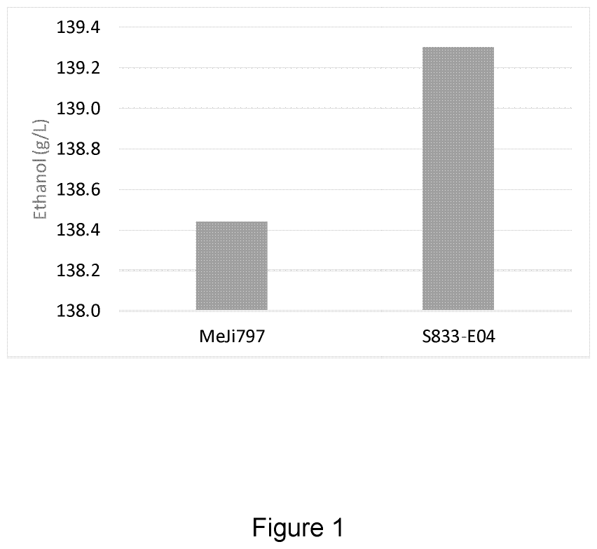

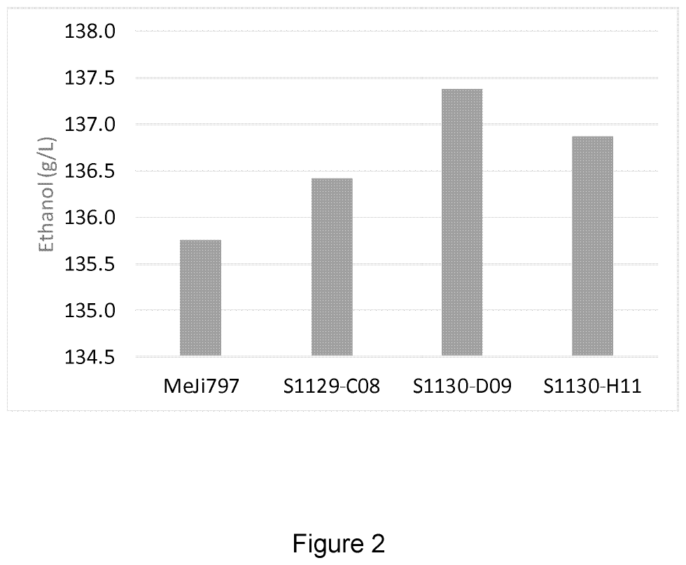

Final ethanol level results from the corn mash fermentation described above are shown in FIGS. 1 and 2. Yeast strain (S833-E04) expressing T. pinophilus beta-glucosidase of SEQ ID NO: 6 showed significantly higher ethanol yield as compare to control strain MeJi797. Yeast strain (S1129-C08) expressing the P. emersonii cellobiohydrolase of SEQ ID NO: 1+Trichoderma reesei endoglucanase of SEQ ID NO: 29+A. fumigatus beta-glucosidase of SEQ ID NO: 8 had higher ethanol yield as compare to control strain MeJi797. Yeast strain (S1130-D09) expressing the A. fumigatus cellobiohydrolase of SEQ ID NO: 9+A. fumigatus beta-glucosidase of SEQ ID NO: 8+Cladosporium antareticum endoglucanase of SEQ ID NO: 5 had higher ethanol yield as compare to control strain MeJi797. Yeast strain (S1130-H11) expressing the Talaromyces verruculosus cellobiohydrolase of SEQ ID NO: 4+A. fumigatus beta-glucosidase of SEQ ID NO: 8+Cladosporium antareticum endoglucanase of SEQ ID NO: 5 had higher ethanol yield as compare to control strain MeJi797.

### Example 11: Evaluation of Ethanol Yield Comparing the Addition an A. nidulans Cellobiohydrolase or P. emersonii Cellobiohydrolase to an A. fumigatus Cellobiohydrolase in Simultaneous Saccharification and Fermentation (SSF)

Liquified corn mash was obtained from Show Me Ethanol plant. A 500 ppm urea and 3 ppm penicillin were added, and slurry pH was adjusted to 5.0 (if needed). The % dry solid (DS) of the mash was determined by HX204 moisture analyzer (Mettler Toledo) at 120 C drying temperature. Prior to SSF, the corn mash was weighed at 40 g in each 125 ml bottle.

A day prior to SSF, yeast propagation was carried out by propagating 50 μl MeJi797 cream yeast in 50 ml 6% YPT medium at 32 C for about 22 hours. The propagated yeast was centrifuged at 3300 rpm for 5 minutes. The precipitate yeast solid was dissolved with 40 ml deionized water and centrifuging 2^{nd} time, then the precipitate solid was suspended with 7.5 ml deionized water. The yeast counting of the suspended yeast solution was carried out with NucleoCounter YC-100 (Chemometec) after 1000× dilution with lysis buffer.

SSF was carried out in three replicates. Enzymes in Table 5 were added accordingly, and the propagated yeast MeJi797 were dosed at 10 million per gram fermentation slurry. The total dry solid of SSF slurry was adjusted to 31% DS with addition of tap water. SSF bottles were placed in an air shaker at 100 rpm and 32 C for 65 hours.

TABLE 5

| | | Activity/ | | |
|---|---|---|---|---|
| | | Protein | | |
| Name | Origin | Content | unit/ml | ID |
| Innova Ultra F | Novozymes | 1450 | AGU/g | NS50527 |
| | glucoamylase product | | | |
| WA Cellulase Complex | Novozymes product | 285.5 | mgEP/ml | WAM00001 |
| cellobiohydrolase | Aspergillus fumigatus | 86.36 | mgEP/ml | P168Y8 |
| | (SEQ ID NO: 9) | | | |
| cellobiohydrolase | Aspergillus nidulans | 3.3 | mgEP/ml | P16C21 |
| | (SEQ ID NO: 2) | | | |
| cellobiohydrolase | Penicillium emersonii | 36 | mgEP/ml | P244YG |
| | (SEQ ID NO: 1) | | | |
| Endoglucanase-1 | Trichoderma reesei | 10.68 | mgEP/ml | P33516 |
| | (SEQ ID NO: 29) | | | |
| beta-glucosidase | Aspergillus fumigatus | 26.5 | mgEP/ml | P3EW |
| | (SEQ ID NO: 8) | | | |
| CBH1&2 double | Trichoderma reesei | 115.9 | mgEP/ml | |
| deletion Tr strain | | | | |

After SSF, fermentation bottles were cooled down in 20 C water for 15 min. Approximately 5 g fermentation slurry was transferred to each 15 ml tube and fermentation was stopped by adding 50 μl 37% H2SO4 and mixing well. Tubes were centrifuged at 3000 rpm for 10 minutes. The supernatant was filtered through 0.2 μm syringe filter in a HPLC vial. Ethanol contents were analyzed with Agilent HPLC system.

The ethanol concentrations from SSF are in Table 6 below. Compared to WA cellulase complex or Af CBH1 mix (a mimic WA complex), it's clear that replacing Aspergillus fumigatus cellobiohydrolase of SEQ ID NO: 9 with the Aspergillus nidulans cellobiohydrolase of SEQ ID NO: 2, or the Penicillium emersonii cellobiohydrolase of SEQ ID NO: 1 resulted in higher ethanol yield.

TABLE 6

| | | Ultra F | | | | | DD Tr | Average | |
|---|---|---|---|---|---|---|---|---|---|
| | Corn | (agu/g | WA | | | Af BG | Strain | Ethanol | Ethanol |
| No: | mash g | DS) | (ug/gDS) | CBHI (ug/g DS) | | ug/g DS | ug/gDS | g/l | STDEV |
| 1 | 40 | 0.42 | | | | | | 135.9 | 0.98 |
| 2 | 40 | 0.42 | 100 | | | | | 136.7 | 0.69 |
| 3 | 40 | 0.42 | | Af CBH1 | 30 | 20 | 50 | 136.5 | 0.31 |
| 4 | 40 | 0.42 | | An CBH1 | 30 | 20 | 50 | 137.1 | 0.45 |
| 5 | 40 | 0.42 | | Pe CBH1 | 30 | 20 | 50 | 137.2 | 0.59 |

### Example 12: Evaluation of Ethanol Yield Impact During SSF for the Addition of a Cladosporium antareticum Endoglucanase

A 500 ppm urea and 3 ppm penicillin was added to liquified corn mash, and the slurry was pH adjusted to 5.0. The % dry solid (DS) of the mash was determined by HX204 moisture analyzer (Mettler Toledo) at 120 C drying temperature. Prior to SSF, the corn mash was pipetted at 3.75 ml (4 g) in each 12 ml tube.

A day prior to SSF, yeast propagation was carried out by propagating 50 μl MeJi797 cream yeast in 50 ml 6% YPT medium at 32 C for about 22 hours. The propagated yeast was centrifuged at 3300 rpm for 5 minutes. The precipitate yeast solid was dissolved with 40 ml deionized water and centrifuged a 2^{nd} time, then the precipitate solid was suspended with 7.5 ml deionized water. The yeast counting of the suspended yeast solution was carried out with NucleoCounter YC-100 (Chemometec) after 1000× dilution with lysis buffer.

SSF was carried out in six replicates. Enzymes in Table 7 were added accordingly to Table 8, and the propagated Innova Force yeast were dosed at 10 million per gram fermentation slurry. And the total dry solid of SSF slurry was adjusted to 33.5% DS with addition of tap water. SSF tubes were placed in 32 C constant temperature chamber for 66 hours, tubes were vortexed once a day.

TABLE 7

| No | Name | Origin | Activity (unit) | | P# |
|---|---|---|---|---|---|
| 1 | Innova Ultra F | Novozymes | 1450 | AGU/g | |
| | | glucoamylase product | | | |
| 2 | WA cellulase | Novozymes product | 285.5 | mgEP/ml | WAM00001 |
| | complex | | | | |
| 3 | Endoglucanase-1 | Trichoderma reesei | 10.68 | mgEP/ml | P33516 |
| | | (SEQ ID NO: 29) | | | |
| 4 | Endoglucanase-2 | Trichoderma reesei | 9.21 | mgEP/ml | P24KEK |
| | | (SEQ ID NO: 57) | | | |
| 5 | Endoglucanase-2 | Cladosporium antareticum | 7.3 | mgEP/ml | P64DAT |
| | | (SEQ ID NO: 5) | | | |
| 6 | cellobiohydrolase | Aspergillus fumigatus | 86.36 | mgEP/ml | P3EX |
| | | (SEQ ID NO: 9) | | | |
| 7 | cellobiohydrolase | Penicillium emersonii | 36 | mgEP/ml | P244YG |
| | | (SEQ ID NO: 1) | | | |
| 8 | cellobiohydrolase | Aspergillus nidulans | 3.3 | mgEP/ml | P16C21 |
| | | (SEQ ID NO: 2) | | | |
| 9 | beta-glucosidase | Aspergillus fumigatus | 26.5 | mgEP/ml | P23VYK |
| | | (SEQ ID NO: 58) | | | |
| 9 | beta-glucosidase | Talaromyces pinophilus | 2.7 | mgEP/ml | P23WFT |
| | | (SEQ ID NO: 6) | | | |
| 10 | QD Tr Strain | Trichoderma reesei | 34.7 | mgEP/ml | |

Note:
QD Tr Strain is Trichoderma reesei strain with gene deletion of EG1, GH11 xylanase, CBH1 and CBH2.

TABLE 8

| | | | | Ca | Af | Pe | An | Af | QD Tr |
|---|---|---|---|---|---|---|---|---|---|
| | Ultra F | Tr EG1 | Tr EG2 | EG2 | CBHI | CBH1 | CBH1 | BG4M | Strain |
| | AGU/g | ug/gDS | ug/gDS | ug/gDS | ug/gDS | ug/gDS | ug/gDS | ug/gDS | ug/gDS |
| 1 | 0.42 | | | | | | | | |
| 2 | 0.42 | 5 | | | 15 | | | 10 | 15 |
| 3 | 0.42 | 5 | 5 | | 15 | | | 10 | 15 |
| 4 | 0.42 | 5 | | 5 | 15 | | | 10 | 15 |
| 5 | 0.42 | 5 | | | | 15 | | 10 | 15 |
| 6 | 0.42 | 5 | 5 | | | 15 | | 10 | 15 |
| 7 | 0.42 | 5 | | 5 | | 15 | | 10 | 15 |
| 8 | 0.42 | 5 | | | | | 15 | 10 | 15 |
| 9 | 0.42 | 5 | 5 | | | | 15 | 10 | 15 |
| 10 | 0.42 | 5 | | 5 | | | 15 | 10 | 15 |

After SSF, fermentation tubes were cooled down in 20 C water for 15 min. The fermentation was stopped by adding 50 μl 38% H2SO4 in each tube and mixing well. Tubes were centrifuged at 3000 rpm for 10 minutes. The supernatant was filtered through 0.2 μm syringe filter in a HPLC vial. Ethanol contents were analyzed with Agilent HPLC system.

The ethanol concentrations from SSF are in Table 9 below. The results show that the addition of any CBH1 cellulase mix resulted in higher ethanol yield compared to no cellulase control. The addition of either Trichoderma reesei endoglucanase-2 of SEQ ID NO: 57, or Cladosporium antareticum endoglucanase-2 of SEQ ID NO: 5 increased the ethanol yield compared to without the endoglucanase-2. The addition of Cladosporium antareticum endoglucanase-2 of SEQ ID NO: 5 in each cellobiohydrolase cellulase mixture resulted the highest ethanol yield.

TABLE 9

| | Average | | | | STDEV | | | |
|---|---|---|---|---|---|---|---|---|
| | no | w/o | w/ Tr | w/ Ca | no | w/o | w/ Tr | w/ Ca |
| | Cellulase | EG2 | EG2 | EG2 | Cellulase | EG2 | EG2 | EG2 |
| UF w/o cellulase | 138.84 | | | | 0.69 | | | |
| UF + Af CBH1 Mix | | 139.04 | 139.43 | 139.76 | | 0.49 | 0.55 | 0.65 |
| UF + Pe CBH1 Mix | | 139.25 | 139.45 | 139.68 | | 0.37 | 0.53 | 0.66 |
| UF + An CBH1 Mix | | 139.25 | 139.35 | 140.02 | | 0.77 | 0.64 | 0.20 |

### Example 13: Evaluation of Ethanol Yield Impact During SSF with the Addition of a Talaromyces pinophilus Beta-Glucosidase

A 500 ppm urea and 3 ppm penicillin was added to liquified corn mash, and the slurry was pH adjusted to 5.0. The % dry solid (DS) of the mash was determined by HX204 moisture analyzer (Mettler Toledo) at 120 C drying temperature. Prior to SSF, the corn mash was weighed at 40 g in each 125 ml bottle.

A day prior to SSF, yeast propagation was carried out by propagating 50 μl MeJi797 cream yeast in 50 ml 6% YPT medium at 32 C for about 22 hours. The propagated yeast was centrifuged at 3300 rpm for 5 minutes. The precipitate yeast solid was dissolved with 40 ml deionized water and centrifuging 2^{nd} time, then the precipitate solid was suspended with 7.5 ml deionized water. The yeast counting of the suspended yeast solution was carried out with NucleoCounter YC-100 (Chemometec) after 1000× dilution with lysis buffer.

SSF was carried out in three replicates. Enzymes of Table 10 below were added accordingly, and the propagated Innova Force yeast were dosed at 10 million per gram fermentation slurry. And the total dry solid of SSF slurry was adjusted to 34.5% DS with addition of tap water. SSF bottles were placed in an air shaker at 100 rpm and 32 C for 66 hours.

TABLE 10

| No | Name | Origin | Activity (unit) | | ID |
|---|---|---|---|---|---|
| 1 | Innova Ultra F | Novozymes | 1450 | AGU/g | NS50527 |
| | | glucoamylase product | | | |
| 2 | cellobiohydrolase | Aspergillus fumigatus | 86.36 | mgEP/ml | P3EX |
| | | (SEQ ID NO: 9) | | | |
| 3 | cellobiohydrolase | Penicillium emersonii | 36 | mgEP/ml | P244YG |
| | | (SEQ ID NO: 1) | | | |
| 4 | Endoglucanase-1 | Trichoderma reesei | 10.68 | mgEP/ml | P33516 |
| | | (SEQ ID NO: 29) | | | |
| 5 | Endoglucanase-2 | Cladosporium antareticum | 7.3 | mgEP/ml | P64DAT |
| | | (SEQ ID NO: 5) | | | |
| 6 | QD Tr Strain | Trichoderma reesei | 34.7 | mgEP/ml | |
| 7 | beta-glucosidase | Aspergillus fumigatus | 26.5 | mgEP/ml | P23VYK |
| | | (SEQ ID NO: 58) | | | |
| 8 | beta-glucosidase | Talaromyces pinophilus | 2.7 | mgEP/ml | P23WFT |
| | | (SEQ ID NO: 6) | | | |

Note:
QD Tr Strain is Trichoderma reesei strain with gene deletion of EG1, GH11 xylanase, CBH1 and CBH2.

After SSF, fermentation bottles were cooled down in 20 C water for 15 min. Approximately 5 g fermentation slurry was transferred to each 15 ml tube and fermentation was stopped by adding 50 μl 38% H2SO4 and mixing well. Tubes were centrifuged at 3000 rpm for 10 minutes. The supernatant was filtered through 0.2 μm syringe filter in a HPLC vial. Ethanol contents were analyzed with Agilent HPLC system.

The ethanol concentrations from SSF are in Table 11 below. By replacing Aspergillus fumigatus beta-glucosidase of SEQ ID NO: 58 with the Talaromyces pinophilus beta-glucosidase in two different cellobiohydrolase cellulase mixtures, a higher ethanol yield was observed.

TABLE 11

| Corn | | | | | | | | Ca | Average | |
|---|---|---|---|---|---|---|---|---|---|---|
| mash | Ultra F | | | Af BG4M | Tb BG | QD Tr | Tr EG1 | EG2 | Ethanol | STDE [symbol] |
| g | agu/gDS | CBHI (ug/gDS) | | ug/gDS | ug/gDS | ug/gDS | ug/gDS | ug/gDS | g/l | Ethano [symbol] |
| 40 | 0.42 | AfCBH1 | 30 | 20 | | 30 | 10 | 10 | 141.45 | 0.54 |
| 40 | 0.42 | PeCBH1 | 30 | 20 | | 30 | 10 | 10 | 141.62 | 0.28 |
| 40 | 0.42 | AfCBH1 | 30 | | 20 | 30 | 10 | 10 | 141.57 | 0.44 |
| 40 | 0.42 | PeCBH1 | 30 | | 20 | 30 | 10 | 10 | 141.88 | 0.38 |

[symbol]

indicates data missing or illegible when filed

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### Example 14

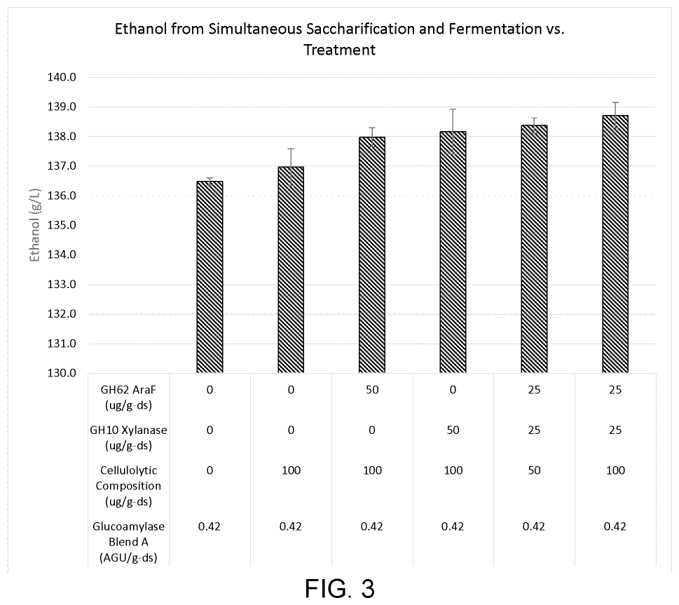

This example illustrates the synergy between an enzyme blend comprising a GH10 xylanase and GH62 arabinofuranosidase enhances the ethanol yield during simultaneous saccharification and fermentation in conjunction with a cellulolytic composition derived from Trichoderma reesei and a glucoamylase blend. Simultaneous saccharification and fermentation (SSF) was performed using industrial corn mash liquefied using Alpha-amylase blend X (Novozymes A/S). An industrial yeast strain (Ethanol Red; Lesaffre) was cultivated for 18 hours in standard YPD media containing 6% glucose at 32° C. and 120 RPM. After the cultivation the yeast propagation was centrifuged, the supernatant decanted, and the yeast cells resuspended in deionized water. The resuspended cells were enumerated using a NucleoCounter YC-100 (ChemoMetric, Allerod, Denmark). The corn mash was supplemented with 500 ppm of urea and dosed with 0.42 AGU/g-DS of an exogenous glucoamylase blend (Glucoamylase Blend A; Novozymes A/S) and/or 50-100 μg/g-DS of an exogenous Cellulolytic Composition (Novozymes A/S), and/or 25-50 μg/g-DS of an exogenous xylanase product containing a GH10 xylanase (Novozymes A/S), and/or 25-50 μg/g-DS of an exogenous xylanase product containing a GH62 arabinofuranosidase (Novozymes A/S). Approximately 50 g of corn mash with a solids content of 34.5% wt. dry solids was dispensed into a 250 ml glass media bottle (Thermo Fisher Scientific, Hampton, NH) for each sample, followed by the addition of approximately 10\hat{ }8 yeast cells/g of corn mash from the overnight culture. Bottles were incubated at 32° C. on an orbital shaker at 150 RPM for 52 hr. Fermentation was stopped by the addition of 500 μL of 42% sulfuric acid followed by centrifugation at 3000 RPM for 10 minutes. The supernatant was analyzed for ethanol using HPLC-RID. The fermentation results with the combination of Cellulolytic Composition, GH10 xylanase, and GH62 arabinofuranosidase is shown in FIG. 3.

### Example 15

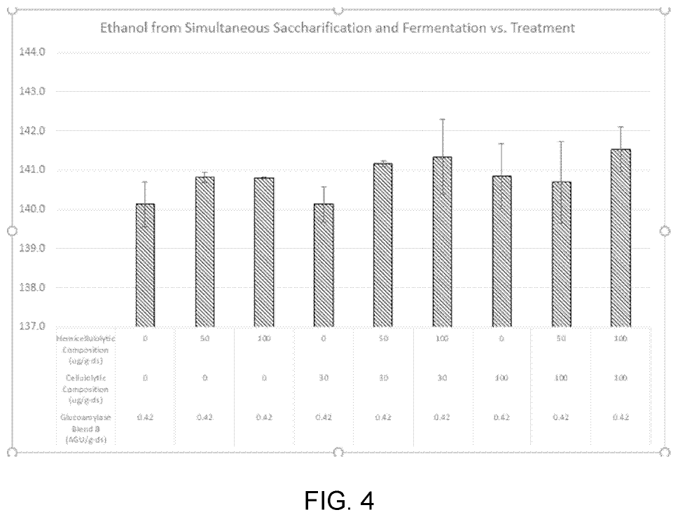

This example illustrates the synergy between an enzyme blend comprising a GH10 xylanase and GH62 arabinofuranosidase enhances the ethanol yield during simultaneous saccharification and fermentation in conjunction with a cellulolytic composition derived from Trichoderma reesei and a glucoamylase blend. Simultaneous saccharification and fermentation (SSF) was performed using industrial corn mash liquified using Alpha-amylase blend X (Novozymes A/S). An industrial yeast Saccharomyces strain MEJI797 (Novozymes A/S) was cultivated for 18 hours in standard YPD media containing 6% glucose at 32° C. and 120 RPM. After the cultivation the yeast propagation was centrifuged, the supernatant decanted, and the yeast cells resuspended in deionized water. The resuspended cells were enumerated using a NucleoCounter YC-100 (ChemoMetric, Allerod, Denmark). The corn mash was supplemented with 500 ppm of urea and dosed with 0.42 AGU/g-DS of an exogenous glucoamylase blend (Glucoamylase Blend B; Novozymes A/S) and/or 50-100 μg/g-DS of an exogenous Cellulolytic Composition (Novozymes A/S), and/or 50-100 μg/g-DS of an exogenous xylanase product containing a GH10 xylanase and GH62 arabinofuranosidase (Hemicellulolytic Composition; Novozymes A/S). Approximately 50 g of corn mash with a solids content of 33.0% wt. dry solids was dispensed into a 250 ml glass media bottle (Thermo Fisher Scientific, Hampton, NH) for each sample, followed by the addition of approximately 10\hat{ }8 yeast cells/g of corn mash from the overnight culture. Bottles were incubated at 32° C. on an orbital shaker at 150 RPM for 68 hr. Fermentation was stopped by the addition of 500 μL of 42% sulfuric acid followed by centrifugation at 3000 RPM for 10 minutes. The supernatant was analyzed for ethanol using HPLC-RID. The fermentation results with the combination of Cellulolytic Composition and Hemicellulolytic Composition is shown in FIG. 4.

1. A process for producing a fermentation product from starch-containing material, the process comprising the steps of:
i) liquefying the starch-containing material at a temperature above the initial gelatinization temperature using an alpha-amylase;
ii) saccharifying the liquefied starch-containing;
iii) fermenting saccharified starch-containing material using a fermenting organism;
wherein at least one polypeptide having cellobiohydrolase activity, endoglucanase activity, or beta-glucosidase activity is present or added during fermentation; and
wherein the at least one polypeptide having cellobiohydrolase activity, endoglucanase activity, or beta-glucosidase activity comprises at least one polypeptide selected from the group consisting of:
(i) the mature polypeptide of SEQ ID NO: 3, or a polypeptide having at least 70% identity to SEQ ID NO: 3;
(ii) the mature polypeptide of SEQ ID NO: 5, or a polypeptide having at least 70% identity to SEQ ID NO: 5; and
(iii) the mature polypeptide of SEQ ID NO: 1, or a polypeptide having at least 70% identity to SEQ ID NO: 1;
(iv) the mature polypeptide of SEQ ID NO: 2, or a polypeptide having at least 70% identity to SEQ ID NO: 2;
(v) the mature polypeptide of SEQ ID NO: 4, or a polypeptide having at least 70% identity to SEQ ID NO: 4; and
(vi) the mature polypeptide of SEQ ID NO: 6, or a polypeptide having at least 70% identity to SEQ ID NO: 6.

2. The process of claim 1, wherein the at least one polypeptide having cellobiohydrolase activity, endoglucanase activity, or beta-glucosidase activity comprises or consists of SEQ ID NO: 3, SEQ ID NO: 5, SEQ ID NO: 1, SEQ ID NO: 2, SEQ ID NO: 4, or SEQ ID NO: 6, or the mature polypeptide of SEQ ID NO: 3, SEQ ID NO: 5, SEQ ID NO: 1, SEQ ID NO: 2, SEQ ID NO: 4, or SEQ ID NO: 6.

3. The process of claim 1, wherein the mature polypeptide is amino acids 26 to 533 of SEQ ID NO: 3, amino acids 20 to 410 of SEQ ID NO: 5, amino acids 19 to 520 of SEQ ID NO: 1, amino acids 1 to 503 of SEQ ID NO: 2, amino acids 20 to 456 of SEQ ID NO: 4, and amino acids 20 to 855 of SEQ ID NO: 6.

4. The process of claim 1, wherein the at least one polypeptide having cellobiohydrolase activity, endoglucanase activity, or beta-glucosidase activity is a variant of the mature polypeptide of SEQ ID NO: 3, SEQ ID NO: 5, SEQ ID NO: 1, SEQ ID NO: 2, SEQ ID NO: 4, SEQ ID NO: 6 comprising a substitution, deletion, and/or insertion at one or more positions.

5. The process of claim 1, wherein the at least one polypeptide having cellobiohydrolase activity, endoglucanase activity, or beta-glucosidase activity is a fragment of SEQ ID NO: 3, SEQ ID NO: 5, SEQ ID NO: 1, SEQ ID NO: 2, SEQ ID NO: 4, or SEQ ID NO: 6, wherein the fragment has cellobiohydrolase activity, endoglucanase activity, or beta-glucosidase activity.

6. An isolated polypeptide having cellobiohydrolase activity, endoglucanase activity, or beta-glucosidase activity selected from the group consisting of:
(i) the mature polypeptide of SEQ ID NO: 3, or a polypeptide having at least 70% identity to SEQ ID NO: 3;
(ii) the mature polypeptide of SEQ ID NO: 5, or a polypeptide having at least 70% identity to SEQ ID NO: 5;
(iii) the mature polypeptide of SEQ ID NO: 1, or a polypeptide having at least 70% identity to SEQ ID NO: 1;
(iv) the mature polypeptide of SEQ ID NO: 2, or a polypeptide having at least 70% identity to SEQ ID NO: 2;
(v) the mature polypeptide of SEQ ID NO: 4, or a polypeptide having at least 70% identity to SEQ ID NO: 4; and
(vi) the mature polypeptide of SEQ ID NO: 6, or a polypeptide having at least 70% identity to SEQ ID NO: 6.

7. The polypeptide of claim 6, wherein the amino acid sequence comprises or consists of SEQ ID NO: 3, SEQ ID NO: 5, SEQ ID NO: 1, SEQ ID NO: 2, SEQ ID NO: 4, or SEQ ID NO: 6, or the mature polypeptide of SEQ ID NO: 3, SEQ ID NO: 5, SEQ ID NO: 1, SEQ ID NO: 2, SEQ ID NO: 4, or SEQ ID NO: 6.

8. The polypeptide of claim 6, wherein the mature polypeptide is amino acids 26 to 533 of SEQ ID NO: 3, amino acids 20 to 410 of SEQ ID NO: 5, amino acids 19 to 520 of SEQ ID NO: 1, amino acids 1 to 503 of SEQ ID NO: 2, amino acids 20 to 456 of SEQ ID NO: 4, and amino acids 20 to 855 of SEQ ID NO: 6.

9. The polypeptide of claim 6, which is a variant of the mature polypeptide of SEQ ID NO: 3, SEQ ID NO: 5, SEQ ID NO: 1, SEQ ID NO: 2, SEQ ID NO: 4, or SEQ ID NO: 6 comprising a substitution, deletion, and/or insertion at one or more positions.

10. The polypeptide of claim 6, which is a fragment of SEQ ID NO: 3, SEQ ID NO: 5, SEQ ID NO: 1, SEQ ID NO: 2, SEQ ID NO: 4, or SEQ ID NO: 6, wherein the fragment has cellobiohydrolase activity, endoglucanase activity, or beta-glucosidase activity.

11. An isolated polynucleotide encoding the polypeptide of claim 6.

12. A nucleic acid construct or expression vector comprising the polynucleotide of claim 11 operably linked to one or more control sequences that direct the production of the polypeptide in an expression host.

11. A recombinant host cell comprising the polynucleotide of claim 11 operably linked to one or more control sequences that direct the production of the polypeptide.

12. A method of producing a polypeptide having cellobiohydrolase activity, endoglucanase activity, or beta-glucosidase activity, comprising:
(a) cultivating the host cell of claim 11 under conditions conducive for production of the polypeptide; and
(b) recovering the polypeptide.

13. (canceled)

14. A recombinant host cell comprising at least one heterologous polynucleotide encoding a polypeptide having cellobiohydrolase activity, endoglucanase activity, or beta-glucosidase activity selected from the group consisting of:
(i) the mature polypeptide of SEQ ID NO: 3, or a polypeptide having at least 70% identity to SEQ ID NO: 3;
(ii) the mature polypeptide of SEQ ID NO: 5, or a polypeptide having at least 70% identity to SEQ ID NO: 5;
(iii) the mature polypeptide of SEQ ID NO: 1, or a polypeptide having at least 70% identity to SEQ ID NO: 1;
(iv) the mature polypeptide of SEQ ID NO: 2, or a polypeptide having at least 70% identity to SEQ ID NO: 2;
(v) the mature polypeptide of SEQ ID NO: 4, or a polypeptide having at least 70% identity to SEQ ID NO: 4; and
(vi) the mature polypeptide of SEQ ID NO: 6, or a polypeptide having at least 70% identity to SEQ ID NO: 6.

15. The recombinant host cell of claim 14, wherein the at least one heterologous polynucleotide encoding a polypeptide having cellobiohydrolase activity, endoglucanase activity, or beta-glucosidase activity comprises or consists of SEQ ID NO: 3, SEQ ID NO: 5, SEQ ID NO: 1, SEQ ID NO: 2, SEQ ID NO: 4, or SEQ ID NO: 6, or the mature polypeptide of SEQ ID NO: 3, SEQ ID NO: 5, SEQ ID NO: 1, SEQ ID NO: 2, SEQ ID NO: 4, or SEQ ID NO: 6.

16. The recombinant host cell of claim 14, wherein the mature polypeptide is amino acids 26 to 533 of SEQ ID NO: 3, amino acids 20 to 410 of SEQ ID NO: 5, amino acids 19 to 520 of SEQ ID NO: 1, amino acids 1 to 503 of SEQ ID NO: 2, amino acids 20 to 456 of SEQ ID NO: 4, and amino acids 20 to 855 of SEQ ID NO: 6.

17. The recombinant host cell of claim 14, wherein the at least one heterologous polynucleotide encoding a polypeptide having cellobiohydrolase activity, endoglucanase activity, or beta-glucosidase activity is a variant of the mature polypeptide of SEQ ID NO: 3, SEQ ID NO: 5, SEQ ID NO: 1, SEQ ID NO: 2, SEQ ID NO: 4, or SEQ ID NO: 6 comprising a substitution, deletion, and/or insertion at one or more positions.

18. The recombinant host cell of claim 14, wherein the at least one heterologous polynucleotide encoding a polypeptide having cellobiohydrolase activity, endoglucanase activity, or beta-glucosidase activity is a fragment of SEQ ID NO: 3, SEQ ID NO: 5, SEQ ID NO: 1, SEQ ID NO: 2, SEQ ID NO: 4, or SEQ ID NO: 6, wherein the fragment has cellobiohydrolase activity, endoglucanase activity, or beta-glucosidase activity.

19. The recombinant host cell of claim 14, wherein the cell further comprises a heterologous polynucleotide encoding a glucoamylase.

20. The recombinant host cell of claim 19, wherein the heterologous polynucleotide encoding the glucoamylase is operably linked to a promoter that is foreign to the polynucleotide.

21. The recombinant host cell of claim 14, wherein the cell further comprises a heterologous polynucleotide encoding an alpha-amylase.

22. The recombinant host cell of claim 21, wherein the heterologous polynucleotide encoding the alpha-amylase is operably linked to a promoter that is foreign to the polynucleotide.

23. The recombinant host cell of claim 14, wherein the cell is a yeast cell.

24. The recombinant host cell of claim 14, wherein the cell is a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell.

25. The recombinant host cell of claim 14, wherein the cell is a Saccharomyces cerevisiae cell.
